SEQUENCE LISTING (31 sequences)

SEQ ID NO: 1 - DNA, 21 bp, Artificial
  PCR primer
cgcagatcacccagaagatcg

SEQ ID NO: 2 - DNA, 20 bp, Artificial
  PCR primer
ttcgtcgcatttgtccacca

SEQ ID NO: 3 - DNA, 20 bp, Artificial
  PCR primer
cagaggcagcggaatcgtaa

SEQ ID NO: 4 - DNA, 21 bp, Artificial
  PCR primer
tgaagctctagctcggtgttc

SEQ ID NO: 5 - DNA, 20 bp, Artificial
  PCR primer
atatggttcctggcaagccc

SEQ ID NO: 6 - DNA, 20 bp, Artificial
  PCR primer
gtggggtggcaggtattagg

SEQ ID NO: 7 - DNA, 20 bp, Artificial
  PCR primer
tggtacagaactgcagcgag

SEQ ID NO: 8 - DNA, 20 bp, Artificial
  PCR primer
agccagaactggtcttcgtg

SEQ ID NO: 9 - DNA, 20 bp, Artificial
  PCR primer
gtggcagagggagaatgtcg

SEQ ID NO: 10 - DNA, 23 bp, Artificial
  PCR primer
taaaacagcataaccttttgcga

SEQ ID NO: 11 - DNA, 17 bp, Artificial
  PCR primer
gagccaacgggtcatct

SEQ ID NO: 12 - DNA, 20 bp, Artificial
  PCR primer
gaggggccatccacagtctt

SEQ ID NO: 13 - DNA, 21 bp, Artificial
  PCR primer
tgtccaaggtggtaaagggtg

SEQ ID NO: 14 - DNA, 21 bp, Artificial
  PCR primer
ccggcgatttaactgatcctg

SEQ ID NO: 15 - DNA, 21 bp, Artificial
  PCR primer
gctggacgtgaccatcatgta

SEQ ID NO: 16 - DNA, 19 bp, Artificial
  PCR primer
gggccgtataggaacgtgc

SEQ ID NO: 17 - DNA, 23 bp, Artificial
  PCR primer
ggtgcgagagcgtcagtattaag

SEQ ID NO: 18 - DNA, 19 bp, Artificial
  PCR primer
agctccctgcttgcccata

SEQ ID NO: 19 - DNA, 29 bp, Artificial
  PCR probe
aaaattcggttaaggccagggggaaagaa

SEQ ID NO: 20 - DNA, 21 bp, Artificial
  PCR primer
gggcttcctcctccaagtctt

SEQ ID NO: 21 - DNA, 18 bp, Artificial
  PCR primer
ccgcttcgagacctttgc

SEQ ID NO: 22 - DNA, 20 bp, Artificial
  PCR primer
ggtggtccccagtaatgtgg

SEQ ID NO: 23 - DNA, 21 bp, Artificial
  PCR primer
cgtcaagattccgatggtcct

SEQ ID NO: 24 - DNA, 21 bp, Artificial
  PCR primer
tgtccaaggtggtaaagggtg

SEQ ID NO: 25 - DNA, 21 bp, Artificial
  PCR primer
ccggcgatttaactgatcctg

SEQ ID NO: 26 - DNA, 20 bp, Artificial
  PCR primer
atgtcgtctggtccctgttc

SEQ ID NO: 27 - DNA, 20 bp, Artificial
  PCR primer
gtcatgaggatgcccagaat

SEQ ID NO: 28 - DNA, 21 bp, Artificial
  PCR primer
ctgtcctgtgtgtcagaccag

SEQ ID NO: 29 - DNA, 21 bp, Artificial
  PCR primer
tgtgggctcatcttgacctct

SEQ ID NO: 30 - DNA, 21 bp, Artificial
  PCR primer
gctggacgtgaccatcatgta

SEQ ID NO: 31 - DNA, 19 bp, Artificial
  PCR primer
gggccgtataggaacgtgc

(19) United States
(12) Patent Application Publication
(10) Pub. No.: US 2019/0284286 A1
(43) Pub. Date: Sep. 19, 2019

(54) Modulation of Type I Interferons to Reactivate HIV-1 Reservoir and Enhance HIV-1 Treatment

(71) Applicant: The University of North Carolina at Chapel Hill, Chapel Hill, NC (US)

(72) Inventors: Liang Cheng, Chapel Hill, NC (US); Lishan Su, Chapel Hill, NC (US)

(21) Appl. No.: 16/340,968

(22) PCT Filed: Oct. 25, 2017
(86) PCT No.: PCT/US2017/058183

§ 371 Date: Apr. 10, 2019

Related U.S. Application Data
(60) Provisional application No. 62/413,661, filed on Oct. 27, 2016.

(51) Int. Cl. C07K 16/28; A61K 39/395; A61K 45/06; A61K 49/00; A61P 31/18

(52) U.S. Cl. CPC C07K 16/2866; A61K 39/3955; C07K 16/2851; A61K 2039/505; A61K 49/0008; A61P 31/18; A61K 45/06

(57) ABSTRACT

The present invention relates to methods for reactivating latent HIV-1, treating HIV-1 infection in a subject, and increasing the effectiveness of combination antiretroviral therapy by inhibiting type I interferon signaling. The invention further relates to methods of screening for HIV-1 therapeutics.

### STATEMENT OF PRIORITY

This application claims the benefit of U.S. Provisional Application Ser. No. 62/413,661, filed Oct. 27, 2016, the entire contents of which are incorporated by reference herein.

### STATEMENT OF GOVERNMENT SUPPORT

This invention was made with government support under AI095097 awarded by the National Institutes of Health. The government has certain rights in the invention.

### FIELD OF THE INVENTION

The present invention relates to methods for reactivating latent HIV-1, treating HIV-1 infection in a subject, and increasing the effectiveness of combination antiretroviral therapy by inhibiting type I interferon signaling. The invention further relates to methods of screening for HIV-1 therapeutics.

### BACKGROUND OF THE INVENTION

Type I interferons (IFN-I) are critical for controlling virus infections (Zuniga et al., Curr. Top. Microbiol. Immunol. 316:337 (2007); Schoggins et al., Nature 472:481 (2011)), but they also contribute to impaired host immunity and virus persistence (Wilson et al., Science 340:202 (2013); Teijaro et al., Science 340:207 (2013)). The precise role of IFN-I during chronic human immunodeficiency virus type 1 (HIV-1) infection remains unclear (Doyle et al., Nat. Rev. Microbiol. 13:403 (2015); Bosinger et al., Curr. HIV/AIDS Rep. 12:41 (2015)). HIV-1 infection induces widespread expression of IFN-I and interferon stimulated genes (ISGs) (Stacey et al., J. Virol. 83:3719 (2009); Hardy et al., PLoS One 8:e56527 (2013)). It has been reported that IFN-I can suppress HIV-1 replication in vitro (Doyle et al., Nat. Rev. Microbiol. 13:403 (2015)), and the major anti-HIV-1 restriction factors are encoded by ISGs (Doyle et al., Nat. Rev. Microbiol. 13:403 (2015)). In addition, IFN-I has been shown to inhibit early HIV-1 infection in humanized mice (Lavender et al., J. Virol. 90:6001 (2016)) and simian immunodeficiency virus (SIV) infection in rhesus macaque in vivo (Sandler et al., Nature 511:601 (2014)). These observations suggest that a robust type I IFN response helps to control or limit initial HIV-1 and SIV infection.

IFN-I has also been implicated in the immunopathogenesis of AIDS during chronic HIV-1 infection (Doyle et al., Nat. Rev. Microbiol. 13:403 (2015); Bosinger et al., Curr. HIV/AIDS Rep. 12:41 (2015)). Studies using nonhuman primate models have documented that sustained IFN-I signaling is associated with pathogenic SIV infection (Jacquelin et al., J. Clin. Invest. 119:3544 (2009); Bosinger et al., J Clin. Invest. 119:3556 (2009); Harris et al., J. Virol. 84:7886 (2010); Favre et al., PLoS Pathog. 5:e1000295 (2009)). IFN-I is induced during acute phase of SIV infection in both pathogenic (rhesus macaques or pigtail macaques) and non-pathogenic hosts (African green monkeys or sooty mangabeys). However, compared to the nonpathogenic natural SW infection, pathogenic SIV infection leads to AIDS development, associated with sustained IFN-I signaling (Jacquelin et al., J. Clin. Invest. 119:3544 (2009); Bosinger et al., J. Clin. Invest. 119:3556 (2009); Harris et al., J. Virol. 84:7886 (2010); Favre et al., PLoS Pathog. 5:e1000295 (2009)). Furthermore, studies in HIV-1 infected patients indicate that expression of IFN-I and ISGs is correlated with higher level of viral load, enhanced hyper-immune activation and faster disease progression (Hardy et al., PLoS One 8:e56527 (2013); Rotger et al., PLoS Pathog. 6:e1000781 (2010); Hyrcza et al., J. Virol. 81:3477 (2007); Sedaghat et al., J. Virol. 82:1870 (2008)). Using the mouse model of LCMV persistent infection, it is reported that blocking IFN-I signaling by IFNaR antibody can reverse immune suppression, restore lymphoid architecture and accelerate clearance of the virus (Wilson et al., Science 340:202 (2013); Teijaro et al., Science 340:207 (2013)).

Administration of exogenous IFN-α can lower HIV-1 burden in HIV-1 infected patients but fails to show a significant benefit in HIV-1 disease progression (Bosinger et al., Curr. HIV/AIDS Rep. 2:41 (2015)). Interestingly, recent studies report that the administration of IFN-α in HIV-1 mono-infected or HIV-1\HCV co-infected patients results in reduction of cell associated viral RNA and DNA in the blood (Azzoni et al., J. Infect. Dis. 207:213 (2013); Sun et al., J. Infect. Dis. 209:1315 (2014); Jiao et al., Antiviral Res. 118:118 (2015); Moron-Lopez et al., J. Infect. Dis. 213:1008 (2016)). However, other studies in HIV-1 infected patients indicate that persistent expression of ISGs is correlated with higher viral load, enhanced hyper-immune activation and faster disease progression (Hardy et al., PLoS One 8:e56527 (2013); Rotger et al., PLoS Pathog. 6:e1000781 (2010); Hyrcza et al., J. Virol. 81:3477 (2007); Sedaghat et al., J. Virol. 82:1870 (2008)). In addition, administration of IFN-I to patients also leads to a decrease in CD4 T-cell count (Azzoni et al., J. Infect. Dis. 207:213 (2013); Moron-Lopez et al., J. Infect. Dis. 213:1008 (2016)) and enhanced CD8 T cell activation (Manion et al., PLoS One 7:e30306 (2012)) (22) in the blood. Moreover, despite efficient suppression of HIV-1 replication with combined antiretroviral therapy (cART), abnormally elevated IFN-I signaling persists in some patients even under extensive cART (Fernandez et al., J. Infect. Dis. 204:1927 (2011); Dunham et al., J. Acquir. Immune Defic. Syndr. 65:133 (2014)), which may impede the reversion of hyper-immune activation and immune recovery in those immune non-responder patients (Deeks, Annu. Rev. Med. 62:141 (2011)). These reports highlight that IFN-I may play important but complex roles in HIV-1 persistent infection and pathogenesis.

The present invention addresses previous shortcomings in the art by providing methods for reactivating virus during chronic HIV-1 infection and enhancing treatment of HIV-1.

### SUMMARY OF THE INVENTION

The present invention is based in part on the finding that IFNaR blockade during persistent HIV-1 infection reversed HIV-1-induced immune hyper-activation, rescued anti-HIV-1 immune responses and reduced the size of HIV-1 reservoirs in lymphoid tissues in the presence of cART. This method provides a strategy to enhance immune recovery and to reduce HIV-1 reservoirs in those patients with sustained IFN-I signaling during suppressive cART.

Accordingly, in one aspect, the invention relates to a method of reactivating latent HIV-1 in a subject in need thereof, comprising inhibiting type I interferon signaling in the subject, thereby reactivating latent HIV-1 in the subject.

Another aspect of the invention relates to a method of reducing HIV-1 reservoirs in a subject in need thereof, comprising inhibiting type I interferon signaling in the subject, thereby reducing HIV-1 reservoirs in the subject.

A further aspect of the invention relates to a method of treating HIV-1 infection in a subject in need thereof, comprising inhibiting type I interferon signaling in the subject, thereby treating HIV-1 infection in the subject.

An additional aspect of the invention relates to a method of increasing the effectiveness of combination antiretroviral therapy (cART) for HIV-1 infection in a subject in need thereof, comprising inhibiting type I interferon signaling in the subject prior to, during, and/or after cART, thereby increasing the effectiveness of cART in the subject.

Another aspect of the invention relates to a method of inhibiting immune hyperactivation in a subject in need thereof, comprising inhibiting interferon-I signaling in the subject, thereby inhibiting immune hyperactivation in the subject.

A further aspect of the invention relates to a method for identifying a compound suitable for treatment of HIV-1 infection, the method comprising providing an animal model of HIV-1 infection in which type I interferon signaling has been inhibited, delivering a candidate compound to the animal, and measuring HIV-1 levels in the animal, wherein a decrease in HIV-1 levels compared to an animal that has not received the candidate compound identifies the candidate compound as a compound suitable for treatment of HIV-1 infection.

These and other aspects of the invention are set forth in more detail in the description of the invention below.

### BRIEF DESCRIPTION OF THE DRAWINGS

FIGS. 1A-1F show cART efficiently inhibits HIV-1 replication but fails to reverse inflammation and clear HIV-1 reservoirs in humanized mice. (A-B) Humanized mice infected with HIV-1 were treated with cART) from 4.5 to 11.5 wpi. (A) HIV-1 RNA levels in the plasma of HIV-1 infected (n=3) and HIV-1 infected cART-treated mice (n=7) at indicated time points. (B) Percentage of p24+ CD4 T cells was detected by FACS. Shown are representative data of three independent experiments (mock, n=9; HIV, n=9, HIV-1+cART, n=15 in total) from n=4 (mock), n=3 (HIV-1) or n=7 (HIV-1+cART) hu-mice per group. (C) Humanized mice infected with HIV-1 were treated with cART from 4-10 wpi. Relative mRNA levels of OASland IRF-7 in PBMCs at indicated time points. Unpaired, two-tailed Student's t-test was performed to compare between mock and HIV-1 plus cART group at singular time points. *P<0.05, **P<0.01. Shown are combined data from two independent experiments with mean values±s.e.m. (mock, n=7; HIV-1, n=7; HIV-1+cART, n=8). (D) Cell-associated HIV-1 DNA and relative level of cell-associated HIV-1 RNA to human CD4 mRNA in human cells from spleen was quantified by PCR. (E) Replication-competent HIV-1 viruses from spleen were detected by the quantitative virus outgrowth assay. Shown are representative data (D, E) from n=4 (mock), n=4 (HIV-1) and n=4 (HIV-1+cART) hu-mice per group of two independent experiments. *P<0.05, **P<0.01, ***P<0.001, by one-way analysis of variance (ANOVA) and Bonferroni's post hoc test. (F) Humanized mice infected with HIV-1 were treated with cART from 4 to 10 wpi. cART was discontinued at week 10. HIV-1 RNA levels in the plasma of each mouse were shown. The broken horizontal line in (A) and (F) is the limit of detection of the assay.

FIGS. 2A-2C show the a-IFNaR1 antibody can bind human IFNaR1 and block type I interferon signaling. (A) Histogram shows the binding of anti-human IFNaR1 antibody to 293T cells transfected with plasmid encoding human IFNaR1. mIgG2a was used as isotype control. (B) The IFN-I reporter cell line was stimulated with human IFN-a2b in the present of anti-human IFNaR1 or isotype control mIgG2a antibody. Data show IFN activity after anti-human IFNaR1 treatment relative to samples with IFN-α2a treatment only. The half maximal inhibitory concentration (IC50) is 1.04 μ/ml. (C) Human PBMCs were pre-incubated with anti-human IFNaR1 antibody for 1 hour and then stimulated with human IFNα2b for 5 hours. Data show the relative expression of human ISG15 and Mx2 detected by quantitative RT-PCR.

FIGS. 3A-3B show the anti-human IFNaR1 antibody does not bind to mouse IFNaR1 or IFN-α mediated signaling in mouse cells. (A) 293T cells were transfected with blank plasmid and plasmid encoding mouse IFNaR1, then incubated with anti-human IFNaR1 antibodies to test the binding of the anti-human IFNaR1 antibody to mouse IFNaR1. Anti-mouse IFNaR1 antibody was used as positive control. (B) Splenocytes from mouse were pre-incubated with anti-human IFNaR1 antibody or anti-mouse IFNaR1 antibody for 1 hour and then stimulated with mouse IFNα for 4 hours. Data show the relative expression of mouse ISG15 and Mx2 detected by quantitative RT-PCR.

FIGS. 4A-4B show anti-IFNaR1 mAb efficiently blocks R848 induced ISGs in vivo in humanized mice. (A) Schematic diagram of the experimental design. Humanized mice were pretreated with PBS, isotype control (mouse IgG2a) or α-IFNaR1 antibody (200 μ/mouse, intraperitoneal injection), and 6 hours later, the mice received PBS or R848 (20 μ/mouse, intraperitoneal injection) treatment. At 18 hours, peripheral blood cells were collected for analysis. (B) The relative mRNA levels of human Mx2, ISG15, OAS-1, and IRF7 were detected by real-time PCR. Shown are combined data of two independent experiments (PBS, n=10; R848+mIgG2a, n=9; R848+ α-IFNaR1, n=9) with mean values±s.e.m. *P<0.05, **P<0.01, ***P<0.001. One-way analysis of variance (ANOVA) and Bonferroni's post hoc test.

FIGS. 5A-5C show α-IFNaR1 mAb treatment in vivo does not affect human immune cell percentage and number in humanized mice. Humanized mice were treated with PBS or α-IFNaR1 mAb (200 μg/mouse, i.p.). Mice were sacrificed 24 hours later. The percentage (A) and number (B) of human total immune cells (hCD45+), T cells (hCD45+ CD3+), B cells (hCD45+ CD19+), myeloid DCs (hCD45+ CD3⁻ CD19⁻ C D14⁻ CD11c+), plasmacytoid DCs (hCD45+ CD3⁻ CD19⁻ CD11c⁻ CD14⁻ CD303+) and monocytes (hCD45+ CD14+) in the spleen were shown. (C) Relative mRNA levels of OAS1 and IRF-7 in splenocytes were detected by realtime PCR. Shown are data from one experiment (PBS, n=3; α-IFNaR1, n=3) with mean values±s.e.m.

FIGS. 6A-6G show IFNaR blockade during cART-suppressed HIV-1 infection completely reverses aberrant immune activation. (A) Schematic diagram of the experimental design. Humanized mice infected with HIV-1 were treated with cART from 4-12 weeks post infection (wpi). From 7 to 10 wpi, the cART-treated mice were injected with α-IFNaR1 antibody or isotype control mIgG2a antibody twice a week. (B) Relative mRNA levels of OAS1 and IRF-7 in PBMCs at 9 wpi. (C) Mice were sacrificed at 12 wpi. Summarized data show numbers of human CD8 and CD4 T cells in spleens. (D) Representative dot plots show the percent HLA-DR⁺CD38+ of CD8 T cells from spleens. (E) Summarized data show percent HLA-DR⁺CD38+ of CD8 and CD4 T cells from spleens. (F) Representative dot plots show percent Ki67+ of CD8 T cells from spleens. (G) Summarized data show percent Ki67+ of CD8 and CD4 T cells from spleens. Shown are combined data from two independent experiments with mean values±s.e.m. (mock, n=7; HIV-1, n=7; HIV-1+cART+mIgG2a, n=8; HIV-1+cART+ α-IFNaR1, n=8). *P<0.05, **P<0.01, ***P<0.001, by one-way analysis of variance (ANOVA) and Bonferroni's post hoc test.

FIGS. 7A-7B show cART rescue human immune cell number. Humanized mice were treated as in FIG. 10A. Mice were sacrificed at 12 wpi. (A) Summarized data show numbers of total human leukocytes in spleen. (B) Summarized data show percentage of human CD4 T cells in total human leukocytes in spleen. Shown are combined data from two independent experiments with mean values±s.e.m. (mock, n=7; HIV-1, n=7; HIV-1+cART+mIgG2a, n=8; HIV-1+cART+α-IFNaR1, n=8). *P<0.05, **P<0.01, ***P<0.001. One-way analysis of variance (ANOVA) and Bonferroni's post hoc test.

FIGS. 8A-8E show IFNaR blockade during cART-suppressed HIV-1 infection reverses the exhaustion phenotype of CD8 T cells and restores anti-HIV-1 T cell function. Humanized mice were treated as in FIGS. 6A-6C. (A) Representative dot plots show percent PD-1+ and TIM-3+ of CD8 T cells from spleens. (B) Summarized data show percent PD-1+ and TIM-3+ of CD8 and CD4 T cells from spleens. (C) RNAseq was performed with purified CD8 T cells from spleens. Expression of CD160, TIGIT and BATF in CD8 T cells from mock—(n=2), HIV-1+cART+mIgG2a—(n=3) and HIV-1+cART+α-IFNaR1-treated (n=3) humanized mice. TPM (Transcripts Per kilobase Million) indicates the relative abundance of transcripts. Unpaired, two-tailed Student's t-test was performed to compare between groups (C). (D-E) Splenocytes were stimulated ex vivo with HIV-1 Gag peptide pools for 8 hours (BFA added at 3 hours) followed by intracellular cytokine staining. Representative dot plots (D) and summarized data (E) show percentages of IFN-α and IL-2 producing CD8 T cells. Shown are combined data from two independent experiments (A-B, D-E) with mean values±s.e.m. (mock, n=7; HIV-1, n=7; HIV-1+cART+mIgG2a, n=8; HIV-1+cART+α-IFNaR1, n=8). *P<0.05, **P<0.01, ***P<0.001, by one-way ANOVA and Bonferroni's post hoc test.

FIGS. 9A-9C show IFNaR blockade in cART-suppressed infection reverses HIV-specific T cell function. Humanized mice were treated as in FIG. 10A. Splenocytes were stimulated ex vivo with HIV Gag peptide pools for 8 hours (BFA was added at 3 hours) followed by intracellular cytokine staining (A)Representative dot plot shows IFN-α and IL-2 producing CD4 T cells. (B) Summarized data show percentages of IFN-α and IL-2 producing CD4 T cells after Gag peptide pools stimulation. (C) Mix splenocytes from the mice were stimulated with PMA/Ionomycine as positive control. Representative dot plot shows IFN-α and IL-2 producing CD8 and CD4 T cells 4 hours after PMA/Ionomycine. Shown are combined data (B) from two independent experiments with mean values±s.e.m. (mock, n=7; HIV-1, n=7; HIV-1+cART+mIgG2a, n=8; HIV-1+cART+α-IFNaR1, n=8). *P<0.05, **P<0.01, ***P<0.001. One-way analysis of variance (ANOVA) and Bonferroni's post hoc test.

FIGS. 10A-10D show IFNaR blockade during cART reduces cART-resistant HIV-1 reservoirs. Humanized mice infected with HIV-1 were treated with cART from 4-12 wpi. From 7 to 10 wpi, the cART treated mice were injected with α-IFNaR1 antibody or isotype control mIgG2a antibody. (A) HIV-1 RNA levels in the plasma. The broken horizontal line indicates the limit of detection of the assay (400 copies/ml). (B) Cell-associated HIV-1 DNA in human cells from spleen and bone marrow was quantified by PCR. (C) Relative levels of cell-associated HIV-1 RNA in human cells from spleens and bone marrows were quantified by PCR. (D) Replication-competent HIV-1 viruses from spleens were detected by the quantitative virus outgrowth assay. Shown are combined data from two independent experiments with mean values±s.e.m. (mock, n=7; HIV-1, n=7; HIV-1+cART+mIgG2a, n=8; HIV-1+cART+α-IFNaR1, n=8). *P<0.05, **P<0.01, ***P<0.001, by one-way ANOVA and Bonferroni's post hoc test.

FIGS. 11A-11D show IFNaR blockade during cART delays HIV-1 rebound post-cART cessation. (A) Schematic diagram of the experimental design. Humanized mice infected with HIV-1 for 7-9 weeks were treated with cART. The mice were then injected with α-IFNaR1 or isotype control mIgG2a antibody 5 times (twice a week) starting from week 4 post-cART. cART was maintained for additional 2.5 weeks after the last antibody treatment. Virus rebound was detected by PCR weekly after cART cessation. (B) Plasma HIV-1 viremia in mice treated with cART plus α-IFNaR1 mAb or control mIgG2a. The broken horizontal line indicates the detection limit. (C) Kinetic analysis of HIV-1 rebound after cART cessation. (D) Cell-associated HIV-1 DNA and RNA in PBMCs at 2 weeks post-cART cessation. Shown are combined data of three independent experiments (B, C) (HIV-1+cART+mIgG2a, n=18; HIV-1+cART+α-IFNaR1, n=11) or two independent experiments (D) (HIV-1+cART+mIgG2a, n=10; HIV-1+cART+α-IFNaR1, n=7) with mean values±s.e.m. *P<0.05. Gehan-Breslow-Wilcoxon Test (C) or unpaired, two-tailed Student's t-test (D) was performed.

FIGS. 12A-12D show HIV-1 persistent infection in humanized mice leads to sustained and systemic IFN-I expression and ISGs induction. Humanized mice were infected with HIV-1. (A) Plasma HIV-1 genomic RNA levels at indicated time points. (B) Human IFN-α (pan IFN-a) levels in plasma at indicated time points and IFN-β level in plasma at 10.5 weeks post infection (wpi). (C) Relative mRNA levels of Mx2, IFITM3, Trim22, ISG15, OAS1, MxA and IRF7 in PBMCs at indicated time points. (D) The relative mRNA levels of Mx2, IFITM3, Trim22, ISG15, OAS1, MxA and IRF7 in spleens at termination (10.5 wpi). Shown are representative data (mock, n=4; HIV-1, n=5) of four independent experiments (mock, n=12; HIV-1, n=18 in total) with mean values±s.e.m. *P<0.05, **P<0.01, ***P<0.001. Unpaired, two-tailed Student's t-test was performed to compare between groups at singular time points (B, C) or between two groups (D).

FIGS. 13A-13I show HIV-1 persistent infection leads to human T cell depletion and hyper-immune activation. Humanized mice were infected with HIV-1 and analyzed at indicated weeks post-infection (wpi). (A) Percentage of CD4 T cells in total T cells and number of CD4 T cells in peripheral blood during HIV-1 infection at indicated time points. (B) Percentage of HLA-DR+/CD38+ CD8 T cells in peripheral blood at indicated time points. (C-E) Number of human CD4 T cells (C), CD8 T cells (D) and total human CD45+ cells (E) in spleen and mLNs at termination (10.5 wpi). (F) Representative FACS plot show expression of CD38/HLA-DR and Ki67 on CD8 T cells. (G-II) Summarized data show expression of CD38/HLA-DR and Ki67 on CD8 T cells and CD4 T cell in the spleen at termination (10.5 wpi). (I) Summarized data show expression of CD38/HLA-DR on CD8 and CD4 T cell in the mesenteric lymph nodes (mLNs) at termination(10.5 wpi. Shown are representative data of four independent experiments (mock, n=12; HIV-1, n=18 in total) with mean values±s.e.m. from n=4 (mock) or n=5 (HIV-1) hu-mice per group. *P<0.05, **P<0.01, ***P<0.001. Unpaired, two-tailed Student's t-test was performed to compare between groups at singular time point (A, B) or between two groups (C-I).

FIGS. 14A-14C show HIV-1 persistent infection in humanized mice leads to impaired T cell function. Humanized mice were infected with HIV-1 and terminated at week 10.5. (A) Representative dot plots and summarized data show percent PD-1+ and TIM-3+ of CD8 T cells in spleens. (B, C) At 10.5 wpi, splenocytes were stimulated ex vivo with PMA/ionomycin for 4 hours followed by intracellular cytokine staining. (B) Representative dot plots (of cells gated on human CD45+CD3+CD8+) and summarized data show percentages of IFN-γ and IL-2 producing CD8 T cells. (C) Representative dot plots (of cells gated on human CD45+CD3+CD4+) and summarized data show percentages of IFN-α and IL-2 producing CD4 T cells. Shown are representative data (mock, n=4; HIV-1, n=5) of four independent experiments (mock, n=12; HIV-1, n=18 in total) with mean values s.e.m. *P<0.05, **P<0.01, ***P<0.001. Unpaired, two-tailed Student's t-test was performed.

FIGS. 15A-15E show IFNAR1 blockade during persistent HIV-1 infection enhances viral replication in humanized mice. Humanized mice infected with HIV-1 were treated from 6-10 wpi with α-IFNAR1 mAb or isotype control (mouse IgG2a) twice a week. (A) Relative mRNA levels of human ISGs including Mx2, IFITM3, TRIM22 and IRF7 in PBMCs at 9 wpi. (B) Plasma HIV-1 RNA levels at indicated time points after HIV-1 infection. (C) Human IFN-α levels in the plasma after HIV-1 infection. Shown (A-C) are representative data (mock, n=3; HIV-1+mIgG2a, n=5; HIV-1+α-IFNAR1, n=5) of three independent experiments (mock, n=7; HIV-1+mIgG2a, n=11; HIV-1+α-IFNAR1, n=12 in total) with mean values±s.e.m. (D) Relative mRNA levels of human Mx2, IFITM3, TRIM22 and IRF7 in splenocytes at 10 wpi. (E) Representative FACS plots and summarized data show percentages of HIV-1 p24-positive CD4 T cells (CD3+CD8⁻) in the spleen at 10 wpi. Shown (D-E) are combined data of two independent experiments (mock, n=6; HIV-1+mIgG2a, n=9; HIV-1+α-IFNAR1, n=9) with mean values±s.e.m. *P<0.05, **P<0.01, ***P<0.001. One-way analysis of variance (ANOVA) and Bonferroni's post hoc test were performed to compare between groups at singular time points (B, C) or between groups (A, D, E).

FIGS. 16A-16C show IFNAR1 blockade reduces ISGs expression during persistent HIV-1 infection in humanized mice. Humanized mice infected with HIV-1 were treated with α-IFNAR1 mAb or isotype control (mIgG2a) twice a week from 6 to 10 wpi. (A, B) The relative mRNA level of indicated ISGs expression in PBMCs at week 7 (A) and week 9 (B). Shown are representative data of three independent experiments (mock, n=7; HIV-1+mIgG2a, n=11; HIV-1+α-IFNAR1, n=12 in total) with mean values±s.e.m. from n=3 (Mock), n=5 (HIV-1+mIgG2a) or n=5 (HIV-1+α-IFNAR1) hu-mice per group. (C) The relative mRNA expression level of indicated ISGs and in spleen at termination (10.5 wpi). Shown are combined data of two independent experiments (mock, n=6; HIV-1+mIgG2a, n=9; HIV-1+α-IFNAR1, n=9) with mean values±s.e.m. *P<0.05, **P<0.01, ***P<0.001. One-way analysis of variance (ANOVA) and Bonferroni's post hoc test was performed.

FIGS. 17A-17E show IFNAR1 blockade during persistent HIV-1 infection increases expression of HLA-DR/CD38 and Ki67 on T cells. Humanized mice infected with HIV-1 were treated from 6-10 wpi with α-IFNAR1 mAb or isotype control (mouse IgG2a) twice a week. Mice were sacrificed at 10 wpi. (A) Summarized data show CD8 and CD4 T cells expressing HLA-DR/CD38 (mock, n=9; HIV-1+mIgG2a, n=12; HIV-1+α-IFNAR1, n=12, combined data from 3 independent experiment with mean values±s.e.m.). (B) Summarized data show CD8 and CD4 T cells expressing Ki67 (mock, n=6; HIV-1+mIgG2a, n=8; HIV-1+α-IFNAR1, n=8, combined data from 2 independent experiments with mean values±s.e.m.). (C) Correlation analysis between HLA-DR/CD38 expression on T cells and plasma HIV-1 RNA levels. (D) Correlation analysis between Ki67 expression on T cells and plasma HIV-1 RNA levels. (E) The levels of indicated cytokines in the plasma of hu-mice at 10 wpi. Shown (E) are combined data of 3 independent experiments (mock, n=6; HIV-1+mIgG2a, n=9; HIV-1+α-IFNAR1, n=11) with mean values±s.e.m. *P<0.05, **P<0.01, ***P<0.001. One-way analysis of variance (ANOVA) and Bonferroni's post hoc test (A, B, E) and Spearman rank correlation test (C, D) were performed.

FIGS. 18A-18D show IFNAR1 blockade during persistent HIV-1 infection rescues human T cells and total human leukocytes in humanized mice. Humanized mice infected with HIV-1 were treated with α-IFNAR1 mAb or isotype control (mouse IgG2a) twice a week from 6-10 wpi. Mice were sacrificed at 10 wpi. (A-B) Numbers of CD4 T cells (CD3+CD8⁻), CD8 T cells (CD3+CD8⁻) and total human leukocytes in spleens (A) (mock, n=13; HIV-1+mIgG2a, n=18; HIV-1+α-IFNAR1, n=17, combined data from 5 independent experiments) and mesenteric lymph nodes (mLNs) (B) (mock, n=8; HIV-1+mIgG2a, n=12; HIV-1+α-IFNAR1, n=11, combined data from 3 independent experiments with mean values±s.e.m.). (C-D) Humanized NSG-A2 mice transplanted with HSCs from HLA-A2 matched donor were infected with HIV-1 and treated with α-IFNAR1 mAb or isotype control (mouse IgG2a) twice a week from 6-10 wpi. Mice were sacrificed at 10 wpi. Representative dot plots (C) and summarized data (D) show percentages of CD8 T cells specific for the HLA-A2/SL-9 pentamer (an HLA-A2 restricted epitope consisting of amino acids 77-85 of HIV-1 p17 protein) among CD8 T cells from LNs (mock, n=3; HIV-1+mIgG2a, n=3; HIV-1+α-IFNAR1, n=3) and spleens (mock, n=3; HIV-1+mIgG2a, n=4; HIV-1+α-IFNAR1, n=4). *P<0.05, **P<0.01, ***P<0.001. One-way analysis of variance (ANOVA) and Bonferroni's post hoc test was performed.

FIGS. 19A-19B show IFNAR1 blockade rescues human HIV-specific CD8 T cell number during persistent HIV-1 infection in humanized mice. Humanized mice were treated as in FIG. 18C. Absolute number of CD8 T cells specific for the HLA-A2/SL-9 pentamer in LNs (mock, n=3; HIV-1+mIgG2a, n=3; HIV-1+α-IFNAR, n=3) and spleens (mock, n=3; HIV-1+mIgG2a, n=4; HIV-1+α-IFNAR1, n=4). *P<0.05 by unpaired, two-tailed Student's t-test.

FIGS. 20A-20I show IFNAR1 blockade or inhibition of caspase-3 activity rescues CD4 T cells from HIV-1 induced depletion. (A) Humanized mice infected with HIV-1 were treated with α-IFNAR1 mAb or isotype control (mouse IgG2a) twice a week from 6-10 wpi. Mice were sacrificed at 10 wpi. Representative dot plots and summarized data (mock, n=6; HIV-1+mIgG2a, n=9; HIV-1+α-IFNAR1, n=9, combined data from 2 independent experiment with mean values±s.e.m.) show percent splenic CD4 T cells expressing active caspase-3. (B-E) Splenocytes from mock (n=2) or HIV-1 infected (n=4) humanized mice were cultured with IL-2 (20 u/ml) in vitro in the presence of control mIgG2a (10 μg/ml), α-IFNAR1 (10 μg/ml) or IFN-α (200 u/ml) for 10 days. At day 10, the cells were counted and used for analysis. (B) HIV-1 RNA levels in culture supernatant. (C-D) Representative dot plots (C) and summarized data (D) show percentages of CD4 T cells with active caspase-3. (E) Number of live CD4 T cells in each group. (F-I) Splenocytes from mock (n=2) or HIV-1 infected (n=4) humanized mice were cultured with IL-2 in vitro in the presence of DMSO or caspase-3 inhibitor Z-DEVD-FMK or caspase-1 inhibitor Ac-YVAD-CMK for 10 days. At day 10, the cells were counted and used for staining. (F-G) Representative dot plots (F) and summarized data (G) show percentages of active caspase-3⁺ CD4 T cells. (H-I) Number of live CD4 T cells in each group. Data are one representative of 2 independent experiments with mean values±s.e.m.). *P<0.05, **P<0.01 by unpaired, two-tailed Student's t-test to compare differences between each two groups.

FIGS. 21A-21B show detection of activated caspase-1 in HIV-1 infected samples. Splenocytes from mock (n=2) or HIV-1 infected (n=4) humanized mice were cultured with IL-2 (20 u/ml) in vitro in the presence of control mIgG2a (10 μg/ml), α-IFNAR (10 μg/ml) or IFN-α (200 u/ml) for 10 days. At day 10, the cells were used for staining. Representative dot plots (A) and summarized data (B) show percentage of CD4 T cells with active caspase-1.

FIGS. 22A-22F show IFNAR1 blockade during persistent HIV-1 infection rescues the function of human T cells, including HIV-specific T cells. Humanized mice infected with HIV-lwere treated with α-IFNAR1 mAb or isotype control (mouse IgG2a) twice a week from 6-10 wpi. Mice were sacrificed at 10 wpi. (A-C) Splenocytes were stimulated ex vivo with PMA plus ionomycin for 4 hours followed by intracellular cytokine staining. Representative dot plots (A) and summarized data show percentages of CD8 T cells (B) and CD4 T cells (C) producing IFN-γ (mock, n=11; HIV-1+mIgG2a, n=15; HIV-1+α-IFNAR1, n=13, combined data from 4 independent experiments with mean values±s.e.m.) or IL-2 (mock, n=8; HIV-1+mIgG2a, n=10; HIV-1+α-IFNAR1, n=10, combined data from 3 independent experiments with mean values±s.e.m.). (D-F) Splenocytes were stimulated ex vivo with peptide pools of HIV-1 Gag protein for 8 hours (Brefeldin A was added at 3 hours) followed by intracellular cytokine staining. Representative dot plots (D) and summarized data show percentages of CD8 T cells (E) and CD4 T cells (F) producing IFN-α and IL-2 (mock, n=4; HIV-1+mIgG2a, n=6; HIV-1+α-IFNAR1, n=6, combined data from 2 independent experiment with mean values±s.e.m.). *P<0.05, **P<0.01, ***P<0.001 by one-way analysis of variance (ANOVA) and Bonferroni's post hoc test.

FIGS. 23A-23B show IFNAR1 blockade during persistent HIV-1 infection rescues the function of human CD4 T cells. Humanized mice infected with HIV-1 were treated with α-IFNAR1 mAb or isotype control (mouse IgG2a) twice a week from 6-10 wpi. Mice were sacrificed at 10 wpi. (A) Splenocytes were stimulated ex vivo with PMA plus ionomycin for 4 hours followed by intracellular cytokine staining. Representative dot plots show percentages of IFN-α and IL-2 producing CD4 T cells. (B) Splenocytes were stimulated ex vivo with peptide pool of HIV Gag protein for 8 hours (Brefeldin A was added at 3 hours) followed by intracellular cytokine staining. Representative dot plots show percentages of IFN-γ and IL-2 producing CD4 T cells.

FIGS. 24A-24B show detection of PD-1 expression on CD8 T cells after IFNAR1 blockade. Humanized mice infected with HIV-1 were treated with α-IFNAR1 mAb or isotype control (mouse IgG2a) twice a week from 6-10 wpi. Mice were sacrificed at 10 wpi. (A) Summarized data show PD-1 expressing CD8 T cells from the spleen (mock, n=6; HIV-1+mIgG2a, n=7; HIV-1+α-IFNAR1, n=7, combined data from 2 independent experiment with mean values±s.e.m.). *P<0.05, one-way analysis of variance (ANOVA) and Bonferroni's post hoc test was performed. (B) Correlation analysis between PD-1 expression on CD8 T cells and plasma HIV-1 RNA levels. Spearman rank correlation test was performed.

FIGS. 25A-25E show CD8 T cells are required for IFNAR blockade to reduce HIV-1 reservoir. Hu-mice infected with HIV-1 were treated with cART from 4-13 wpi. From 7 to 10 wpi, cART treated mice were injected with α-IFNAR bAb, CD8 depletion Ab or isotype control. (A) HIV-1 plasma viremia. (B) Specific depletion of CD8 T cells in spleen. (C) HIV viremia in the blood during infection and treatments. (D) HIV reservoirs as determined by cell-associated HIV DNA. (E) HIV reservoirs with replication-competent HIV-1 viruses were detected by virus outgrowth assay (VOA). (mock, n=3; HIV-1, n=3; HIV-1+cART+iso, n=5; HIV-1+cART+IFNaR bAb, n=5). **P<0.01.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

All publications, patent applications, patents, patent publications and other references cited herein are incorporated by reference in their entireties for the teachings relevant to the sentence and/or paragraph in which the reference is presented.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

The transitional phrase “consisting essentially of” means that the scope of a claim is to be interpreted to encompass the specified materials or steps recited in the claim, and those that do not materially affect the basic and novel characteristics of the claimed invention.

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

The term “reactivate,” as used herein, refers to the activation of latent HIV-1 proviruses present in resting CD4⁺ T cells.

The term “latent,” as used herein, refers to replication competent HIV-1 proviruses present in resting CD4⁺ T cells.

The term “reservoir,” as used herein, refers to the latent but replication competent HIV-1 proviruses present in resting CD4⁺ T cells.

The term “type I interferon signaling,” as used herein, refers to the signaling pathway modulated by the binding of interferon-1 to the interferon-α/β receptor.

The term “immune hyperactivation,” as used herein, refers to the expression of immune activation markers (CD38, HLA-DR and Ki67) on human T cells, interferon-stimulated genes (ISG) and of inflammatory cytokines.

The term “non-responder,” as used herein, refers to a subject that exhibits no response or a non-therapeutic response to a therapeutic treatment such as cART.

An “effective” amount as used herein is an amount that provides a desired effect.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

By the terms “treat,” “treating,” or “treatment,” it is intended that the severity of the subject's condition is reduced or at least partially improved or modified and that some alleviation, mitigation or decrease in at least one clinical symptom is achieved.

One aspect of the invention relates to a method of reactivating latent HIV-1 in a subject in need thereof, comprising inhibiting type I interferon signaling in the subject, thereby reactivating latent HIV-1 in the subject.

Another aspect of the invention relates to a method of reducing HIV-1 reservoirs in a subject in need thereof, comprising inhibiting type I interferon signaling in the subject, thereby reducing HIV-1 reservoirs in the subject.

A further aspect of the invention relates to a method of treating HIV-1 infection in a subject in need thereof, comprising inhibiting type I interferon signaling in the subject, thereby treating HIV-1 infection in the subject.

An additional aspect of the invention relates to a method of increasing the effectiveness of combination antiretroviral therapy (cART) for HIV-1 infection in a subject in need thereof, comprising inhibiting type I interferon signaling in the subject prior to, during, and/or after cART, thereby increasing the effectiveness of cART in the subject.

Another aspect of the invention relates to a method of inhibiting immune hyperactivation in a subject in need thereof, comprising inhibiting interferon-I signaling in the subject, thereby inhibiting immune hyperactivation in the subject.

In each of the methods of the invention, inhibiting interferon-I signaling may be carried out by any method known in the art and as described herein. Interferon-I signaling may be inhibited by interfering with the function of interferon-I, one or more receptors to which interferon-I binds, or both. In some embodiments, inhibiting interferon-I signaling comprises delivering to the subject an effective amount of an antibody or a fragment thereof that specifically binds to the interferon-α/β receptor.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, goat, sheep, camel, or human, or can be a chimeric antibody. See, e.g., Walker et al., Molec. Immunol. 26:403 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, Fab′, F(ab′)₂, and Fv fragments; domain antibodies, diabodies; vaccibodies, linear antibodies; single-chain antibody molecules (e.g., scFv); and multispecific antibodies formed from antibody fragments. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively, Fab expression libraries can be constructed to allow rapid and easy identification of monoclonal Fab fragments with the desired specificity (Huse et al., Science 254:1275 (1989)).

Antibodies of the invention may be altered or mutated for compatibility with species other than the species in which the antibody was produced. For example, antibodies may be humanized or camelized. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody optimally also will comprise at least a portion of an immunoglobulin constant region (Fc), typically that of a human immunoglobulin (Jones et al., Nature 321:522 (1986); Riechmann et al., Nature, 332:323 (1988); and Presta, Curr. Op. Struct. Biol. 2:593 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain Humanization can essentially be performed following the method of Winter and co-workers (Jones et al., Nature 321:522 (1986); Riechmann et al., Nature 332:323 (1988); Verhoeyen et al., Science 239:1534 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues (e.g., all of the CDRs or a portion thereof) and possibly some FR residues are substituted by residues from analogous sites in rodent antibodies.

Human antibodies can also be produced using various techniques known in the art, including phage display libraries (Hoogenboom and Winter, J. Mol. Biol. 227:381 (1991); Marks et al., J Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147:86 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779 (1992); Lonberg et al., Nature 368:856 (1994); Morrison, Nature 368:812 (1994); Fishwild et al., Nature Biotechnol. 14:845 (1996); Neuberger, Nature Biotechnol. 14:826 (1996); Lonberg and Huszar, Intern. Rev. Immunol. 13:65 (1995).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies used to carry out the present invention can be produced in a hybridoma cell line according to the technique of Kohler and Milstein, Nature 265:495 (1975). For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., Huse, Science 246:1275 (1989).

Antibodies specific to the target polypeptide can also be obtained by phage display techniques known in the art.

Various immunoassays can be used for screening to identify antibodies having the desired specificity for the target polypeptide. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes on the target polypeptide can be used as well as a competitive binding assay.

Antibodies can be conjugated to a solid support (e.g., beads, plates, slides or wells formed from materials such as latex or polystyrene) in accordance with known techniques. Antibodies can likewise be conjugated to detectable groups such as radiolabels (e.g., ³⁵S, ¹²⁵I, ₁₃₁I), enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

In each of the methods of the invention, interferon-I signaling may be inhibited to the extent necessary to achieve the goals of the methods. In some embodiments, interferon-I signaling may be inhibited by at least about 50%, e.g., at least about 50%, 60%, 70%, 80%, 90%, 95%, or more. The inhibition may be carried out for a suitable length of time to achieve the goals of the methods.

In some embodiments of the methods of the invention, the subject is a non-responder to cART. The methods of the present invention may be used to enhance the effectiveness of cART in non-responders. In certain embodiments, the subject previously underwent cART. The methods of the present invention may be used to help control HIV rebound after cART interruption.

In certain embodiments, the methods of the invention may further comprise delivering to the subject one or more HIV-1 therapeutic agents. The one or more HIV-1 therapeutic agents may be any agent or combination of agents known to be effective for the treatment of HIV-1 infection. In certain embodiments, the HIV-1 therapeutic agent is an antiretroviral agent. Examples of antiretroviral agents include, without limitation, reverse transcriptase inhibitors, protease inhibitors, viral integration inhibitors, viral entry inhibitors, viral maturation inhibitors, iRNA agents, antisense RNA, vectors expressing iRNA agents or antisense RNA, PNA, antiviral antibodies and any combination thereof. See for example, US Pat. No. 8,497,251, incorporated by reference in its entirety.

In some embodiments, the antiretroviral agent is selected from the group consisting of AZT, 3TC, ddI, ddC, 3TC, saquinavir, indinavir, ritonavir, nelfinavir, nevirapine, efavirenz, and combinations thereof.

In some embodiments, the HIV-1 therapeutic agent is an antibody or a fragment thereof that specifically binds to BDCA2 and depletes plasmacytoid dendritic cells.

In some embodiments, the methods of the invention further comprise delivering to the subject a combination or cocktail of HIV-1 therapeutic agents as is known in the art, such as combination anti-retroviral therapy (cART) or highly active antiretroviral therapy (HAART), e.g., at least two or three different drugs from at least two different classes selected from reverse transcriptase inhibitors, protease inhibitors, viral integration inhibitors, viral entry inhibitors, and viral maturation inhibitors.

In some embodiments, the subject is a subject in need of treatment, e.g., a subject that has or is suspected of having an HIV-1 infection or has been diagnosed with a disease or disorder associated with HIV-1 infection, e.g., ARC or AIDS. In some embodiments, the subject is a human. In some embodiments, the subject is an animal model of HIV-1 infection, e.g., a rodent such as a mouse or a primate such as a monkey.

As a further aspect, the invention provides pharmaceutical formulations and methods of administering the same to carry out the methods of the invention. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The compounds of the invention can be formulated for administration in a pharmaceutical carrier in accordance with known techniques. See, e.g., Remington, The Science And Practice of Pharmacy (21^{th} Ed. 2005). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

A further aspect of the invention is a method of treating subjects in vivo, comprising administering to a subject a pharmaceutical composition comprising a compound of the invention in a pharmaceutically acceptable carrier, wherein the pharmaceutical composition is administered in a therapeutically effective amount. Administration of the compounds of the present invention to a human subject or an animal in need thereof can be by any means known in the art for administering compounds.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into the brain for delivery to the central nervous system, into the pancreas, or into a tumor or the tissue surrounding a tumor). In some embodiments, the formulation is delivered to the site of tissue damage (e.g., fibrosis) or inflammation. The most suitable route in any given case will depend on the nature and severity of the condition being treated and on the nature of the particular compound which is being used.

In certain embodiments, the inhibitor is administered via one or more of oral administration, injection, and a surgically implanted pump. In some embodiments, the administration is via intravenous injection, intraportal delivery, or direct liver injection.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein. The technology for forming liposomal suspensions is well known in the art. When the compound is in the form of an aqueous-soluble material, using conventional liposome technology, the same can be incorporated into lipid vesicles. In such an instance, due to the water solubility of the compound, the compound will be substantially entrained within the hydrophilic center or core of the liposomes. The lipid layer employed can be of any conventional composition and can either contain cholesterol or can be cholesterol-free. When the compound of interest is water-insoluble, again employing conventional liposome formation technology, the compound can be substantially entrained within the hydrophobic lipid bilayer which forms the structure of the liposome. In either instance, the liposomes which are produced can be reduced in size, as through the use of standard sonication and homogenization techniques. The liposomal formulations containing the compound disclosed herein, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In addition to the compound, the pharmaceutical compositions can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions can contain microbial preservatives. Useful microbial preservatives include methylparaben, propylparaben, and benzyl alcohol. The microbial preservative is typically employed when the formulation is placed in a vial designed for multidose use. Other additives that are well known in the art include, e.g., detackifiers, anti-foaming agents, antioxidants (e.g., ascorbyl palmitate, butyl hydroxy anisole (BHA), butyl hydroxy toluene (BHT) and tocopherols, e.g., α-tocopherol (vitamin E)), preservatives, chelating agents (e.g., EDTA and/or EGTA), viscomodulators, tonicifiers (e.g., a sugar such as sucrose, lactose, and/or mannitol), flavorants, colorants, odorants, opacifiers, suspending agents, binders, fillers, plasticizers, lubricants, and mixtures thereof. The amounts of such additives can be readily determined by one skilled in the art, according to the particular properties desired.

The additive can also comprise a thickening agent. Suitable thickening agents can be those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products. Such thickening agents as described above can be included, e.g., to provide a sustained release effect. However, where oral administration is intended, the use of thickening agents as aforesaid will generally not be required and is generally less preferred. Use of thickening agents is, on the other hand, indicated, e.g., where topical application is foreseen.

In particular embodiments, the compound is administered to the subject in a therapeutically effective amount, as that term is defined above. Dosages of pharmaceutically active compounds can be determined by methods known in the art, see, e.g., Remington, The Science And Practice of Pharmacy (21^{th} Ed. 2005). The therapeutically effective dosage of any specific compound will vary somewhat from compound to compound, and patient to patient, and will depend upon the condition of the patient and the route of delivery. In one embodiment, the compound is administered at a dose of about 0.001 to about 10 mg/kg body weight, e.g., about 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, or 10 mg/kg. In some instances, the dose can be even lower, e.g., as low as 0.0005 or 0.0001 mg/kg or lower. In some instances, the dose can be even higher, e.g., as high as 20, 50, 100, 500, or 1000 mg/kg or higher. The present invention encompasses every sub-range within the cited ranges and amounts.

A further aspect of the invention relates to the use of animal models of HIV-1 infection in which type I interferon signaling has been inhibited to screen for compounds suitable for treatment of HIV-1 infection. This method takes advantage of a model in which latent HIV-1 has been or can be reduced, permitting an analysis of the efficacy of a compound under these desirable conditions.

Thus, a further aspect of the invention relates to a method for identifying a compound suitable for treatment of HIV-1 infection, the method comprising providing an animal model of HIV-1 infection in which type I interferon signaling has been inhibited, delivering a candidate compound to the animal, and measuring HIV-1 levels in the animal, wherein a decrease in HIV-1 levels compared to an animal that has not received the candidate compound identifies the candidate compound as a compound suitable for treatment of HIV-1 infection.

The type I interferon signaling may be inhibited by any of the methods described above. The level of inhibition of type I interferon signaling may be at least about 50%, e.g., at least about 50%, 60%, 70%, 80%, 90%, 95%, or more.

In some embodiments, the animal is a mouse. In some embodiments, the animal model is a humanized mouse model.

Anti-HIV activity of a therapeutic agent may be measured by any method known in the art. Any in vitro or in vivo assay known in the art to measure HIV infection, production, replication or transcription can be used to test the efficacy of a therapeutic of the invention. For example, but not by way of limitation, viral infection assays, CAT or other reporter gene transcription assays, HIV infection assays, or assays for viral production from cells latently infected with HIV (for example, but not limited to, by the method described by Chun et al., Nature 387:183-188 (1977)) can be used to screen for and test potential inhibitors the virus.

The compounds that may be tested in the model may be a wide range of molecules and is not a limiting aspect of the invention. Compounds include, for instance, a polyketide, a non-ribosomal peptide, a polypeptide, a polynucleotide (for instance an siRNA, antisense oligonucleotide or ribozyme), other organic molecules, or a combination thereof. The sources for compounds to be screened can include, for example, chemical compound libraries, fermentation media of Streptomycetes, other bacteria and fungi, and extracts of eukaryotic or prokaryotic cells.

The present invention is more particularly described in the following examples that are intended as illustrative only since numerous modifications and variations therein will be apparent to those skilled in the art.

### EXAMPLE 1

### Experimental Procedures

Construction of humanized mice. NRG (NOD-Rag2^{-/-}γc^{-/-}) mice were obtained from the Jackson Laboratory. All mice were housed and bred in a specific pathogen-free environment. Humanized NRG mice with a functional human immune system were generated by intrahepatic injection of new born mice with human fetal liver derived CD34⁺ hematopoietic progenitor cells as previously reported (Li et al., PLoS Pathog. 10:e1004291 (2014)). Humanized BLT (bone marrow/liver/thymus) mice were generated as previously reported (Namikawa et al., Science 242:1684 (1988)). Briefly, 6 to 8 weeks old NRG mice were sub-lethally irradiated and anesthetized, and ˜1-mm³ fragments of human fetal thymus were implanted under the kidney capsule. CD34⁺ hematopoietic progenitor cells purified from fetal liver of the same donor were injected i.v. within 3 hours. Human immune cell engraftment was detected by flow cytometry 12 weeks after transplantation. All animal studies were approved by the University of North Carolina Institutional Animal Care and Use Committee (IACUC ID: 14-100).

HIV-1 infection of humanized mice. The R5 tropic strain of HIV-1(JR-CSF) was generated by transfection of 293T cells with plasmid containing full length HIV-1 (JR-CSF) genome. Humanized mice with stable human leukocyte reconstitution were anesthetized and infected with HIV-1 (JR-CSF) (10 ng p24 per mouse which equals to 3000 infectious unit per mouse) through retro-orbital injection. Humanized mice infected with 293T supernatant were used as mock control groups. Both male and female mice were used for all the experiments.

Development of anti-IFNaR1 blocking antibody. The mouse cell line L-929 transfected with the human IFNaR1(extracellular domain and transmembrane domain) expression plasmid mentioned was used as the immunogen for immunization. For each immunization, the wild type BALB/c female mice were injected intraperitoneally with 5000,000 immunogen cells with 10 μg CpG1826 as adjuvant. After 5 times immunization, the spleen cells were fused with mouse myeloma cell line SP2/0. 293 T cells transfected with the human IFNAR1 expression plasmid were used for screening the clones that could secret the IFNAR1 binding antibody by flow cytometry. Briefly, the human IFNAR1 expression 293 T cell line was firstly incubated with the supernatant of the hybridoma, then incubated with the PE labeled goat anti-mouse IgG secondary antibody. Then, a IFN-I reporter 293T cell line which has been stably transfected with an interferon stimulated gene Mx2 promoter driven EGFP was used to screen antibody clones that could block the human IFNAR1 signaling.

In vitro blocking assay. The IFN-I reporter 293T cell line or human PBMCs or mouse splenocytes were pre- incubated with antibodies for 1 hour at 37° C., the human IFNα2b or mouse IFNα was added with a final concentration of 5 ng/ml. IFN-I reporter 293T cells were harvested and GFP expression was analyzed by flow cytometry 24 hours later. The IFN activity after anti-human IFNaR1 relative to samples with IFN-α2a treatment only was calculated. To detect ISGs expression in human PBMCs or mouse splenocytes, cells were harvested 4-5 hours later for ISGs detection by quantitative real-time PCR. The primers used for the quantitative real-time PCR in the in vitro assay were as following:

| | |
|---|---|
| human ISG15 | |
| (5′-CGCAGATCACCCAGAAGATCG-3′ | (SEQ ID NO: 1) |
| and | |
| 5′-TTCGTCGCATTTGTCCACCA-3′, | (SEQ ID NO: 2)) |

human Mx2

| | |
|---|---|
| (5′-CAGAGGCAGCGGAATCGTAA-3′ | (SEQ ID NO: 3) |
| and | |
| 5′-TGAAGCTCTAGCTCGGTGTTC-3′, | (SEQ ID NO: 4)) |

human EF-1α

| | |
|---|---|
| (5′-ATATGGTTCCTGGCAAGCCC-3′ | (SEQ ID NO: 5) |
| and | |
| 5′-GTGGGGTGGCAGGTATTAGG-3′, | (SEQ ID NO: 6)) |

mouse ISG15

| | |
|---|---|
| (5′-TGGTACAGAACTGCAGCGAG-3′ | (SEQ ID NO: 7) |
| and | |
| 5′-AGCCAGAACTGGTCTTCGTG-3′, | (SEQ ID NO: 8)) |

mouse Mx2

| | |
|---|---|
| (5′-GTGGCAGAGGGAGAATGTCG-3′ | (SEQ ID NO: 9) |
| and | |
| 5′-TAAAACAGCATAACCTTTTGCGA-3′, | (SEQ ID NO: 10)) |

mouse GAPDH ISG15

| | |
|---|---|
| (5′-GAGCCAACGGGTCATCT-3′ | (SEQ ID NO: 11) |
| and | |
| 5′-GAGGGGCCATCCACAGTCTT-3′. | (SEQ ID NO: 12)) |

In vivo IFNaR blocking antibody treatments. To confirm the in vivo neutralizing activity of α-IFNaR1 mAbs, humanized mice were treated i.p. with α-IFNaR1mAb or mIgG2a as isotype control 6 hours prior to R848 treatment. HIV-1 infected, cART treated mice were treated i.p. with IFNaR1 blocking antibodies from 7 to 10 wpi twice a week with 400 μg/mouse at the first injection and 200 μg/mouse for the following treatments. A same dose of mouse isotype IgG2a control was use in all experiments. Cohorts of mice were randomized into different treatment groups by level of HIV-1 RNA in plasma.

Quantification of mRNA expression in humanized mice by RT-PCR. RNA from PBMCs or whole splenocytes from humanized mice was isolated with the RNeasy plus extraction kit (Qiagen) and converted to cDNA by reverse transcription with random hexamers and SuperScript® III First-Strand Synthesis (Invitrogen). cDNA was then subjected to quantitative real-time PCR using human gene-specific primers for:

| ISG15 | |
|---|---|
| (5′-CGCAGATCACCCAGAAGATCG-3′ | (SEQ ID NO: 1) |
| and | |
| 5′-TTCGTCGCATTTGTCCACCA-3′, | (SEQ ID NO: 2)) |
| OAS1 | |
| (5′-TGTCCAAGGTGGTAAAGGGTG-3′ | (SEQ ID NO: 13) |
| and | |
| 5′-CCGGCGATTTAACTGATCCTG-3′, | (SEQ ID NO: 14)) |
| Mx2 | |
| (5′-CAGAGGCAGCGGAATCGTAA-3′ | (SEQ ID NO: 3) |
| and | |
| 5′-TGAAGCTCTAGCTCGGTGTTC-3′, | (SEQ ID NO: 4)) |
| IRF7 | |
| (5′-GCTGGACGTGACCATCATGTA-3′ | (SEQ ID NO: 15) |
| and | |
| 5′-GGGCCGTATAGGAACGTGC-3′. | (SEQ ID NO: 16)) |

mRNA from spleen of non-humanized NRG mice treated with the TLR-8 agonist R848 were used as control to test the human specify of the primers. No signal was detected by real-time PCR when mRNA from spleen of non-humanized NRG mice with R848 treatment was used as control. The IFN-stimulated gene expression levels were normalized to mRNA level of human CD45. Results were calculated as a fold change in gene expression, relative to mock mice using the delta-delta Ct method of analysis (Schmittgen et al., Nat. Protoc. 3:1101 (2008)). Briefly, the fold change is calculated by 2^{-ΔΔCt}. ΔΔCt=(Ct gene of interest−Ct internal control)sample of treated mouse−(Ct gene of interest−Ct internal control)sample of mock mouse.

Combination antiretroviral therapy (cART). Food formulated with anti-retroviral individual drug was prepared as reported with elevated dose modifications (Halper-Stromberg et al., Cell 158:989 (2014)). In brief, tablets of emtricitabine and tenofovir disoproxil fumarate (Truvada®; Gilead Sciences) and raltegravir (Isentress®; Merck) were crushed into fine powder and manufactured with TestDiet 5B1Q feed (Modified LabDiet 5058 with 0.12% amoxicillin) into ½″ irradiated pellets. Final concentrations of drugs in the food were 4800 mg/kg raltegravir, 1560 mg/kg tenofovir disoproxil and 1040 mg/kg emtricitabine. The estimated drug daily doses were 768 mg/kg raltegravir, 250 mg/kg tenofovir disoproxil, and 166 mg/kg emtricitabine.

Flow cytometry and cell sorting. For surface staining, single cell suspensions prepared from peripheral blood, spleen, or mesenteric lymph nodes of humanized mice were stained with surface markers and analyzed on a CyAn ADP flow cytometer (Dako). For intracellular staining, cells were first stained with surface markers, and then fixed and permeabilized with cytofix/cytoperm buffer (BD Bioscience), followed by intracellular staining. FITC-conjugated anti-human HLA-DR(L243), IFN-γ (4S.B3), PE-conjugated anti-human CD38 (HIT2), CD303 (201A), PerCP/Cy5.5-conjugated anti-human CD4 (RPA-T4), PE/Cy7-conjugated anti-human CD8 (HIT8a), PB-conjugated anti-human CD14 (M5E2), IL-2 (MQ1-17H12), BV421- conjugated anti-human PD-1 (EH12.21H7), APC-conjugated anti-human CD11c (Bu15), and APC/Cy7-conjugated anti-human CD45 (HI30) were purchased from Biolegend. FITC-conjugated anti-HIV-1 p24 were purchased from Beckman Coulter. PE-conjugated anti-human active caspase3 (C92-605) was purchased from BD Pharmingen™. Pacific orange-conjugated anti-mouse CD45 (30-F11), PE/Texas red-conjugated anti-human CD3 (7D6), CD19 (SJ25-C1) and LIVE/DEAD Fixable Yellow Dead Cell Stain Kit were purchased from Invitrogen. Data were analyzed using Summit4.3 software (Dako).

For CD8 T cell sorting, after staining with viability dye and surface markers (anti-hCD45, mCD45, hCD3, hCD4, hCD8, hCD11c, hCD14, hCD123), CD8 T cells (hCD45^{±}mCD45⁻hCD3⁺hCD8⁺hCD4⁻) were sorted on a BD FACSAria II using a 70-mm nozzle and collected into Falcon™ round-bottom polypropylene tubes containing RPMI1640/10% FBS. The purity of sorted CD8 T cells was above 99%.

T cell stimulation and Intracellular cytokine staining. For non-specific stimulation, splenocytes from humanized mice were stimulated ex vivo with PMA (phorbol 12-myristate 13-acetate) (50 ng/ml) and ionomycin (1 uM) (Sigma, St Louis, Mo.) for 4 hours in the presence of brefeldin A (Biolegend). For antigen-specific stimulation, splenocytes from humanized mice were stimulated ex vivo with peptide pools (2 μg/ml for each peptide) for HIV-1 GAG protein (PepMix™ HIV (GAG) Ultra, JPT Innovation Peptide Solutions) for 3 hours without brefeldin A and then 5 hours in the presence of brefeldin A. Cells were then fixed and permeabilized with cytofix/cytoperm buffer (BD Bioscience), and intracellular staining was then performed.

HIV-1 genomic RNA detection in plasma. HIV-1 RNA was purified from the plasma with the QIAampkit® Viral RNA Mini Kit. The RNA was then reverse transcribed and quantitatively detected by real time PCR using the TaqMan® Fast Virus 1-Step PCR kit (ThermoFisher Scientific). The primers used for detecting the HIV Gag gene were (5′-GGTGCGAGAGCGTCAGTATTAAG-3′ (SEQ ID NO: 17) and 5′- AGCTCCCTGCTTGCCC ATA-3′ (SEQ ID NO: 18)). The probe (FAM-AAAATTCGGTTAAGGCCAGGGGGAAAGAA-QSY7 (SEQ ID NO: 19)) used for detection was ordered from Applied system and the reactions were set up following manufacturer's guidelines and were run on the QuantStudio 6 Flex PCR system (Applied Biosystems). The detection limit of the real-time PCR reaction is 4 copies per reaction. According, due to the relatively small volume of each bleeding in mice (around 50-100 μl total blood), the limit of detection of the assay is 400 copies/ml plasma. We arbitrarily set the copy numbers that are below detectable limit as 1.

Cell-associated HIV-1 DNA detection. To measure total cell-associated HIV-1 DNA, nucleic acid was extracted from spleen and bone marrow cells using the DNeasy mini kit (Qiagen). HIV-1 DNA was quantified by real time PCR. DNA from serial dilutions of ACH2 cells, which contain one copy of HIV genome in each cell, was used to generate a standard curve.

Cell-associated HIV-1 RNA detection. To measure total cell-associated HIV-1 RNA, nucleic acid was extracted from spleen or bone marrow cells using the RNeasy plus mini kit (Qiagen). HIV-1 RNA was detected as described above. The HIV-1 RNA expression levels were normalized to human CD4 mRNA (5′-GGGCTTCCTCCTCCAAGTCTT-3′ (SEQ ID NO: 20) and CCGCTTCGAGACCTTTGC (SEQ ID NO: 21)) controls and result was calculated as fold change in gene expression.

Viral outgrowth assay. Viral outgrowth assay was performed as reported (Laird et al., Methods Mol. Biol. 1354:239 (2016)). Serial dilutions of human cells from splenocytes of humanized mice (1×10⁶, 2×10⁵, 4×10⁴ human cells) were stimulated with PHA (2 μg/ml) and IL-2 (100 units/ml) for 24 hours. MOLT4/CCR5 cells were added on day 2 to enhance the survival of the cultured cells as well as to support and facilitate further HIV-1 replication. Culture medium containing IL-2 (NIH AIDS reagents program) and T cell growth factor (homemade as described in the standard protocol) was replaced on days 5 and 9. After 7 and 14 days of culture, supernatant from each well was harvested and HIV-1 RT-qPCR was performed to score viral outgrowth. Estimated frequencies of cells with replication-competent HIV-1 were calculated using limiting dilution analysis.

RNA-seq sequencing. Purified human CD8 T cells from spleens of humanized mice as described above were used to prepare RNA. The cDNA was prepared by using SMART Seq v4 Ultra Low RNA-Seq kit for 48 reactions kit (Clontech). bA Nextera kit was used for library construction, and sequencing was performed on Illumina HiSeq2500v4 with paired end sequencing for 50 cycles. Sequencing data fastq files for samples were processed in salmon workflow in a Linux server operating system to output gene-level abundance estimates and statistical inference as gene level raw counts. Those raw counts for samples were input into edgeR Program for differential gene expression analysis.

Statistics. Statistical analyses were performed using GraphPad Prism 5.0 software (GraphPad Software). Experiments were analyzed by two-tailed Student's t-test, or by one-way analysis of variance (ANOVA) and Bonferroni's post hoc test or Gehan-Breslow-Wilcoxon test, according to the assumptions of the test, as indicated in the figure legends for each experiment. *P<0.05, **P<0.01, ***P<0.001. All the data with error bars are presented as mean values±s.e.m. A P value less than 0.05 was considered significant.

Study approval. Human fetal liver and thymus tissues (gestational age of 16 to 20 weeks) were obtained from elective or medically indicated termination of pregnancy through a non-profit intermediary working with outpatient clinics (Advanced Bioscience Resources, Alameda, Calif.). Informed consent of the maternal donors is obtained in all cases, under regulation governing the clinic. The project was reviewed by the University's Office of Human Research Ethics, which has determined that this submission does not constitute human subjects research as defined under federal regulations [45 CFR 46.102 (d or f) and 21 CFR 56.102(c)(e)(1)]. All animal studies were approved by the University of North Carolina Institutional Animal Care and Use Committee.

### EXAMPLE 2

### cART Efficiently Suppresses HIV-1 Replication but Fails to Clear HIV-1 Reservoirs in Humanized Mice, Correlated with Low Levels of ISG Expression

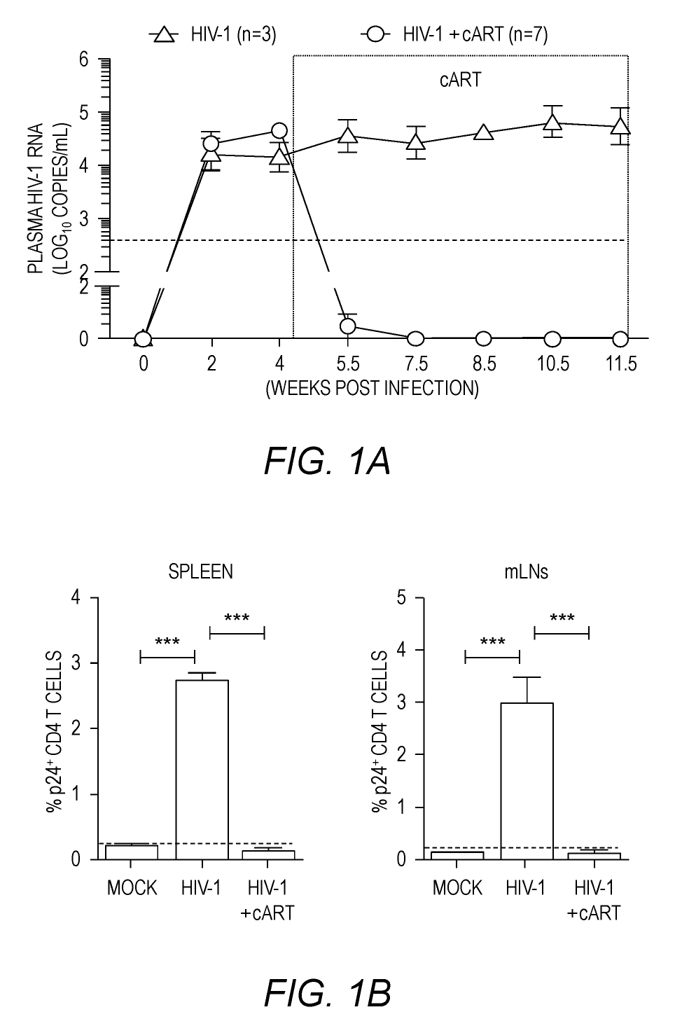

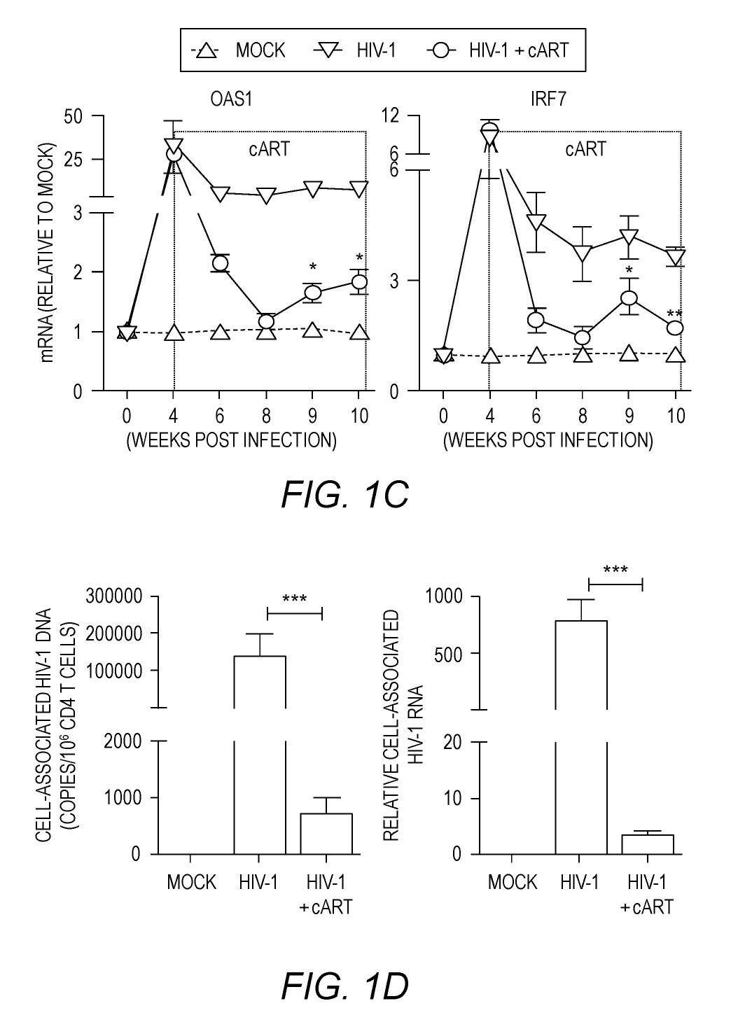

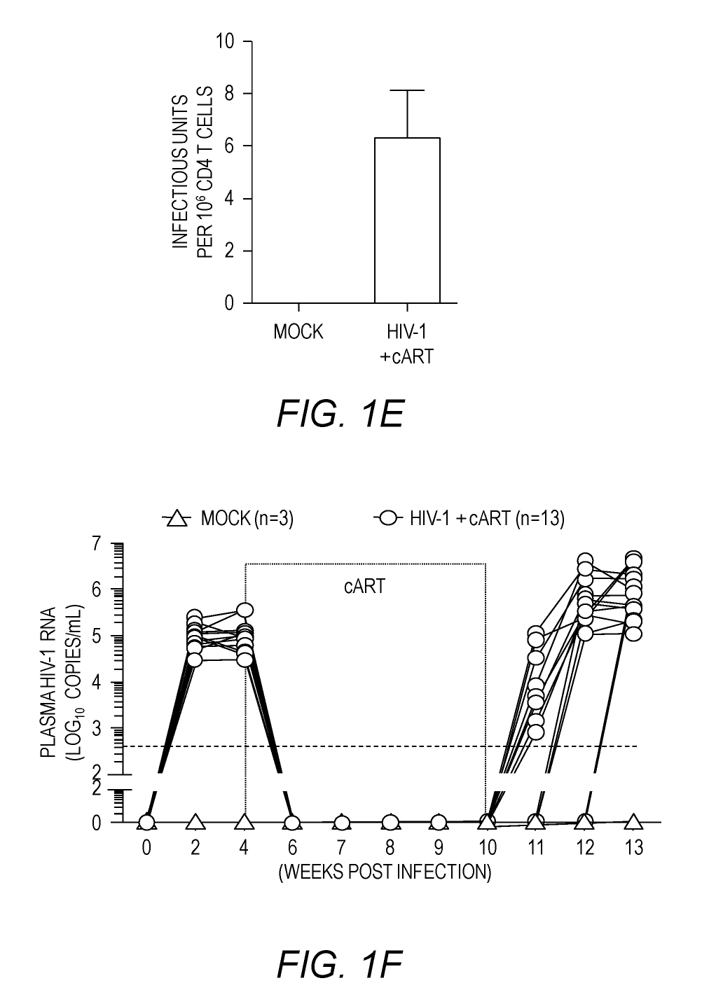

To functionally define the role of IFN-I in HIV-1 persistent infection and pathogenesis, humanized mice with a functional human immune system (hu-mice) were employed for modeling HIV-1 infection and immunopathogenesis (Shultz et al., Nat. Rev. Immunol. 12:786 (2012); Zhang et al., Cell. Mol. Immunol. 9:237 (2012)). It has been previously reported that persistent HIV-1 infection in hu-mice led to induction of IFN-I signaling, CD4 T cell depletion, aberrant immune activation and expression of exhaustion marker PD-1 on T cells (Zhang et al., Cell. Mol. Immunol. 9:237 (2012); Li et al., GPLoS Patholog. 10:e1004291 (2014); Seung et al., PLoS One 8:e77780 (2013)). As in human patients, cART can efficiently inhibit HIV-1 replication in hu-mice (Halper-Stromberg et al., Cell 158:989 (2014); Choudhary et al., J. Virol. 86:114 (2012)). It was found here that plasma viremia decreased to undetectable levels (<400 genome copies/ml) in all HIV-infected hu-mice within 3 weeks after cART treatment (FIG. 1A). HIV-1 replication in lymphoid organs was also effectively inhibited by cART (FIG. 1B). However, as observed in some patients (Fernandez et al., J. Infect. Dis. 204:1927 (2011); Dunham et al., J. Acquir. Immune Defic. Syndr. 65:133 (2014)), cART failed to completely reduce ISGs expression in HIV-1 infected mice to the level of uninfected hu-mice (FIG. 1C). HIV-1 reservoirs, measured by cell-associated HIV-1 DNA and RNA (FIG. 1D), and cells with infectious HIV-1 (FIG. 1E), were still detectable in lymphoid organs of cART-treated hu-mice. Similar to cART-treated patients, HIV-1 reservoirs persist stably and virus rebounds rapidly after cART cessation (FIG. 1F).

### EXAMPLE 3

### IFNaR Blockade during cART-Suppressed HIV-1 Infection Reverses Aberrant Immune Activation

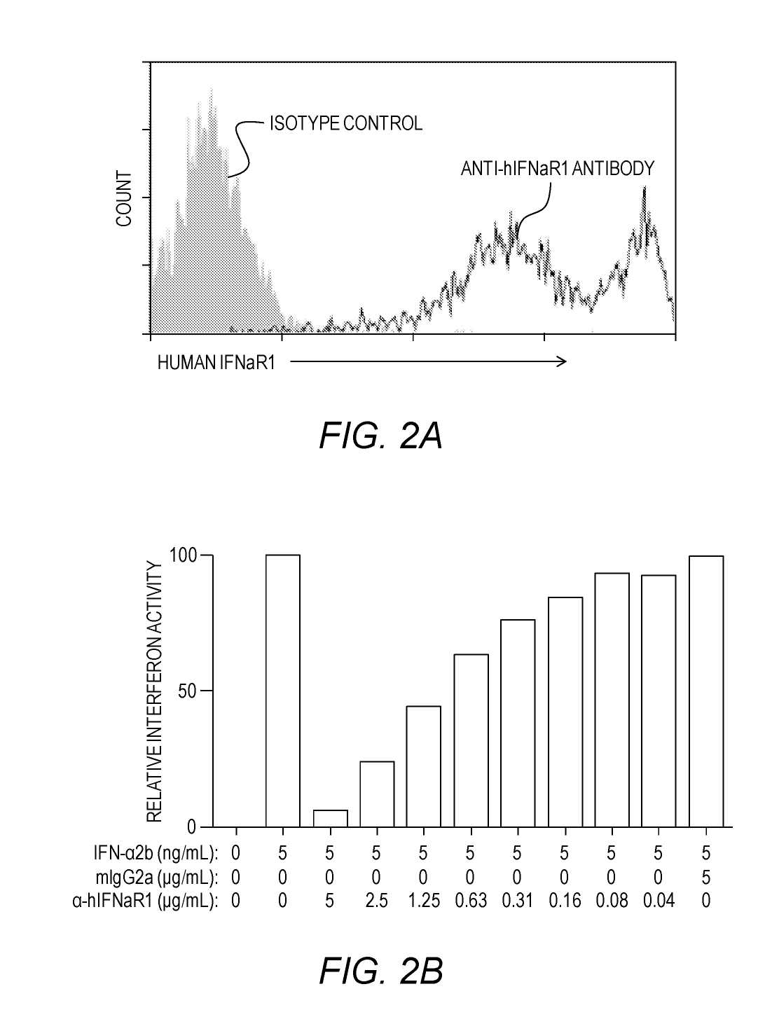

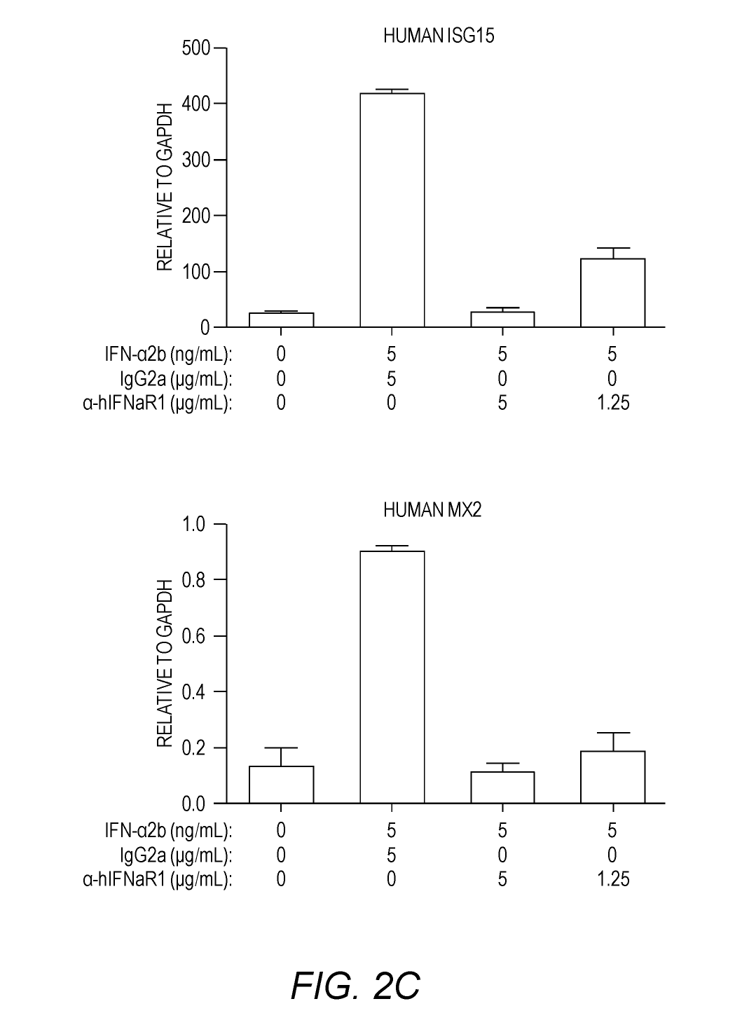

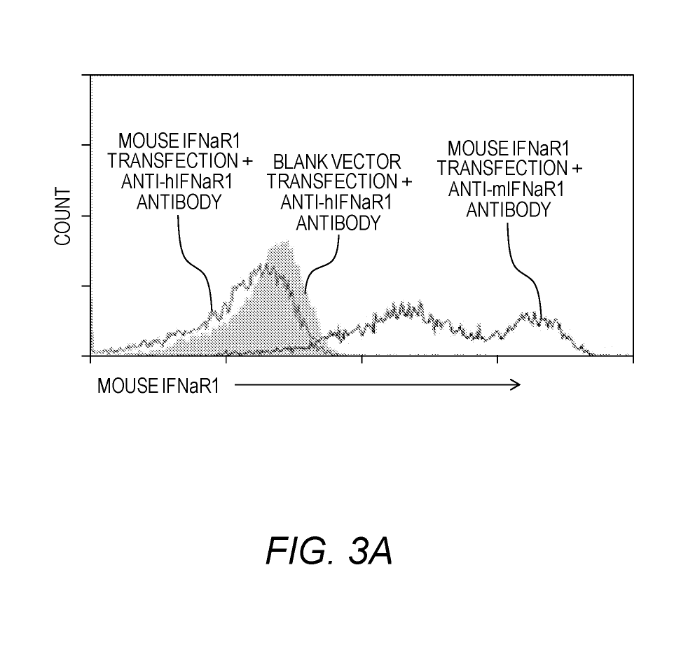

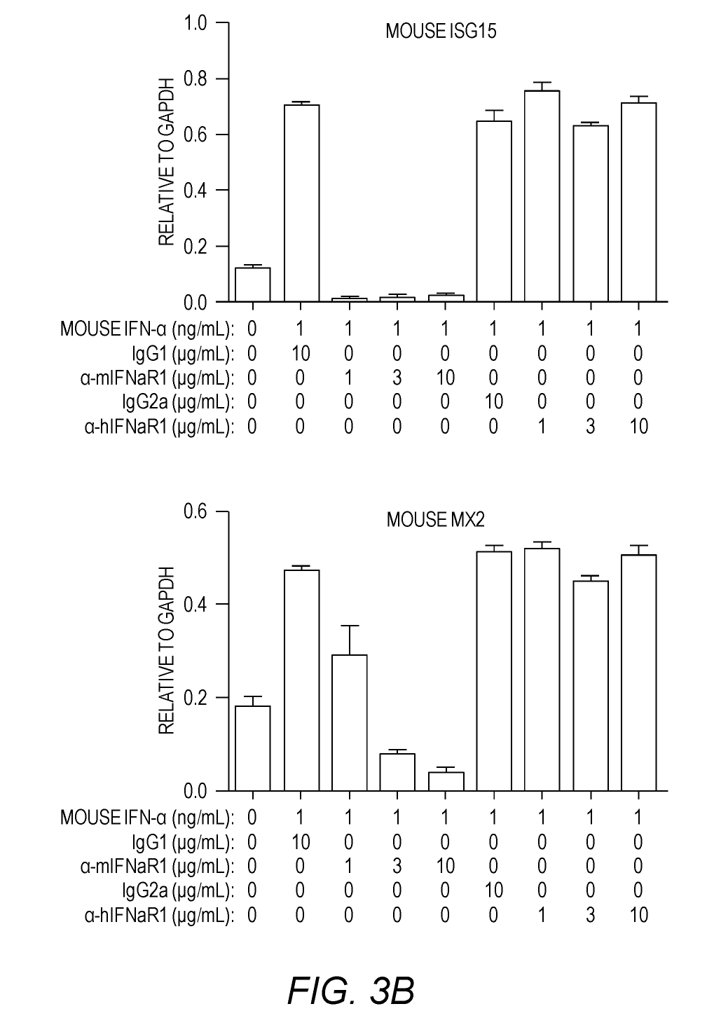

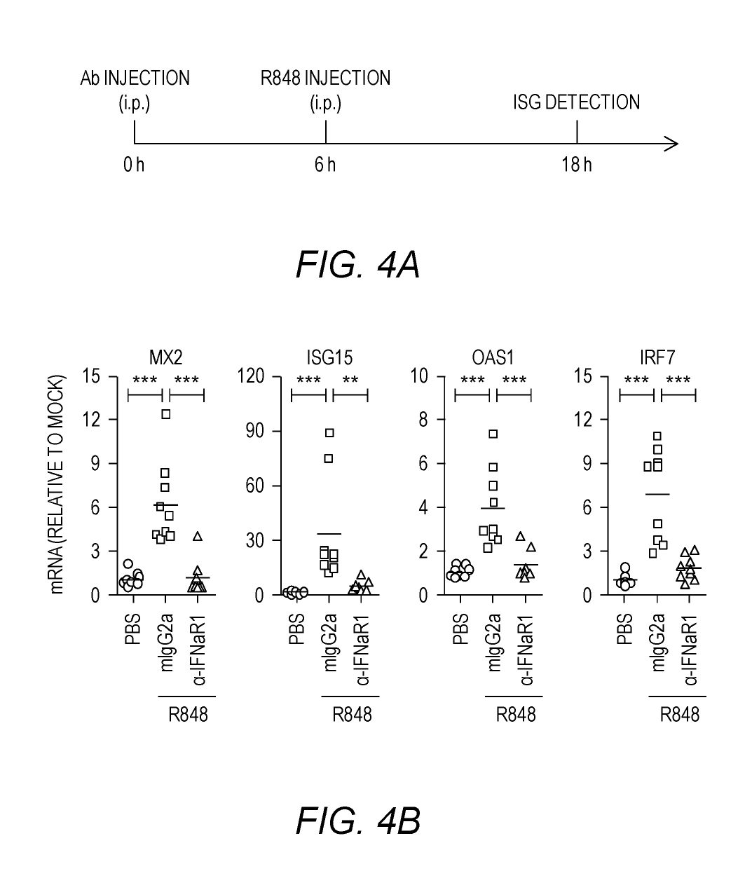

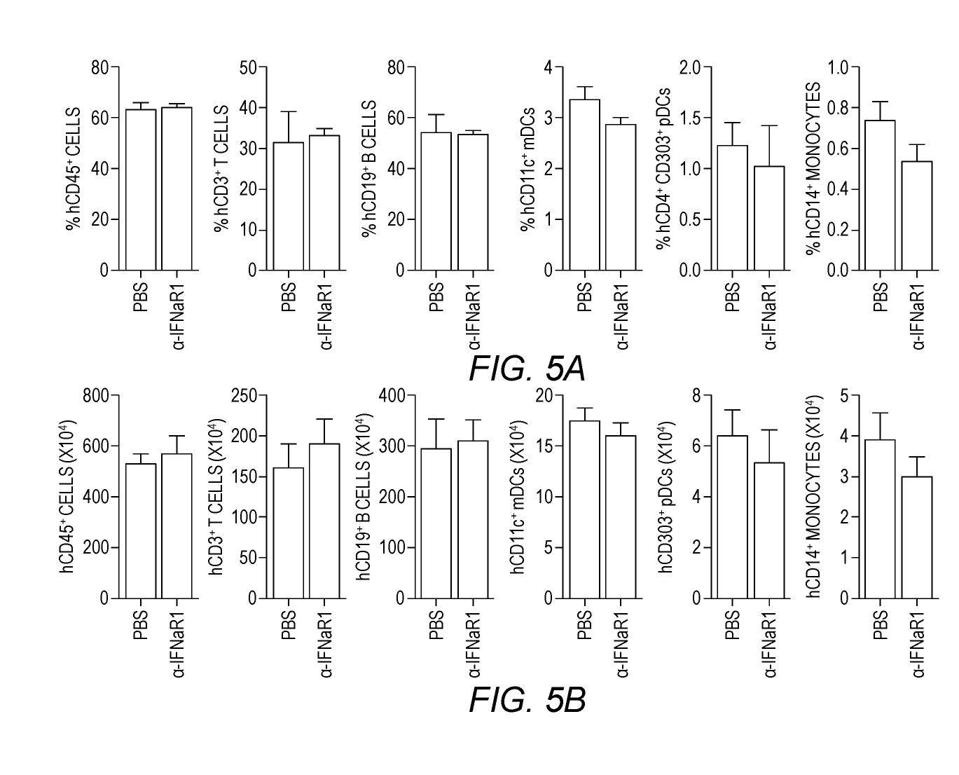

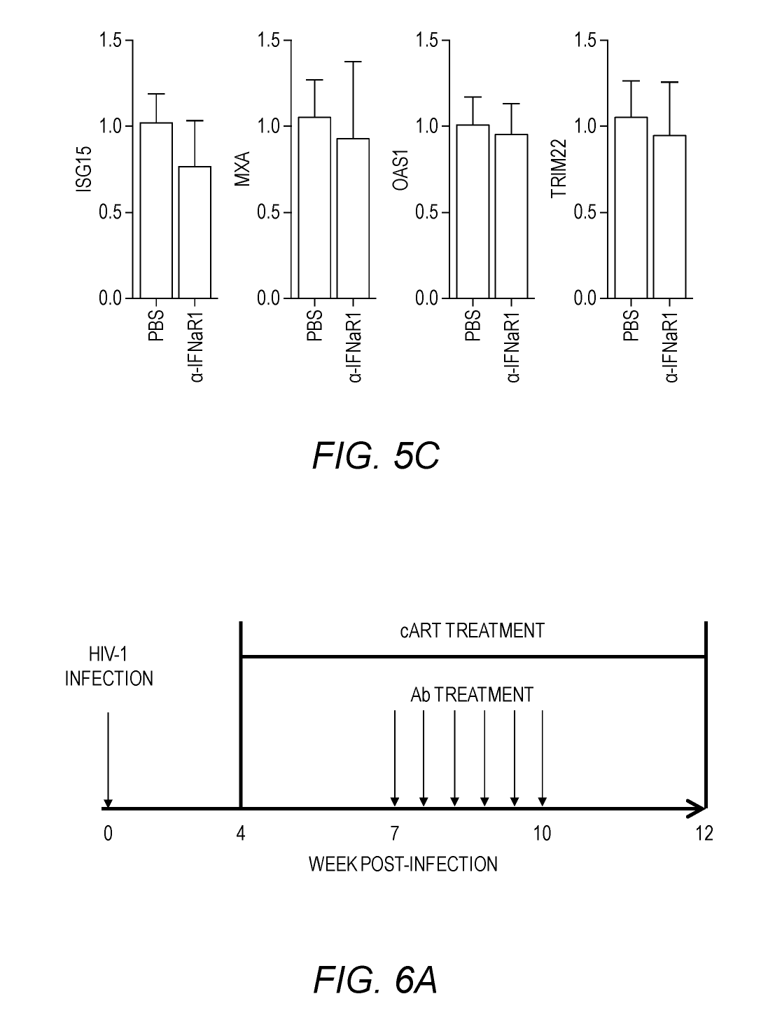

It is reported that abnormally elevated levels of IFN-I signaling and ISG expression persist in some patients even under extensive cART (Fernandez et al., J. Infect. Dis. 204:1927 (2011); Dunham et al., J. Acquir. Immune Defic. Syndr. 65:133 (2014)), which may impede immune recovery and foster viral persistence (Bosinger et al., Curr. HIV/AIDS Rep. 12:41 (2015); Deeks, Annu Rev Med 62:141 (2011)). It was hypothesized that IFNaR blockade in the presence of cART would reverse hyper-immune or inflammatory activation and facilitate recovery of functional anti-HIV-1 adaptive immune responses, thereby enabling control of cART-resistant HIV-1 reservoirs. To block IFN-I signaling in hu-mice, a monoclonal antibody to human IFN-α/β receptor 1 (α-IFNaR1) was developed, that specifically binds to human IFNaR1 (FIG. 2A) and inhibits human IFN-1 activity (FIG. 2B-2C). The α-IFNaR1 mAb didn't bind to mouse IFNaR1 (FIG. 3A) nor block mouse IFN-I activity in mouse cells (FIG. 3B). Furthermore, it was shown that the α-IFNaR1 mAb effectively blocked ISG induction in vivo in response to the Toll-like receptor 7/8 agonist R848 (Resiquimod) in hu-mice (FIGS. 4A-4B). Treatment with α-IFNaR1 mAb alone affected neither the percentage nor the number of human leukocyte subsets (FIG. 5A-5B), and administration of α-IFNaR1 did not affect the expression of ISGs in splenocytes in humanized mice (FIG. 5C).

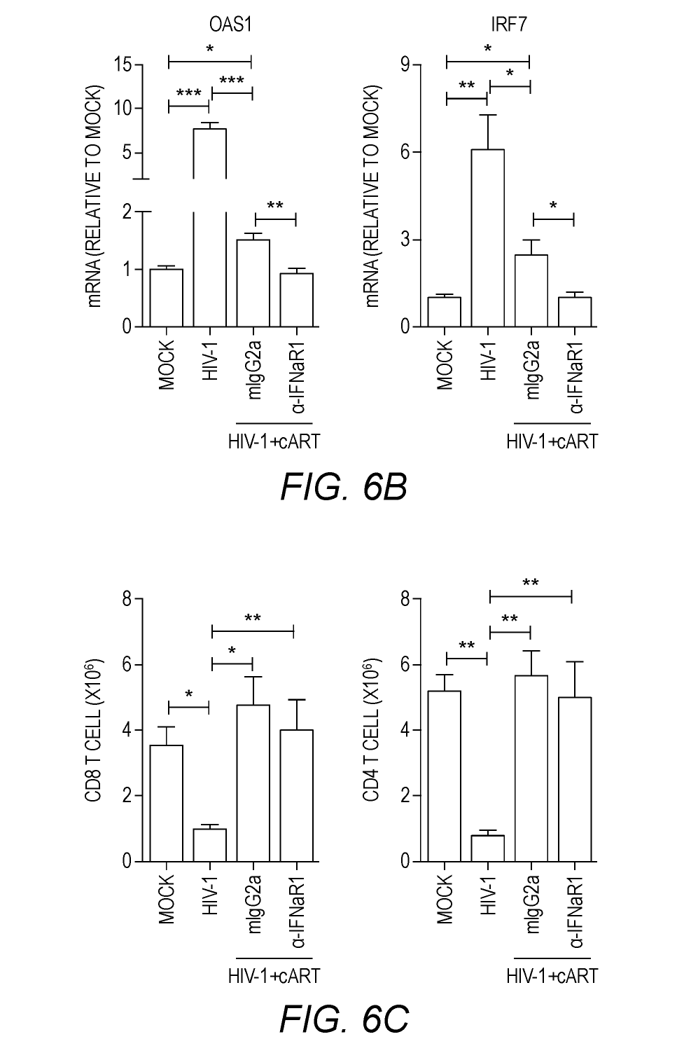

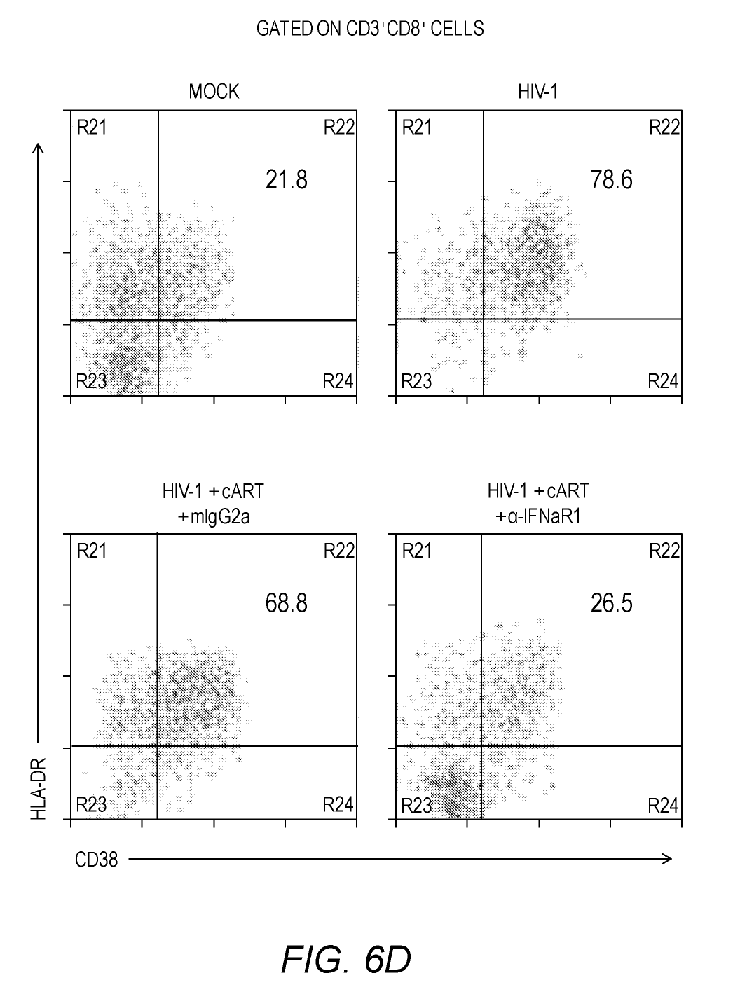

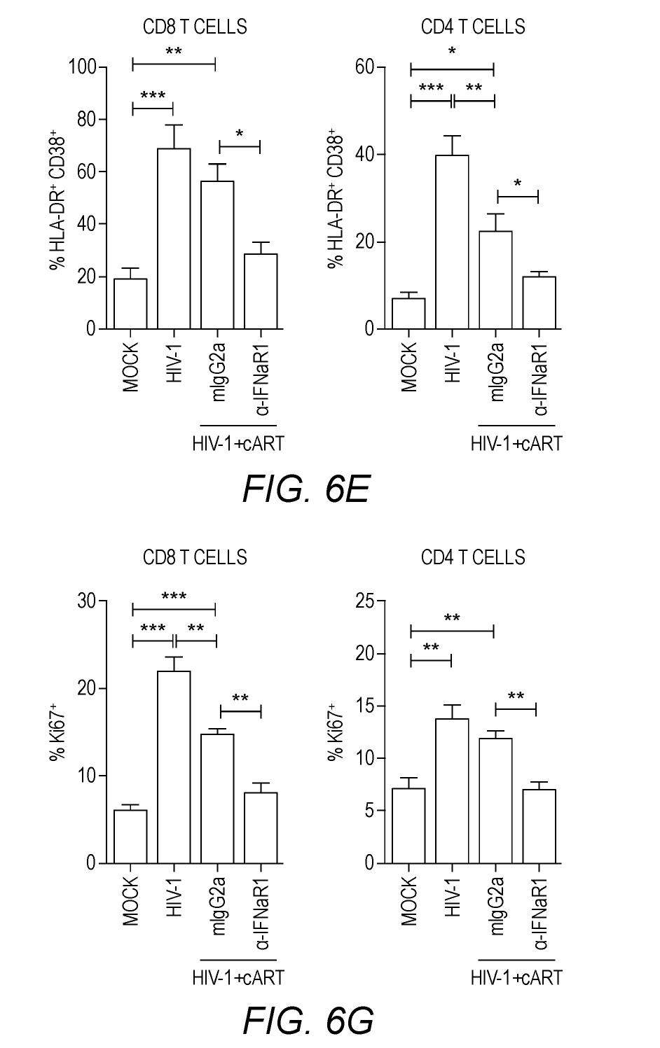

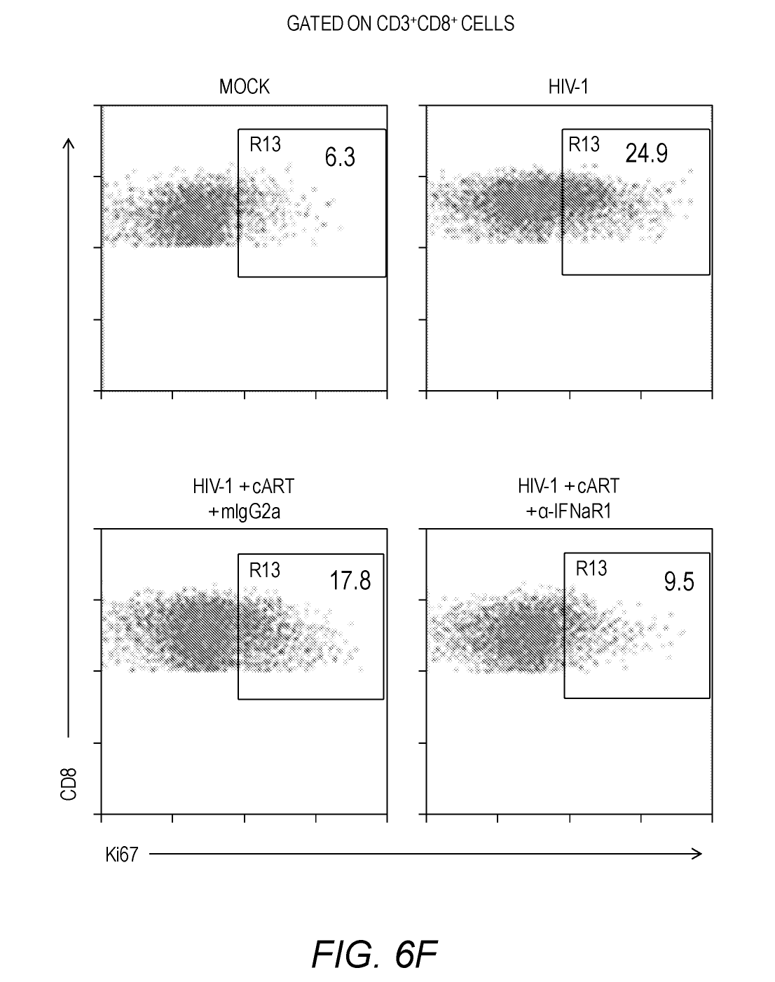

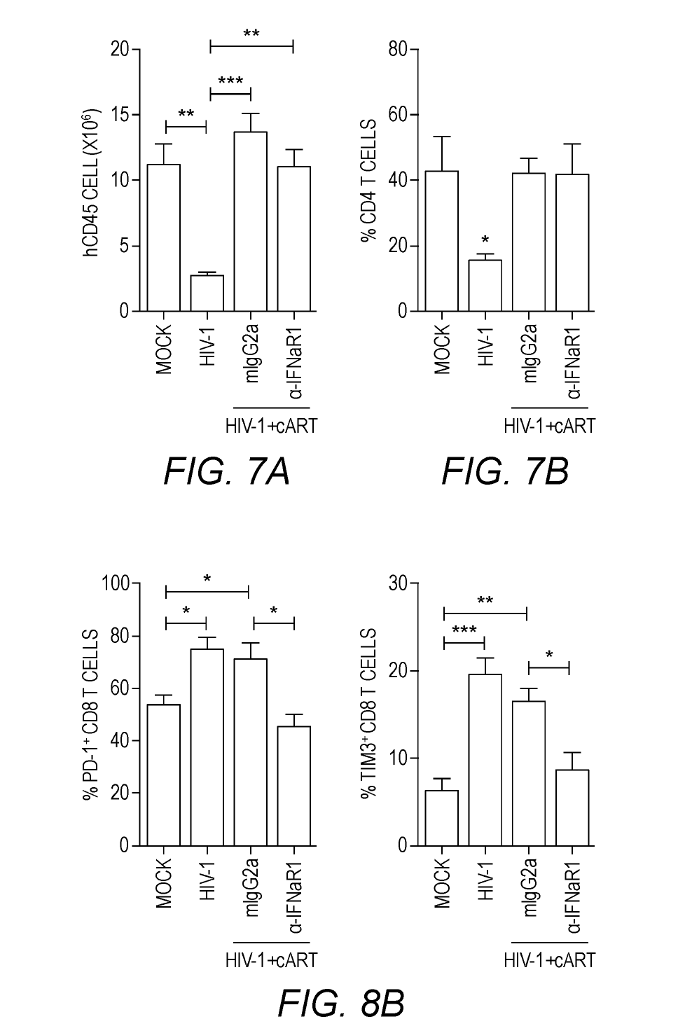

Next, the effect of IFNaR blockade on T-cell activation and functions in the presence of cART treatment was analyzed in HIV-1 infected hu-mice. HIV-1-infected hu-mice that were fully cART-suppressed were treated with α-IFNaR1 mAb for 3 weeks (from 7 to10 wpi, FIG. 6A). As in some cART-treated human patients (23, 24), cART failed to completely suppress expression of ISGs (FIG. 1C and FIG. 6B). In contrast, IFNaR blockade efficiently suppressed HIV-induced ISG expression in cART-treated hu-mice (FIG. 6B and FIG. 5A). HIV-1 persistent infection in hu-mice also induced CD8 and CD4 T cell hyper-immune activation and proliferation as indicated by the expression of activation marker CD38/HLA-DR and proliferation marker Ki67. Although cART alone significantly rescued the number of human T cells and total human leukocytes (FIG. 6C and FIGS. 7A-7B), it only slightly decreased the expression level of CD38/HLA-DR (FIGS. 6D-6E) and Ki67 on T cells (FIGS. 6F-6G). Both CD8 and CD4 T cells from cART-treated hu-mice still expressed significantly higher levels of activation (FIGS. 6D-6E) and proliferation (FIGS. 6F-6G) markers as compared to uninfected hu-mice. Interestingly, IFNaR blockade significantly reversed aberrant CD8 T-cell activation and proliferation in the presence of cART (FIGS. 6D-6G).

### EXAMPLE 4

### IFNaR Blockade Reverses the Exhaustion Phenotype of Human T Cells and Restores Anti-HIV-1 T Cell Function

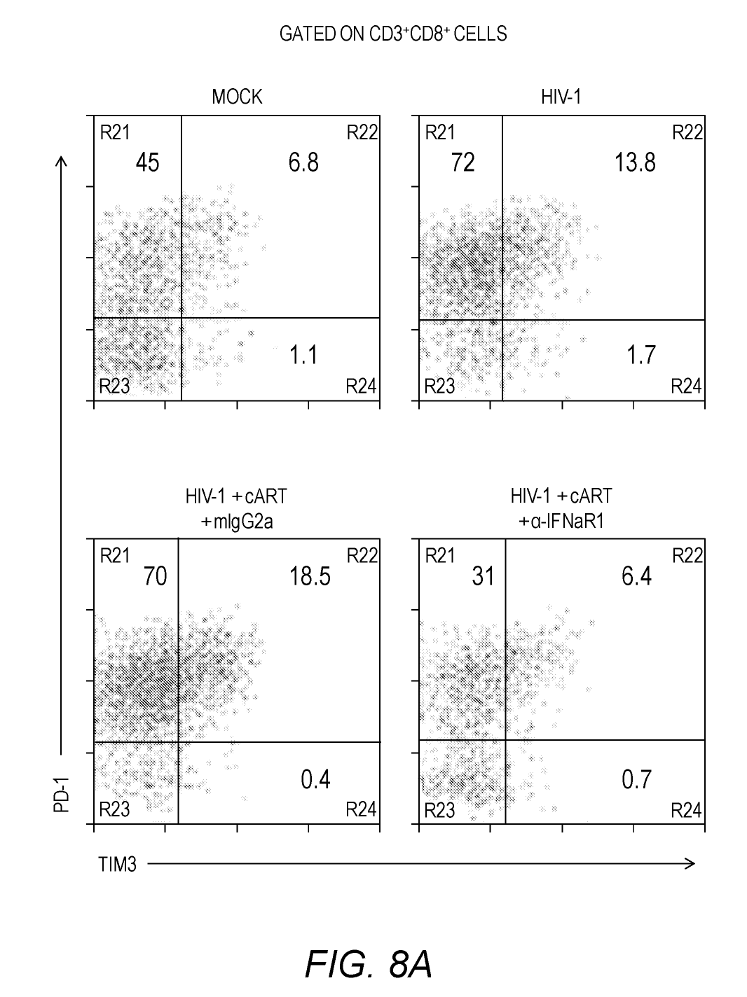

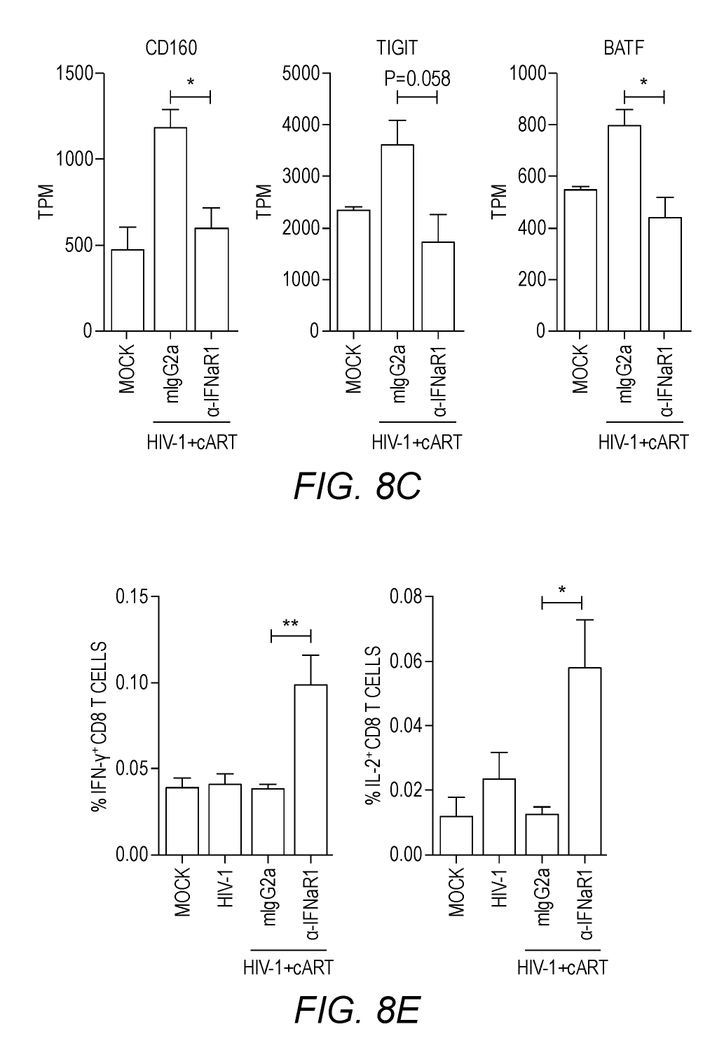

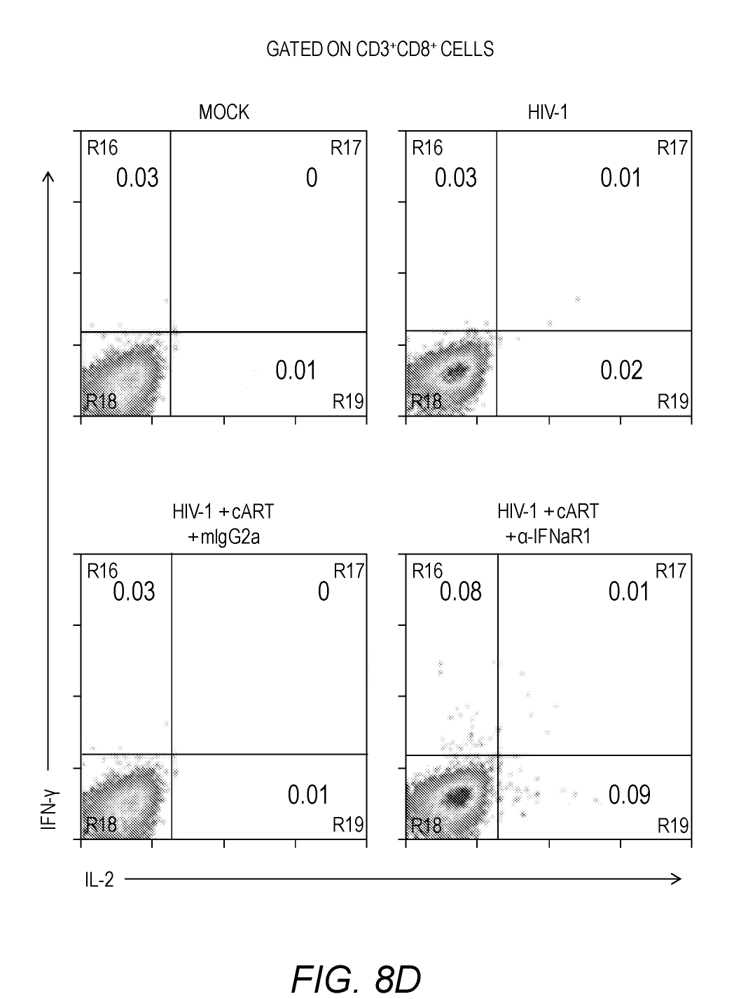

Despite successful viral inhibition by cART, T cells from patients with poor immune reconstitution sustained higher PD-1 expression (Grabmeier-Pfistershammer et al., J. Acquir. Immune Defic. Syndr. 56:118 (2011)). It was therefore investigated whether IFNaR blockade could reverse PD-1 and other exhaustion markers expression and rescue HIV-1 specific T cell function in the presence of cART. HIV-1 persistent infection in hu-mice induced both PD-1 and TIM-3 expression on CD8 T cells (FIGS. 8A-8B). It was found that cART alone failed to significantly reduce the expression of PD-1 and TIM-3 on CD8 T cells (FIGS. 8A-8B). Interestingly, IFNaR blockade combined with cART completely reduced expression of PD-1 and TIM-3 on CD8 T cells (FIGS. 8A-8B). Whole transcriptome sequencing of purified human CD8 T cells revealed that cART plus IFNaR blockade also significantly reduced the expression of other T-cell exhaustion markers including CD160, TIGIT (T-Cell Immunoreceptor with Ig and ITIM domains) and BATF (basic leucine transcription factor, ATF-like) (FIG. 8C) (Wherry et al., Nat. Rev. lmmunol. 15:486 (2015)).

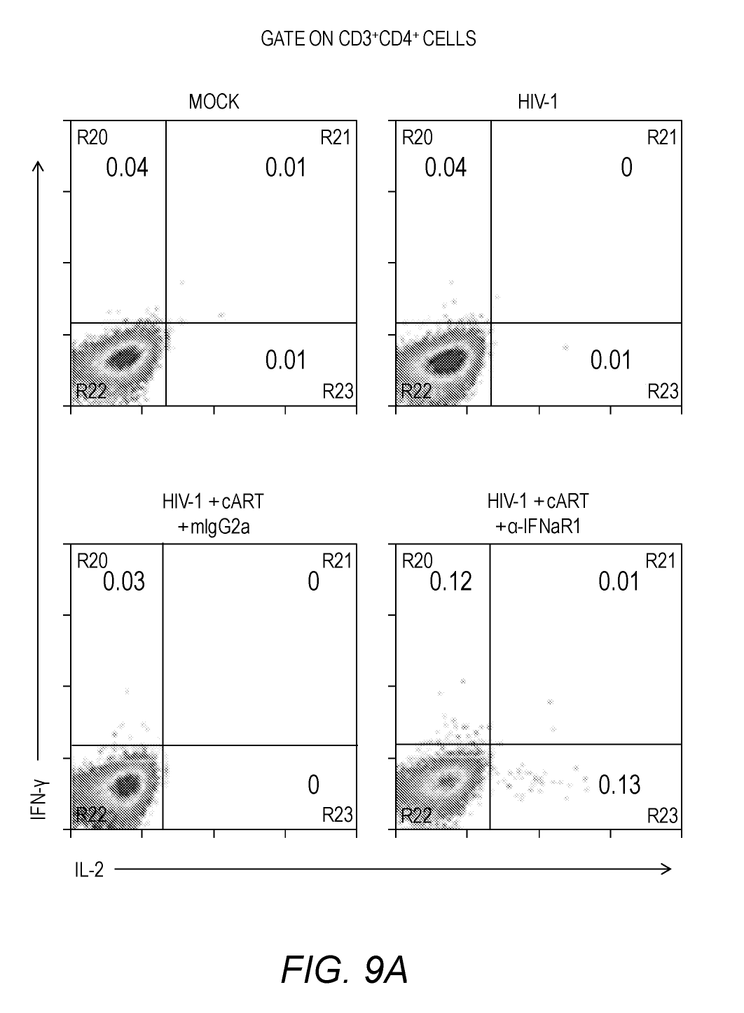

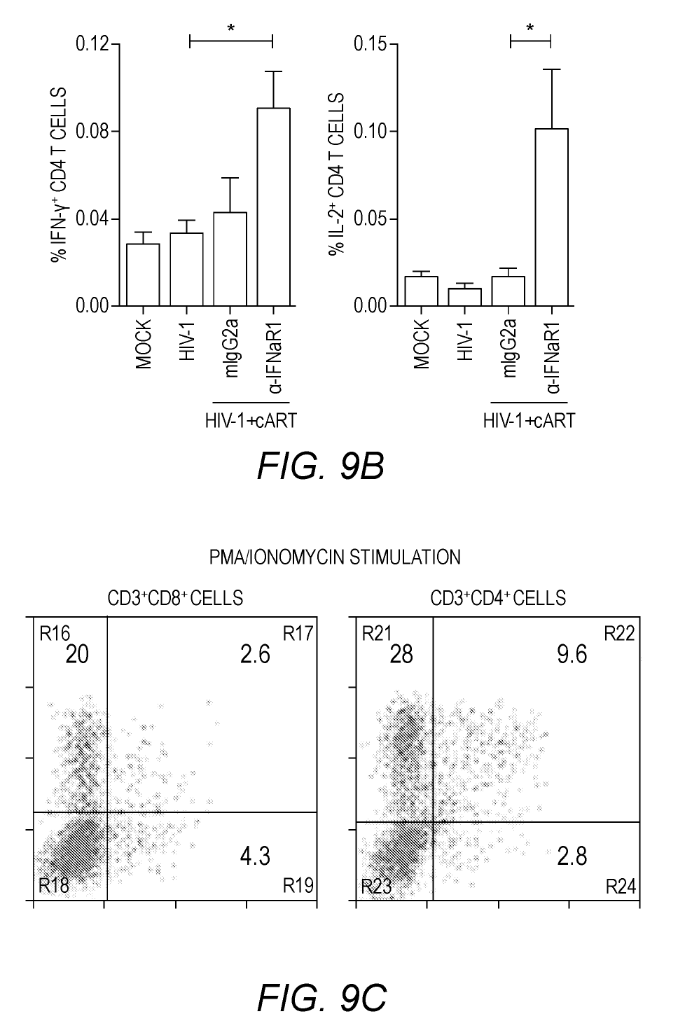

The function of HIV-1-specific T cells after IFNaR blockade was further determined. When stimulated with HIV-1 Gag peptide pools ex vivo, both CD8 and CD4 T cells from hu-mice with cART and IFNaR blockade (but not cART alone) produced significantly higher levels of IFN-γ and IL-2 (FIGS. 8D-8E and FIGS. 9A-9C), indicating that IFNaR blockade also rescued the function of HIV-1 specific T cell responses. Taken together, these results indicate that, in the presence of cART, IFNaR blockade can reverse aberrant immune activation, T cell exhaustion, and rescue anti-HIV-1 immune responses.

### EXAMPLE 5

### IFNaR Blockade During cART Reduces cART-Resistant HIV-1 Reservoirs

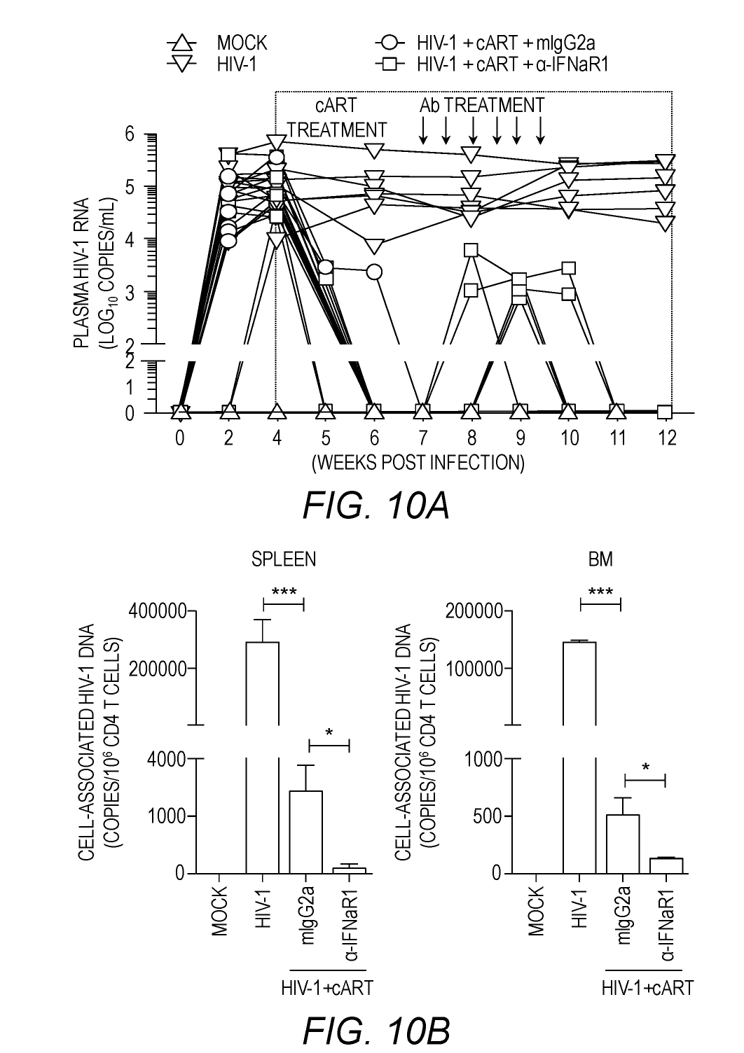

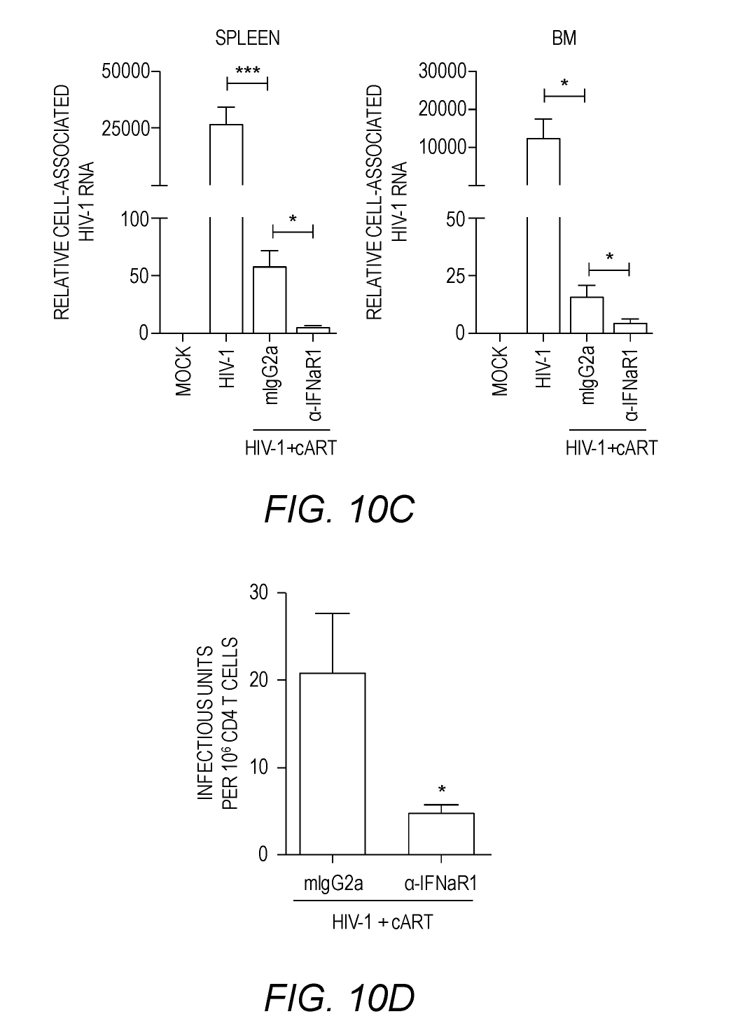

The administration of cART cannot achieve HIV-1 eradication and virus rebounds quickly after cART discontinuation owing to the persistence of HIV-1 reservoirs during cART (Katlama et al., Lancet 381:2109 (2013); Archin et al., Nat. Rev. Microbial. 12:750 (2014)). It was demonstrated that adaptive immune response contributes to the control of cART resistant reservoirs (Deng et al., Nature 517:381 (2015); Shan et al., Immunity 36:491 (2012)). Thus the improvement of anti-HIV-1 adaptive immune response by therapeutic vaccine or by other immune modulators has been proposed as immunological strategies for HIV cure (Barouch et al., Science 345:169 (2014); Siliciano, Nat. Med. 20:480 (2014)). It was postulated that the reversal of immune hyper-activation and the induction of elevated anti-HIV-1 T-cell response by blocking IFNaR might reduce the size of the cART-resistant HIV-1 reservoir and control HIV-1 rebound post-cART cessation. HIV-1-infected mice that were fully cART-suppressed were treated with α-IFNaR1 mAb for 3 weeks during 7-10 wpi (FIG. 10A). Interestingly, IFNaR blockade led to low blips of HIV-1 replication, which returned to undetectable levels after stopping α-IFNaR1 mAb treatment, in the presence of cART (FIG. 10A). Thus, IFNaR blockade induced activation of HIV-1 reservoirs. It was speculated that the increased anti-HIV-1 T cell immune responses by IFNaR blockade would eliminate or control the HIV-1 expressing cells and finally reduce HIV-1 reservoirs. Next, the HIV-1 reservoir size was analyzed in lymphoid organs 2 weeks after IFNaR blockade. Cell-associated HIV-1 DNA and RNA were measured by PCR, and replication-competent HIV-1 by the quantitative virus outgrowth assay. It was found that IFNaR blockade reduced cell-associated HIV-1 DNA by 14-fold in the spleen and by 4.4-fold in the bone marrow (FIG. 10B). Cell-associated HIV-1 RNA was also reduced in both spleen (17.7 folds) and bone marrow (4.4 folds) (FIG. 10C). More importantly and consistently, IFNaR blockade significantly reduced the size of replication-competent HIV-1 reservoirs measured by quantitative virus outgrowth assay (FIG. 10D).

### EXAMPLE 6

### IFNaR Blockade During cART Delays HIV-1 Rebound Post-cART Cessation

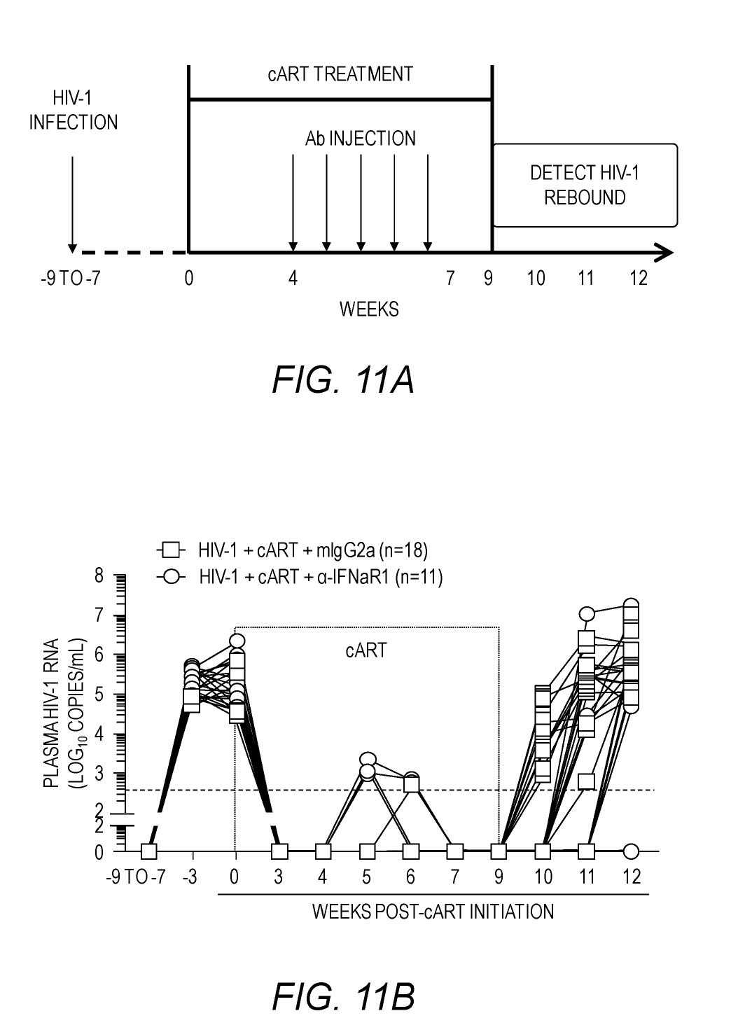

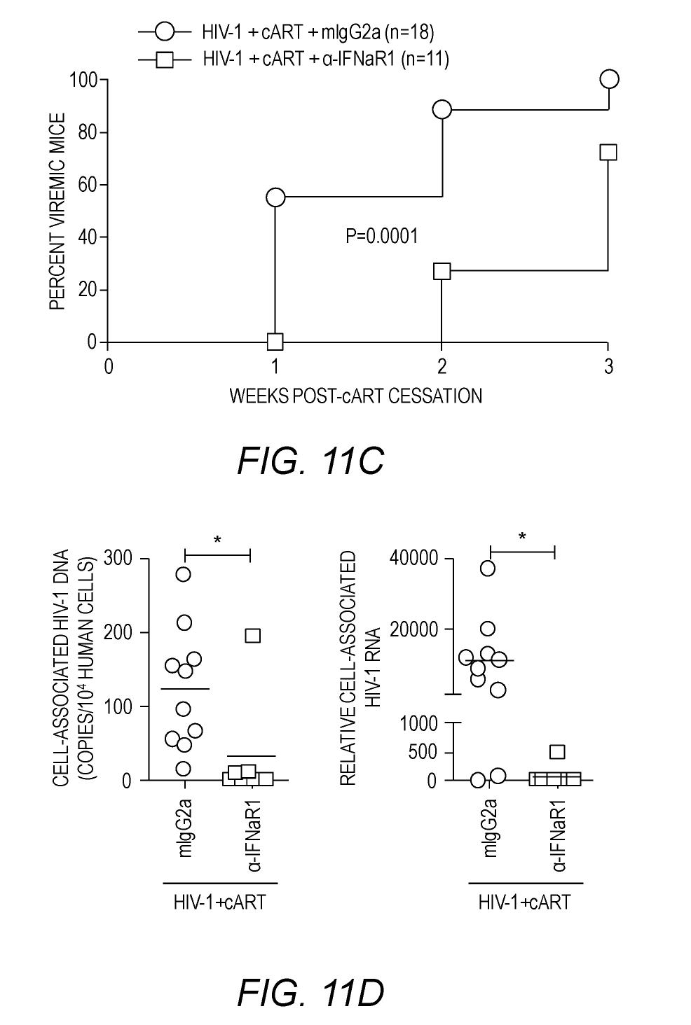

Finally, the effect of IFNaR blockade on HIV-1 rebound post-cART discontinuation was analyzed. Humanized mice with persistent HIV-1 infection received cART, followed with 5 injections of α-IFNaR1 mAb after full suppression of HIV-1 (FIG. 11A). cART was stopped 2.5 weeks after the last α-IFNaR1 mAb injection and virus rebound monitored after cART discontinuation. One week post-cART cessation, HIV-1 rebounded in 56% (10/18) of the control-treated hu-mice, but in none of the hu-mice treated with IFNaR1 blockade (FIGS. 11B-11C and Table 1). By the second week, 89% (16/18) of the control-treated hu-mice became HIV-1 positive while only 27% (3/11) of α-IFNaR1 treated hu-mice showed detectable viremia in the blood (FIGS. 11B-11C and Table 1). It was also found that cell associated HIV-1 DNA and RNA in PBMCs was significantly lower in the α-IFNaR1 treated group comparing control antibody treated group (FIG. 11D). By the third week, HIV-1 rebounded in all hu-mice (18/18) in the cART-only group and rebounded in 73% (8/11) of the cART plus IFNaR blockade hu-mice (FIGS. 11B-11C and Table 1). The viral load in those hu-mice with rebounded HIV-1 at the third week post-cART cessation reached the same level between the two treatment groups. Taken together, it was concluded that IFNaR blockade reduced cART-resistant HIV-1 reservoirs and significantly controlled HIV-1 rebound post cART cessation.

The contribution of IFN-I signaling to HIV-1 persistence during chronic infection is not clearly defined (Doyle et al., Nat. Rev. Microbiol. 13:403 (2015); Bosinger et al., Curr. HIV/AIDS Rep. 12:41 (2015)). Using humanized mice that support HIV-1 persistent infection, it was shown here that cART efficiently inhibited HIV-1 replication and rescued T cell number in hu-mice with persistent HIV-1 infection. However, low levels of ISG expression, aberrant immune activation, T cell exhaustion and HIV-1 reservoir persisted stably in cART-treated hu-mice. It was demonstrated here that IFNaR blockade in cART-treated hu-mice reversed T-cell hyper-immune activation, rescued anti-HIV-1 T cell immunity and reduced the size of HIV-1 reservoirs.

Despite efficient suppression of HIV-1 replication with cART, abnormally elevated IFN-I signaling persists in some individuals, which may impede immune recovery and foster viral persistence (Fernandez et al., J. Infect. Dis. 204:1927 (2011); Dunham et al., J. Acquir. Immune Defic. Syndr. 65:133 (2014); Deeks, Annu Rev Med 62:141 (2011)). It was found here that in hu-mice persistently infected with HIV-1, cART efficiently inhibited viral replication, but failed to reverse elevated ISG expression, T cell immune activation and exhaustion. This model thus partially recapitulates the phenotype of those immune-non-responder patients. It was shown that blocking IFN-I signaling by using the newly developed anti-IFNaR1 mAb completely inhibited ISG expression, reversed T cell immune activation, exhaustion and rescued T cell function. These results agree with recent findings showing that persistent IFN-I signaling plays a detrimental role during chronic lymphocytic choriomeningitis virus (LCMV) infection and blocking IFN-I signaling by IFNaR antibody could reverse T cell exhaustion and enhance antiviral immune response (Wilson et al., Science 340:202 (2013); Teijaro et al., Science 340:207 (2013)). The IFNaR blocking antibody will thus facilitate novel therapeutic development aimed at those “difficult-to-treat HIV-1-infected patients” with sustained IFN-I signaling during cART (Fernandez et al., J Infect. Dis. 204:1927 (2011); Dunham et al., J. Acquir. Immune Defic. Syndr. 65:133 (2014); Zhang et al., AIDS 27:1283 (2013)).

HIV-1 reservoirs are refractory to antiretroviral therapies (ART) and remain the major barrier to curing HIV-1 (Katlama et al., Lancet 381:2109 (2013); Archin et al., Nat. Rev. Microbiol. 12:750 (2014)). It is reported here that IFNaR blockade transiently increased HIV-1 RNA in the blood (viral load “blipping”) during cART, indicating that IFN-I signaling contributed to the low replication or latency of the HIV-1 reservoirs. Multiple mechanisms may lead to the reduction of HIV-1 reservoir size after IFNaR blockade during cART. The rescued immune response could target the HIV-1 reservoirs with elevated gene expression and kill the reservoir cells. Other factors, including HIV-1 induced death of reservoir cells, reduced general T cell activation and proliferation after IFNaR blockade may also contribute to the reduction of HIV-1 reservoir size. The underlying mechanism of reservoir reduction by IFNaR blockade will be further elucidated in the future. Therefore, blocking IFN-I signaling in cART-treated subjects may provide a novel therapeutic approach for HIV-1 cure (Barouch et al., Science 345:169 (2014)).

In a recent report, blocking IFN-I signaling with an antagonistic IFNα2 mutant (IFN-ant, with increased IFNaR2 binding but diminished IFNaR1 binding activity (Levin et al., Sci. Signal 7:ra50 (2014))) during acute phase (0-4 weeks post infection) of SIV infection in rhesus monkeys leads to elevated SIV replication and accelerated disease progression (Sandler et al., Nature 511:601 (2014)). Conversely, while pre-infection IFN-I administration results in decreased SIV transmission, continued IFN-α2a treatment appears to induce IFN-I desensitization and decrease antiviral gene expression, resulting in increased SIV replication and accelerated CD4 T-cell loss (Sandler et al., Nature 511:601 (2014)). This study has major differences from the present studies in that IFN-I signaling is blocked (or desensitized) only during acute SIV infection. The higher levels of SIV infection probably lead to the accelerated disease progression during late stage of infection, in the absence of IFN-I blocking. It is generally believed that persistent IFN-signaling during chronic infection can lead to general immune suppression (Crouse et al., Nat. Rev. Immunol. 15:231 (2015)). Therefore, IFN-I signaling is beneficial during acute stage to inhibit or prevent virus infection but becomes harmful during chronic stage of HIV-1 infection. Blocking IFN-I signaling with either the IFNaR mAb or the antagonistic IFNα2 mutant protein in rhesus monkeys with persistent SIV infection and cART will be of great interest to further clarify these therapeutic strategies.

Several recent reports have shown that the administration of IFN-α in HIV-1 mono-infected or HIV-1/HCV co-infected patients leads to reduction of cell associated viral RNA and DNA in the blood cells of a subset of treated patients (Azzoni et al., J. Infect. Dis. 207:213 (2013); Sun et al., J. Infect. Dis. 209:1315 (2014); Jiao et at, Antiviral Res. 118:118 (2015); Moron-Lopez et al., J. Infect. Dis. 213:1008 (2016)). The study by Livio Azzoni et al. reports that long-term administration of IFN-α during and after ART in HIV-1 infected patients leads to suppression of HIV-1 rebound in ˜40% of patients, whose PBMC-associated HIV-1 DNA (after 12 weeks with IFNa only but no ART) is lower when compared with their PBMCs during ART alone when normalized to their CD4 T cell counts. However, HIV-1 reservoirs (cell-associated DNA) were not significantly changed by IFN-I treatment during ART. The administration of IFN-I may induce the migration of activated CD4 T cells into lymphoid organs and subsequently reduction in the peripheral blood (Massanella et al., Antivir. Ther. 15:333 (2010)), thus the reduction may be due to the redistribution of HIV-1 reservoir cells to lymphoid organs induced by IFN-α. Interestingly, treatment of HIV-1/HCV co-infected patients with INFα/Ribavirin appears to lead to a significant reduction of both CD4 and CD8 T cells(Azzoni et al., J, Infect. Dis. 207:213 (2013); Jiao et al., Antiviral Res. 118:118 (2015); Moron-Lopez et al., J. Infect. Dis. 213:1008 (2016)), which is consistent with our finding that IFN-I during chronic HIV-1 infection contributes to T cell depletion. In addition, low level HIV-1 replication in the presence of cART may also contribute to the HIV-1 reservoir pool (Lorenzo-Redondo et al., Nature 530:51 (2016)). High levels of IFN-I may inhibit the low level HIV-1 replication as well as enhance anti-HIV immune responses (Tomescu et al., Aids 29:1767 (2015)). Therefore, IFN-I signaling may play complex roles during acute and chronic phases of HIV-1 infection, both inhibiting viral replication and fostering viral persistence by inducing immune dysfunction.

### EXAMPLE 7

### Methods

Construction of humanized mice: NRG (NOD-Rag2^{-/-}γc^{-/-}) and NSG-A2 (NOD.Cg-Prkdc^{scid}γc^{-/-}Tg(HLA-A2.1) mice were obtained from the Jackson Laboratory. Humanized NRG/NSG-A2 mice with a functional human immune system were generated by intrahepatic injection of new born mice with human fetal liver derived CD34⁺ hematopoietic progenitor cells as previously reported (Li et al., PLoS Pathogens 10(7):e1004291 (2014)). Humanized BLT (bone marrow/liver/thymus) mice were generated as previously reported(Namikawa et al., Science 242:1684 (1988)). Briefly, 6 to 8 weeks old NRG mice were sub-lethally irradiated and anesthetized, and ˜1-mm³ fragments of human fetal thymus were implanted under the kidney capsule. CD34⁺ hematopoietic progenitor cells purified from fetal liver of the same donor were injected i. v. within 3 hours. Human immune cell engraftment was detected by flow cytometry 10-12 weeks after transplantation. All mice were housed and bred in a specific pathogen-free environment.

HIV-1 infection of humanized mice: The CCR5-tropic strain of HIV-1 (JR-CSF) was generated by transfection of 293T cells (ATCC) with plasmid containing full length HIV-1 (JR-CSF) genome. Humanized mice with stable human leukocyte reconstitution were anesthetized and infected with HIV-1 (JR-CSF) (10 ng p24/mouse) through retro-orbital injection. Humanized mice infected with 293T supernatant were used as mock control groups.

Development of anti-IFNAR1 blocking antibody: The generation of anti-IFNAR1 was performed as reported (Cheng et al., J Clin. Invest. 127:269 (2017)). Briefly, the mouse cell line L-929 transfected with the human IFNAR1 (extracellular domain and trans-membrane domain) expression plasmid was used as the immunogen for immunization with CpG-1826 as adjuvant. After 5 times immunization, the spleen cells were fused with mouse myeloma cell line SP2/0. 293 T cells transfected with the human IFNAR1 expression plasmid were used for screening the clones that could secret the IFNAR1 binding antibody by flow cytometry. Then, an IFN-I reporter 293T cell line which has been stably transfected with an mouse Mx2 promoter driven EGFP was used to screen antibody clones that could block the human IFNAR1 signaling.

In vivo IFNAR1 blocking antibody treatments: To confirm the in vivo neutralizing activity of mAb, humanized mice were treated i.p. with mAb 6 hours prior to R848 treatment. To block IFN-I signaling during chronic HIV-1 infection, humanized mice were treated i.p. with IFNAR1 blocking antibodies twice a week with the dose 400 μg/mouse at the first injection and 200 μg/mouse for the following treatments from 6 to 10 weeks post infection (wpi). Cohorts of mice were randomized into different treatment groups by level of HIV-1 RNA in plasma.

Quantification of mRNA expression by RT-PCR: RNA from PBMCs or whole splenocytes from humanized mice was isolated with the RNeasy plus extraction kit (Qiagen) and converted to cDNA by reverse transcription with random hexamers and SuperScript® III First-Strand Synthesis (Invitrogen). cDNA was then subjected to real-time PCR using gene-specific primers for:

| ISG15 | |
|---|---|
| (5′-CGCAGATCACCCAGAAGATCG-3′ | (SEQ ID NO: 1) |
| and | |
| 5′-TTCGTCGCATTTGTCCACCA-3′, | (SEQ ID NO: 2)) |
| MxA | |
| (5′-GGTGGTCCCCAGTAATGTGG-3′ | (SEQ ID NO: 22) |
| and | |
| 5′-CGTCAAGATTCCGATGGTCCT-3′, | (SEQ ID NO :23)) |
| OAS1 | |
| (5′-TGTCCAAGGTGGTAAAGGGTG-3′ | (SEQ ID NO: 24) |
| and | |
| 5′-CCGGCGATTTAACTGATCCTG-3′, | (SEQ ID NO: 25)) |
| IFITM3 | |
| (5′-ATGTCGTCTGGTCCCTGTTC-3′ | (SEQ ID NO: 26) |
| and | |
| 5′-GTCATGAGGATGCCCAGAAT-3′, | (SEQ ID NO: 27)) |
| Mx2 | |
| (5′-CAGAGGCAGCGGAATCGTAA-3′ | (SEQ ID NO: 3) |
| and | |
| 5′-TGAAGCTCTAGCTCGGTGTTC-3′, | (SEQ ID NO: 4)) |
| Trim22 | |
| (5′-CTGTCCTGTGTGTCAGACCAG-3′ | (SEQ ID NO: 28) |
| and | |
| 5′-TGTGGGCTCATCTTGACCTCT-3′, | (SEQ ID NO: 29)) |
| IRF7 | |
| (5′-GCTGGACGTGACCATCATGTA-3′ | (SEQ ID NO: 30) |
| and | |
| 5′-GGGCCGTATAGGAACGTGC-3′. | (SEQ ID NO: 31)) |

The IFN-stimulated gene expression levels were normalized to human CD45 controls. Results were calculated as a fold change in gene expression, relative to mock mice using the delta-delta Ct method of analysis (Schmittgen et al., Nature Protocols 3:1101 (2008)).

Flow cytometry: FITC-conjugated anti-human HLA-DR(L243), IFN-γ(4S.B3), PE-conjugated anti-human CD38 (HIT2), PerCP/Cy5.5-conjugated anti-human CD4 (RPA-T4), PE/Cy7-conjugated anti-human CD8 (HIT8a), IL-2 (MQ1-17H12), BV421- conjugated anti-human PD-1 (EH12.2H7), APC-conjugated anti-human Ki-67 (Ki-67) and APC/Cy7-conjugated anti-human CD45 (HI30) were purchased from Biolegend. FITC-conjugated anti-HIV-1 p24 were purchased from Beckman Coulter. PE-conjugated anti-human active caspase-3 (C92-605) was purchased from BD Pharmingen™. Pacific orange-conjugated anti-mouse CD45 (30-F11), PE/Texas red-conjugated anti-human CD3 (7D6) and LIVE/DEAD Fixable Yellow Dead Cell Stain Kit were purchased from Invitrogen. PE-conjugated A*02:01/SLYNTVATL Pentamer was purchased from PROIMMUNE.

For surface staining, single cell suspensions prepared from peripheral blood, spleen, or mesenteric lymph nodes of humanized mice were stained with surface markers and analyzed on a CyAn ADP flow cytometer (Dako). For intracellular staining, cells were first stained with surface markers, and then fixed and permeabilized with cytofix/cytoperm buffer (BD Bioscience), followed by intracellular staining. PE-conjugated anti-human active caspase-3 (C92-605) was used to detect intracellular activation of caspase-3. Fluorescent labeled inhibitors of caspases-1 probe assay (FLICA 660 Caspase-1 Assay Kit, ImmunoChemistry Technologies) were performed to determine intracellular activation of caspases-1. Data were analyzed using Summit4.3 software (Dako).

T cell stimulation and Intracellular cytokine staining: For non-specific stimulation, splenocytes from humanized mice were stimulated ex vivo with PMA (phorbol 12-myristate 13-acetate) (50 ng/ml) and ionomycin (1 uM) (sigma, St Louis, Mo.) for 4 hours in the presence of brefeldin A (Biolegend). For antigen-specific stimulation, splenocytes from humanized mice were stimulated ex vivo with peptide pools (2 μg/ml for each peptide) for HIV-1 GAG protein (PepMix™ HIV (GAG) Ultra, JPT Innovation Peptide Solutions) for 3 hours without brefeldin A and then 5 hours in the presence of brefeldin A. Cells were then fixed and permeabilized with cytofix/cytoperm buffer (BD Bioscience), and intracellular staining was then performed.

HIV-1 genomic RNA detection in plasma: HIV-1 RNA was purified from the plasma with the QIAampkit® Viral RNA Mini Kit. The RNA was then reverse transcribed and quantitatively detected by real time PCR using the TaqMan® Fast Virus 1-Step PCR kit (ThermoFisher Scientific). The primers used for detecting the HIV Gag gene were (5′-GGTGCGAGAGCGTCAGTATTAAG-3′ (SEQ ID NO: 17) and 5′-AGCTCCCTGCTTGCCCATA-3′(SEQ ID NO: 18)). The probe (FAM-AAAATTCGGTTAAGGCCAGGGGGAAAGAA-QSY7 (SEQ ID NO: 19)) used for detection was ordered from Applied Biosystems and the reactions were set up following manufacturer's guidelines and were run on the QuantStudio 6 Flex PCR system (Applied Biosystems).

Detection of cytokines in plasma: Human pan IFN-α (subtypes 1/13, 2, 4, 5, 6, 7, 8, 10, 14, 16 and 17) were detected by enzyme-linked immunosorbent assay using the human IFN-α pan ELISA kits purchased from Mabtech. Human IFN-β was detected by ELISA using the Verikine-HS human interferon beta serum ELISA kit. Human IFN-γ, TNF-α, IP-10, MCP-1 in plasma of humanized mice were detected by immunology multiplex assay (Luminex) (Millipore, Billerica, Mass., USA).

Ex vivo assays: Splenocytes from mock or HIV-1 persistent infected (15-20 weeks post infection) humanized mice were culture with 20 u/ml of IL-2 ex vivo in the present of mIgG2a control (10 μg/ml), α-IFNAR1 (10 μg/ml) or IFN-a (200 u/ml), caspase-3 inhibitor Z-DEVD-FMK(10 μM), caspase-1 inhibitor Ac-YVAD-CMK (50 μM) or DMSO as control for 10 days. Media were half-changed every 2-3 days. At day 10, the cells were counted and used for staining.

Statistical Analysis: In all other experiments, significance levels of data were determined by using Prism5 (GraphPad Software). Experiments were analyzed by two-tailed Student's t-test, or by one-way analysis of variance (ANOVA) and Bonferroni's post hoc test according to the assumptions of the test, as indicated for each experiment. Correlations between variables were evaluated using the Spearman rank correlation test. A P value less than 0.05 was considered significant. The number of animals and replicates is specified in each figure legend.

Study approval: Human fetal liver and thymus (gestational age of 16 to 20 weeks) were obtained from medically or elective indicated termination of pregnancy through a non-profit intermediary working with outpatient clinics (Advanced Bioscience Resources, Alameda, Calif.). Written informed consent of the maternal donors is obtained in all cases, under regulation governing the clinic. All animal studies were approved by the University of North Carolina Institutional Animal Care and Use Committee. The project was reviewed by the University's Office of Human Research Ethics, which has determined that this submission does not constitute human subjects research as defined under federal regulations [45 CFR 46.102 (d or f) and 21 CFR 56.102(c)(e)(1)]. All animal studies were approved by the University of North Carolina Institutional Animal Care and Use Committee (IACUC ID: 14-100) and were conducted following NIH guidelines for housing and care of laboratory animals.

### EXAMPLE 8

### Persistent HIV-1 Infection Leads to Sustained and Systemic IFN-I Induction

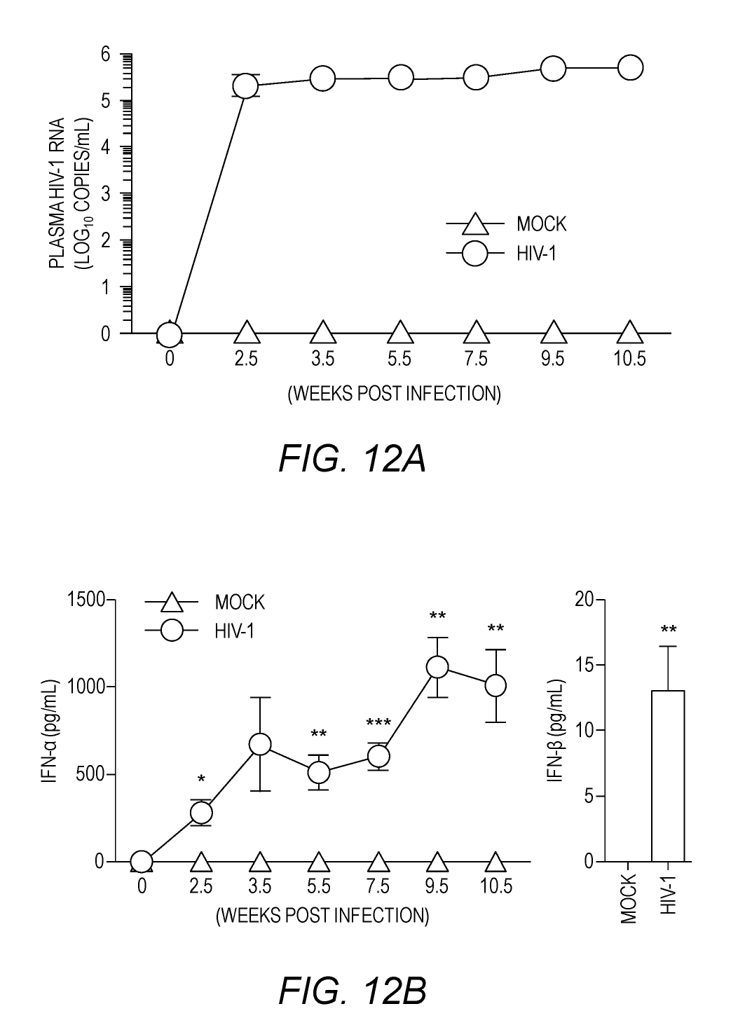

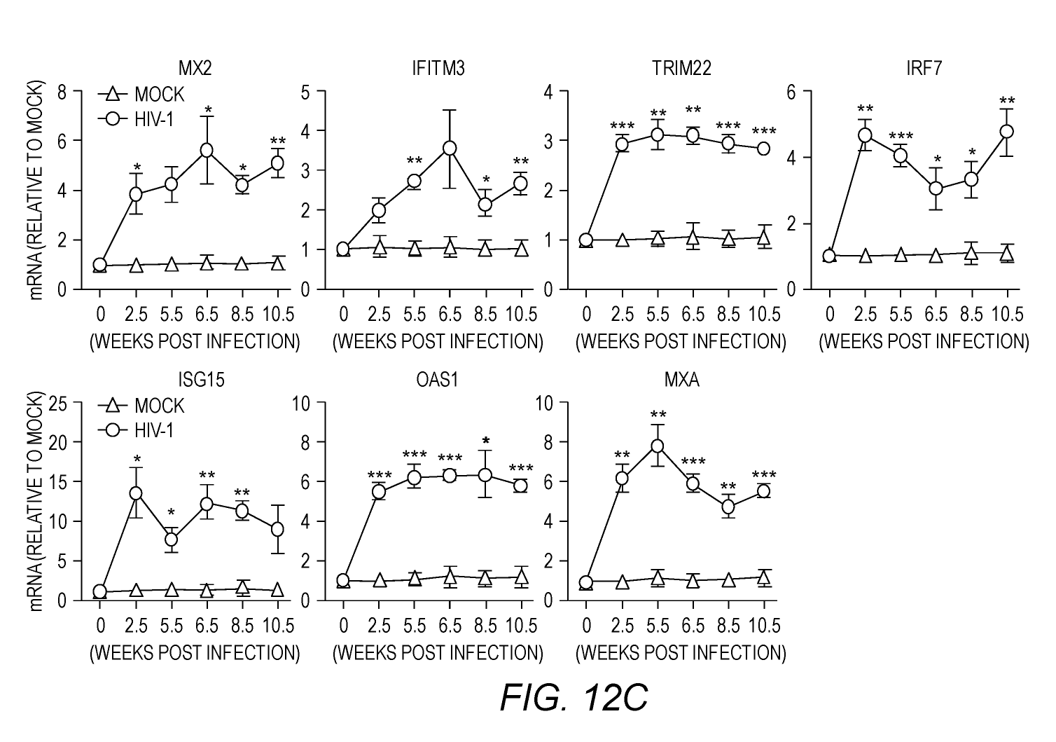

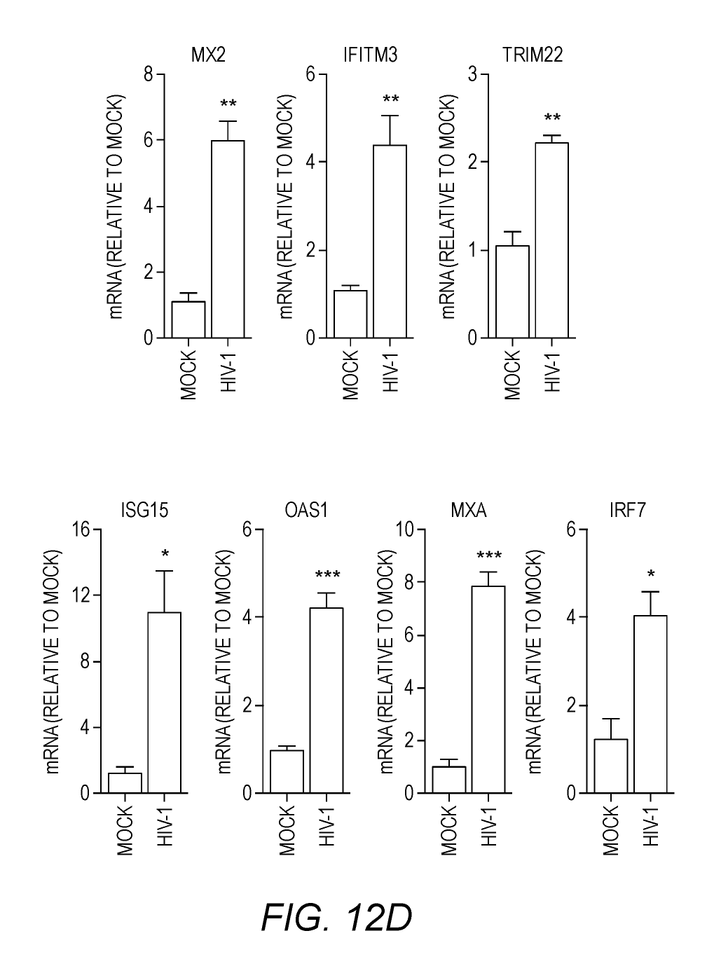

To functionally define the role of IFN-I in HIV-1 persistent infection and pathogenesis, humanized mice with a functional human immune system (hu-mice) were employed for modeling HIV-1 infection and immunopathogenesis (Shultz et al., Nature Rev. Immunol. 12:786 (2012); Zhang et al., Cell. Mol. Immunol. 9:237 (2012); Namikawa et al., Science 242:1684 (1988)). HIV-1 infection in hu-mice resulted in sustained plasma viremia (FIG. 12A). Similar to clinical observations (Bosinger et al., Curr. HIV/AIDS Rep. 12:41 (2015); Buimovici-Klein et al., Lancet 2(8345): 344 (1983)), HIV-1 infection in hu-mice also led to induction of IFN-I including IFN-α and IFN-β (FIG. 12B) and the induction of interferon stimulated-genes (ISGs), including Mx2, IFITM3, Trim22, ISG15, OAS1, MxA, and IFN regulatory factor 7 (IRF7) both in peripheral blood mononuclear cells (PBMCs) (FIG. 12C) and in spleen (FIG. 12D).

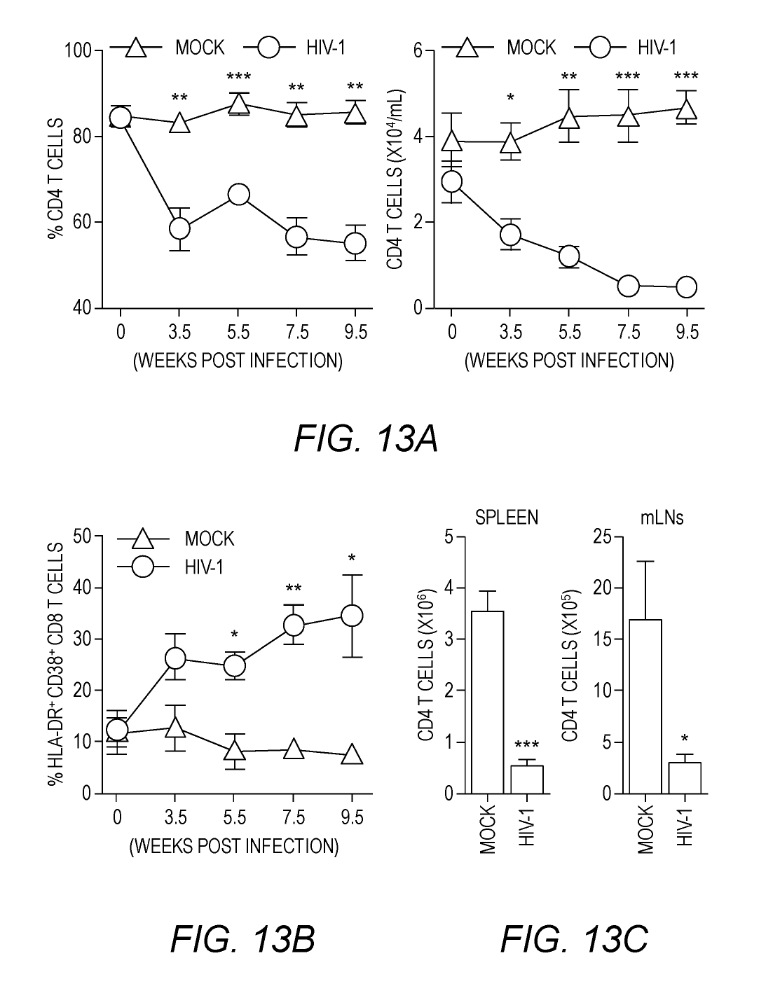

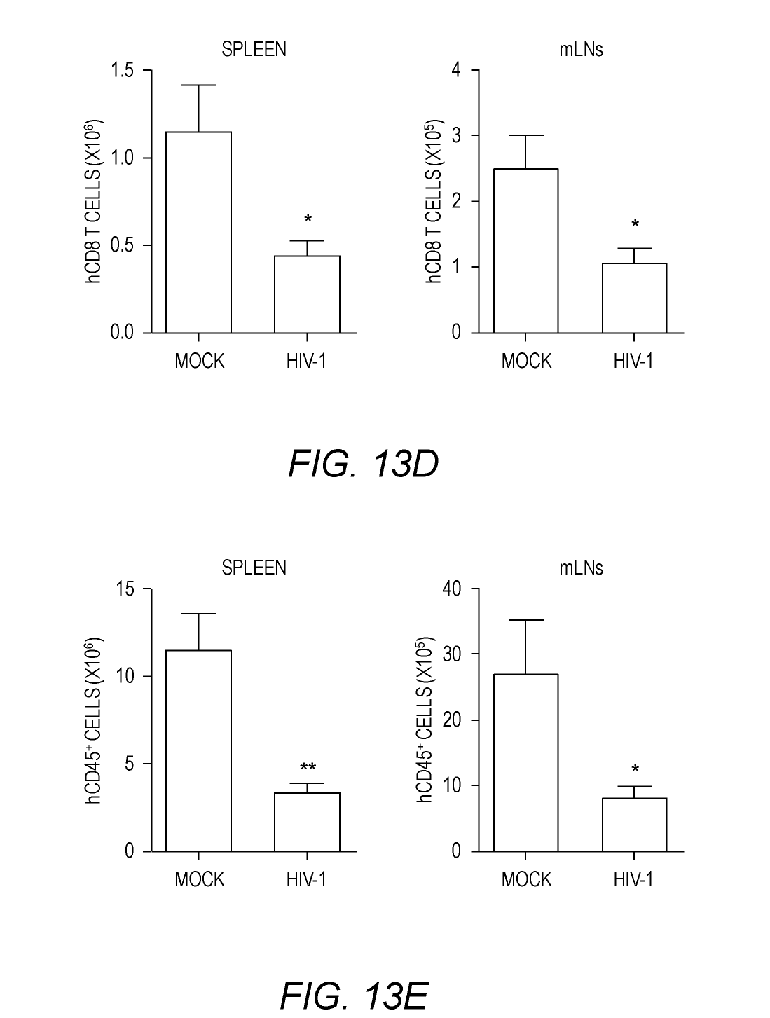

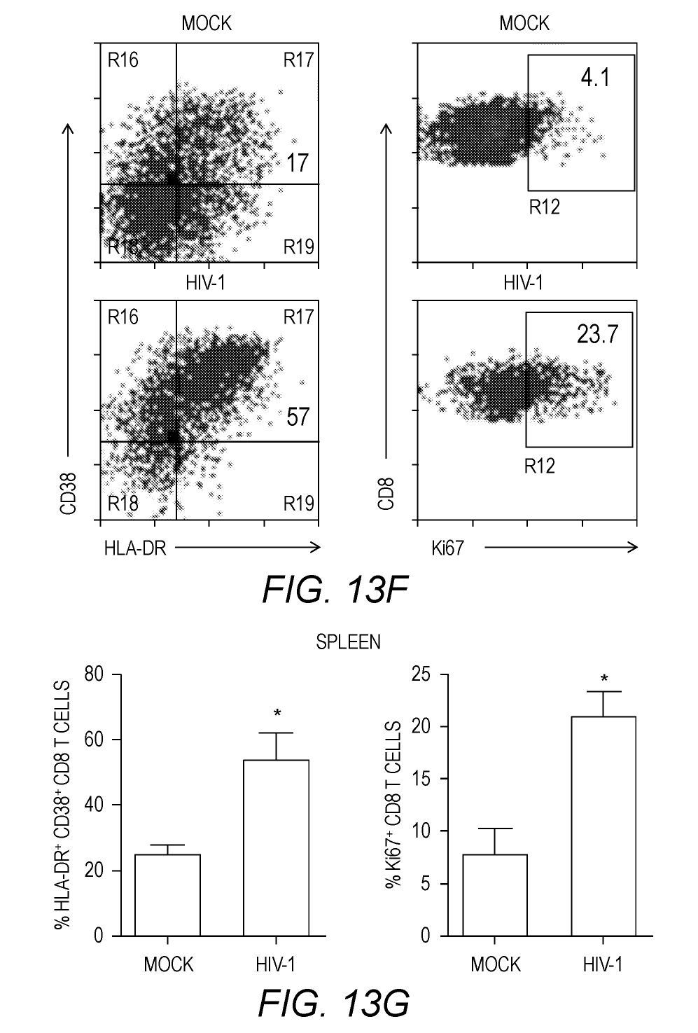

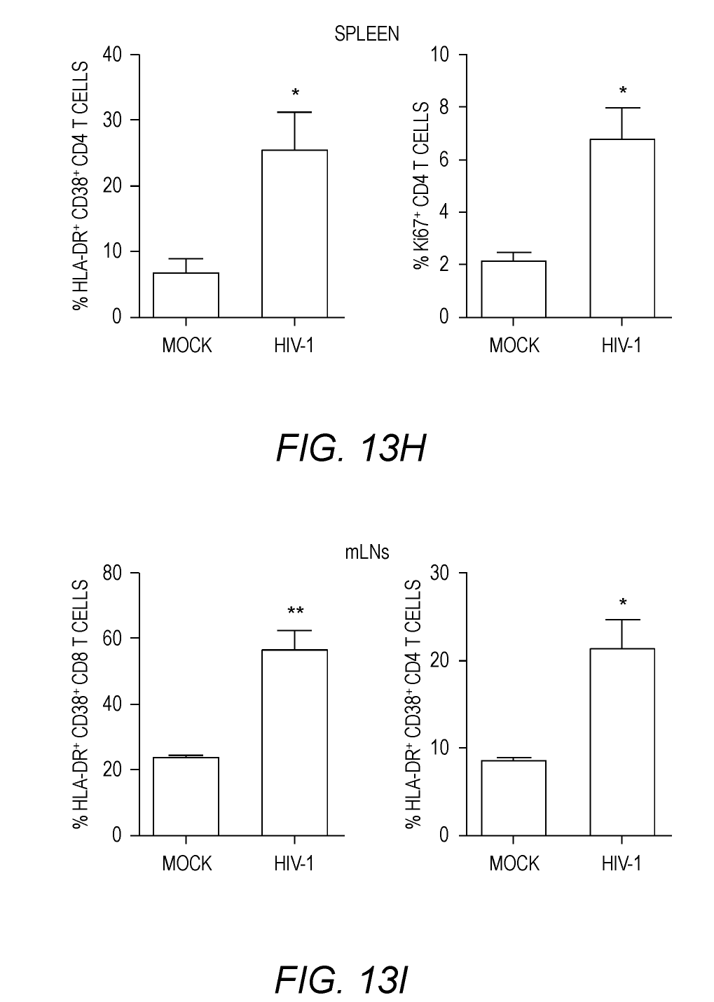

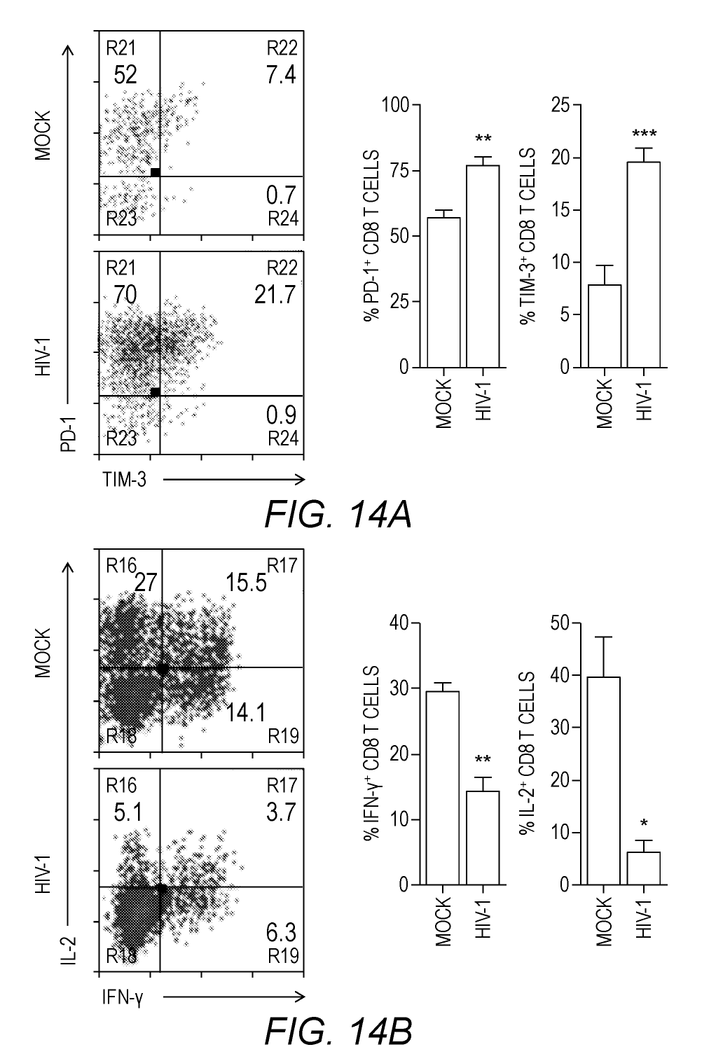

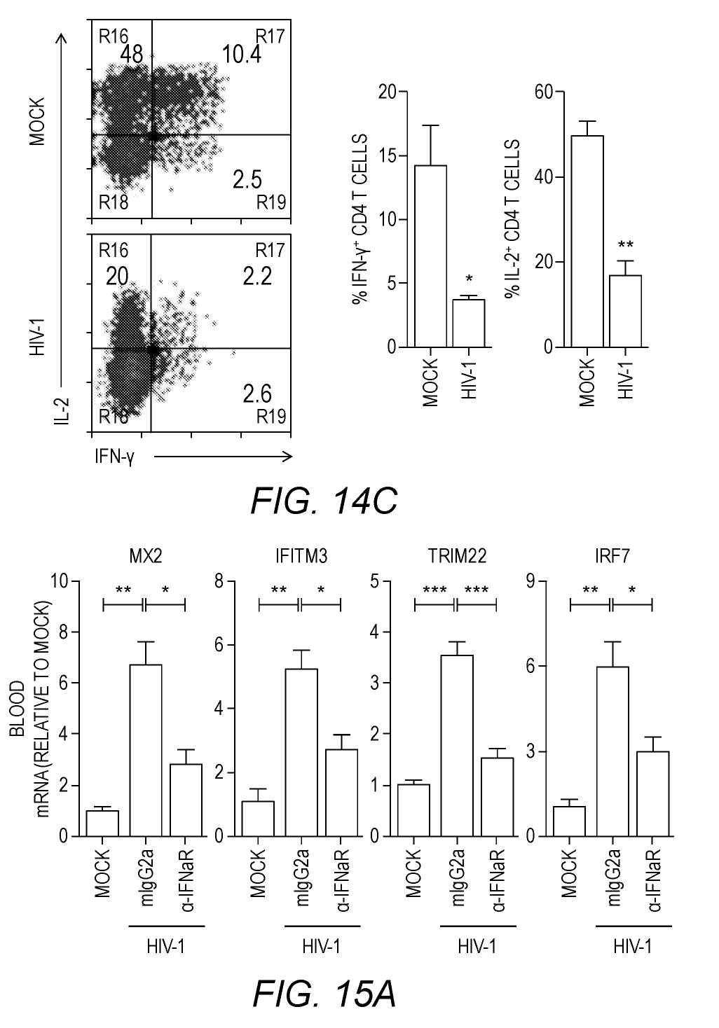

As observed in HIV-1 infected patients, HIV-1 infection in hu-mice induced severe depletion of human leukocytes including CD4 T cells in both peripheral blood and lymphoid organs (FIGS. 13A-13E). HIV-1 persistent infection also led to aberrant T-cell activation as indicated by enhanced expression of HLA-DR, CD38 and Ki67 (FIGS. 13F-13I). Additionally, human T cells in HIV-1 infected mice showed increased expression of exhaustion markers PD-1 and TIM-3 (FIG. 14A), associated with impaired T-cell functions as indicated by decreased capacity to produce IFN-α and IL-2 by both CD8 and CD4 T cells upon PMA/ionomycin stimulation (FIGS. 14B-14C). Therefore, persistent HIV-1 infection in hu-mice led to systemic and sustained IFN-I signaling associated with CD4 T cell depletion, aberrant immune activation and T cell exhaustion.

### EXAMPLE 9

### IFNAR1 Blockade during Persistent HIV-1 Infection Elevates HIV-1 Replication

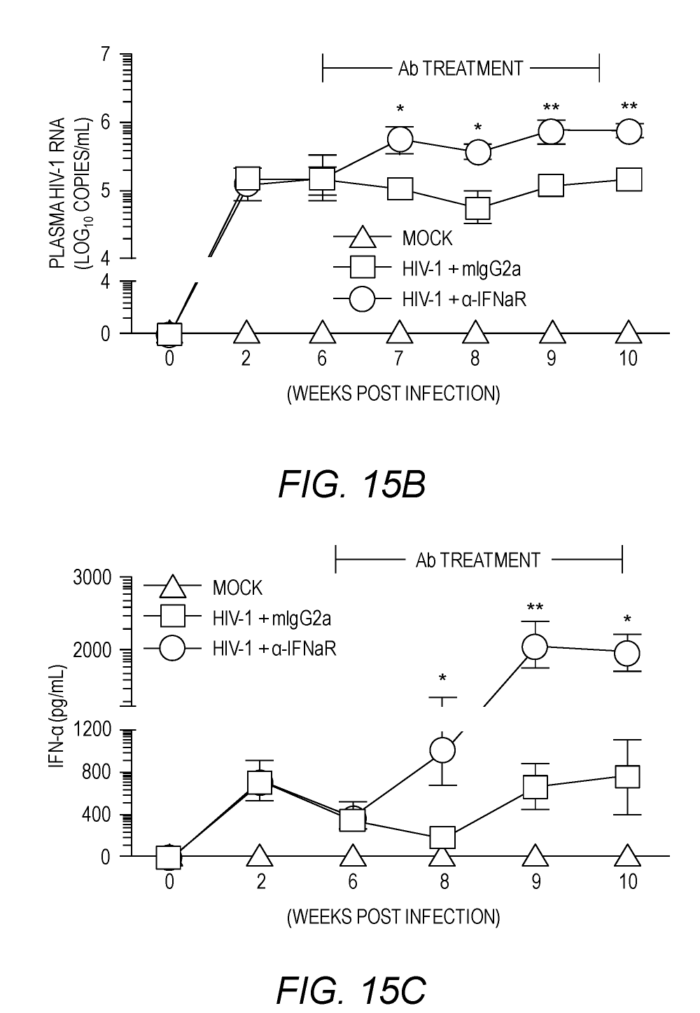

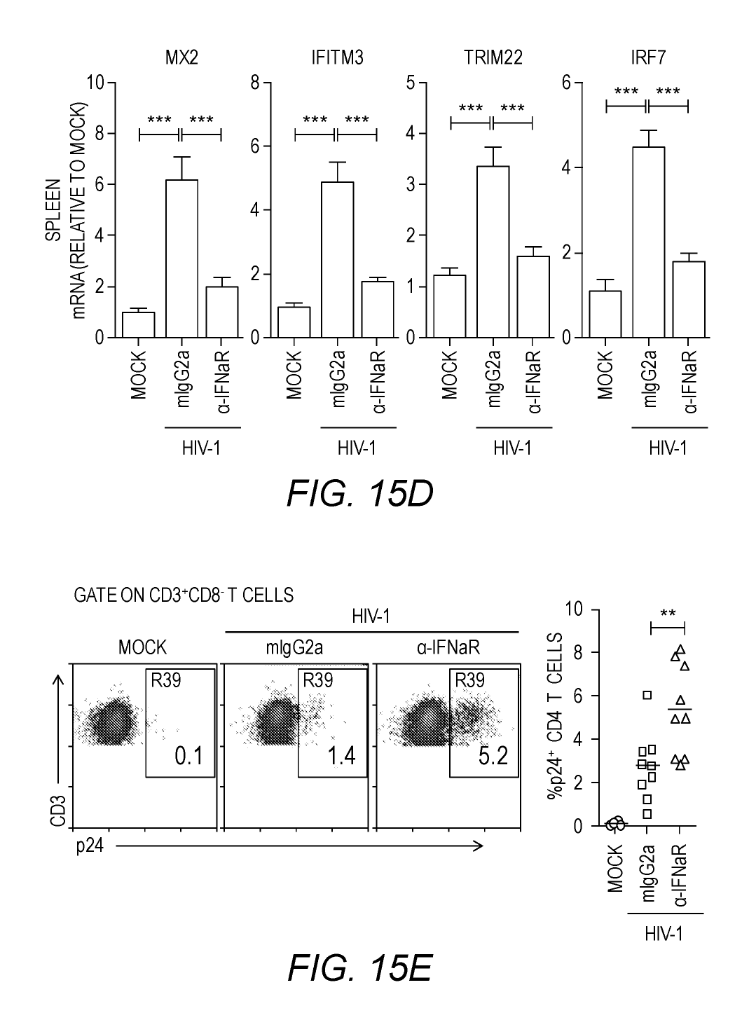

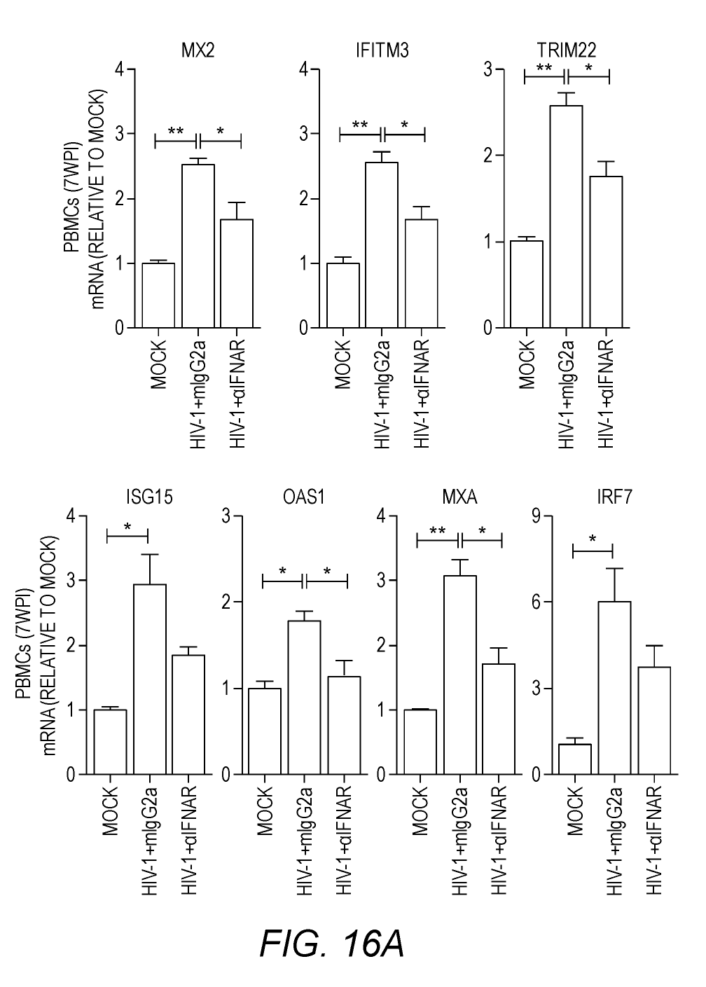

To define the role of IFN-I during persistent HIV-1 infection in vivo, humanized mice were infected and HIV-1 infected hu-mice were treated with IFNAR1 blocking mAb from 6 to 10 weeks post infection (wpi). As expected, IFNAR1 mAb treatment blocked ISGs, including the ISGs with anti-HIV-1 function such as Mx2 (Kane et al., Nature 502:563 (2013); Goujon et al., Nature 502:559 (2013)) and IFITM3 (Lu et al., J. Vir. 85:2126 (2011); Yu et al., Cell Rep. 13:145 (2015)), expression in PBMCs of infected hu-mice (FIG. 15A and FIGS. 16A-16B). It was found that HIV-1 replication increased 8-fold within one week after IFNAR1 blockade and sustained at higher levels than control mice from 7-10 wpi (FIG. 15B). IFNAR1 blocking resulted in higher IFN-α levels in peripheral blood (FIG. 15C), correlated with higher HIV-1 viremia in α-IFNAR1 treated mice. At termination (10 wpi), IFNAR1 blockade also blocked ISGs expression (FIG. 15D and FIG. 16C) and increased HIV-1 replication in spleen (FIG. 15E). These results indicate that, during persistent HIV-1 infection, IFN-I still contributes to the suppression of HIV-1 replication.

### EXAMPLE 10

### Elevated HIV-1 Replication Correlates with Higher Levels of T Cell Activation and Pro-Inflammatory Cytokine Production after IFNAR1 Blockade

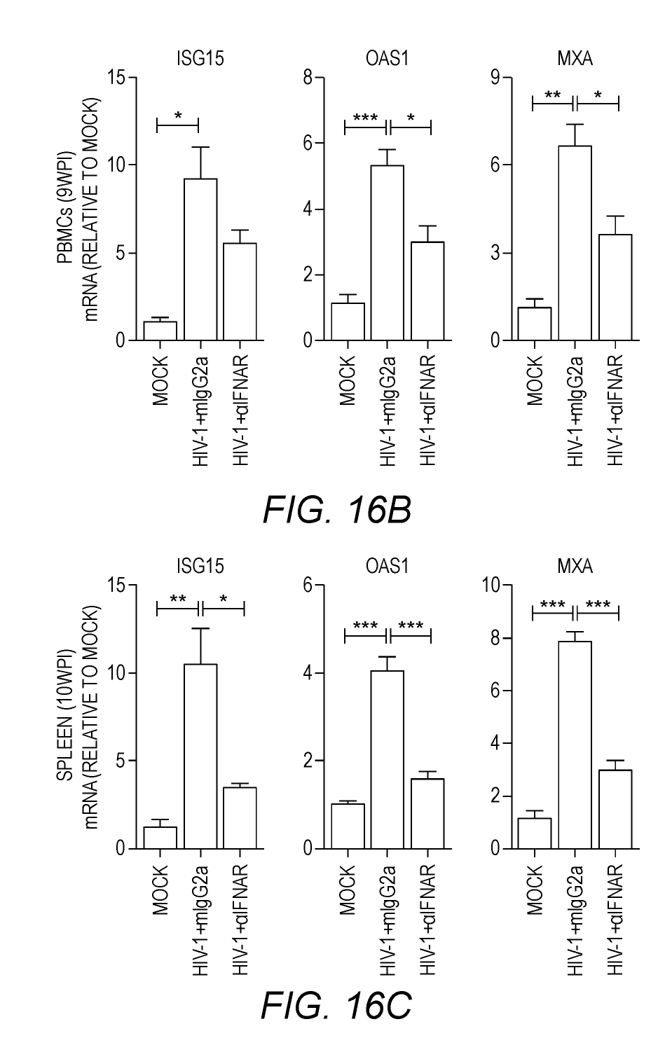

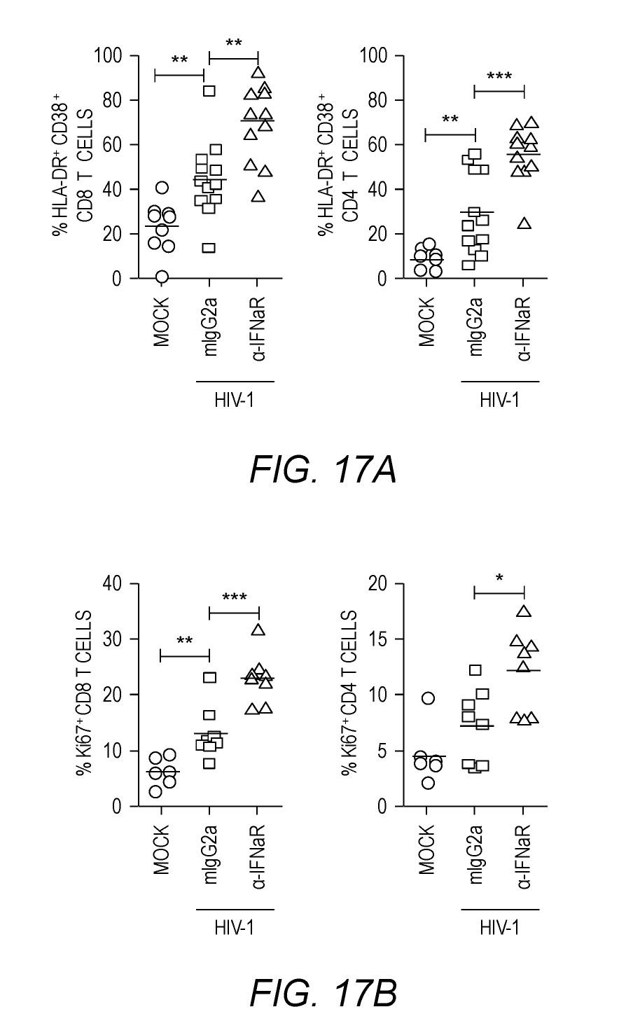

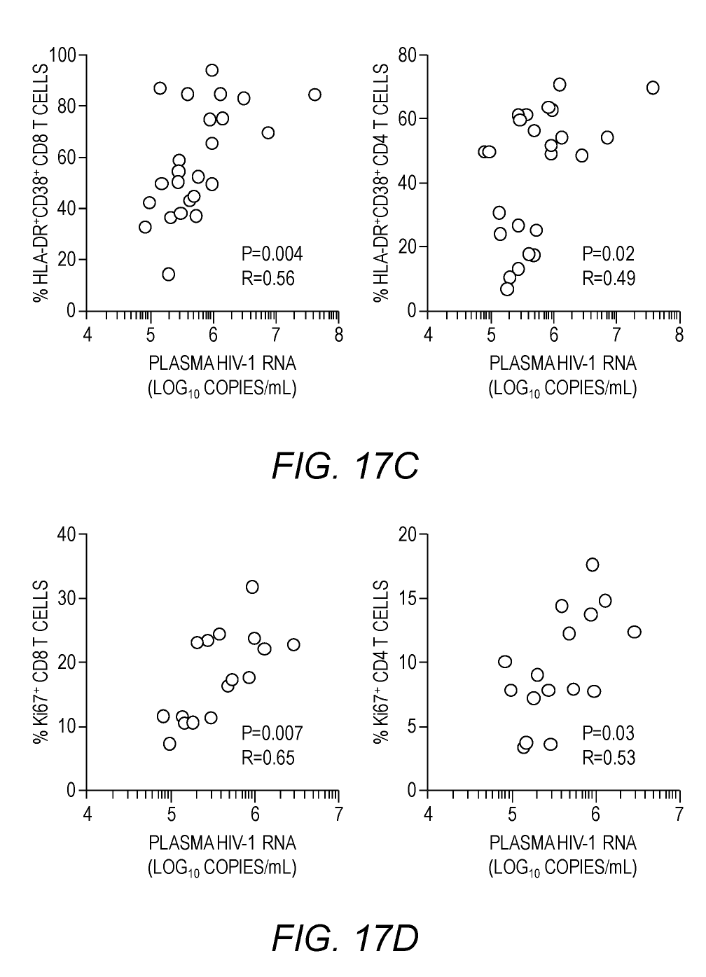

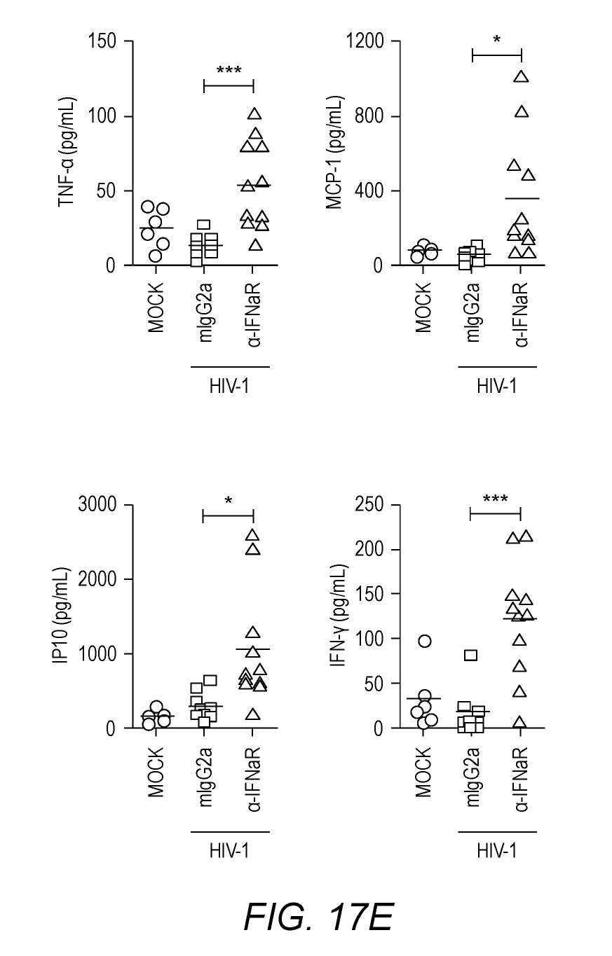

It was found that the frequencies of CD38/DR- and Ki67-positive CD8 and CD4 T cells were increased by blocking IFNAR1 (FIGS. 17A-17B). The expression level of CD38/DR and Ki67 on T cells were positively correlated with HIV-1 viremia in plasma (FIGS. 17C-17D). IFNAR1 blockade also led to increased expression of TNF-α, MCP-1, IP-10 and IFN-α in plasma of HIV-1 infected hu-mice (FIG. 17E). These results indicate that, in the absence of IFN-I signaling, elevated viral replication led to higher immune activation, correlated with elevated levels of HIV-1 replication. Thus, IFN-I signaling is not essential for the aberrant T-cell immune activation during chronic phase of HIV-1 infection.

### EXAMPLE 11

### IFNAR1 Blockade during Persistent HIV-1 Infection Rescues Human T Cells, including HIV-Specific T Cells

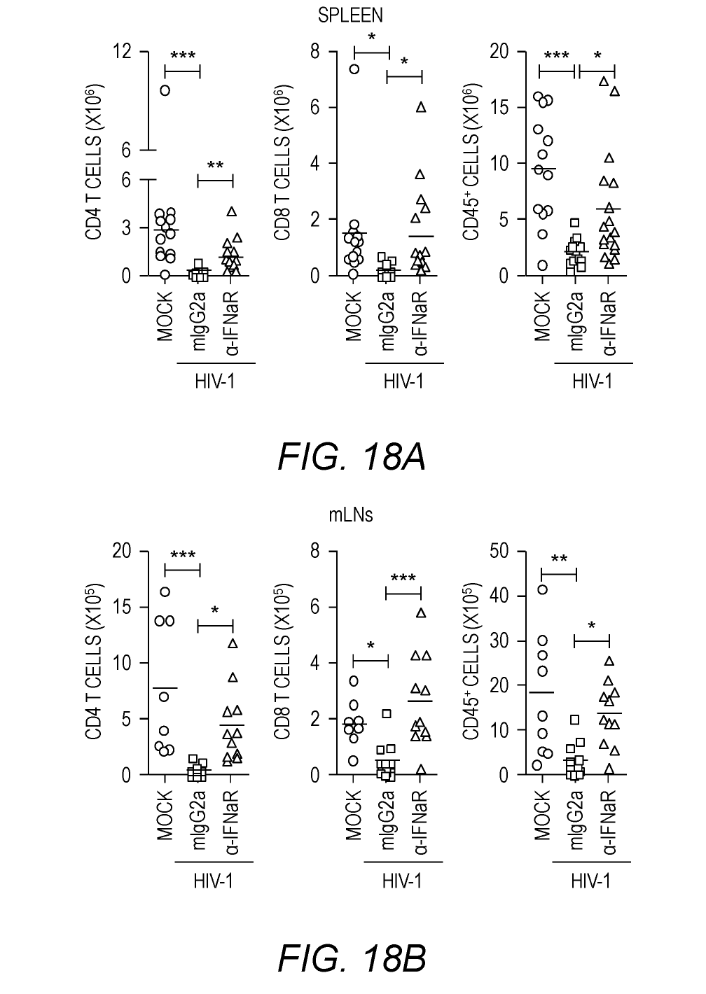

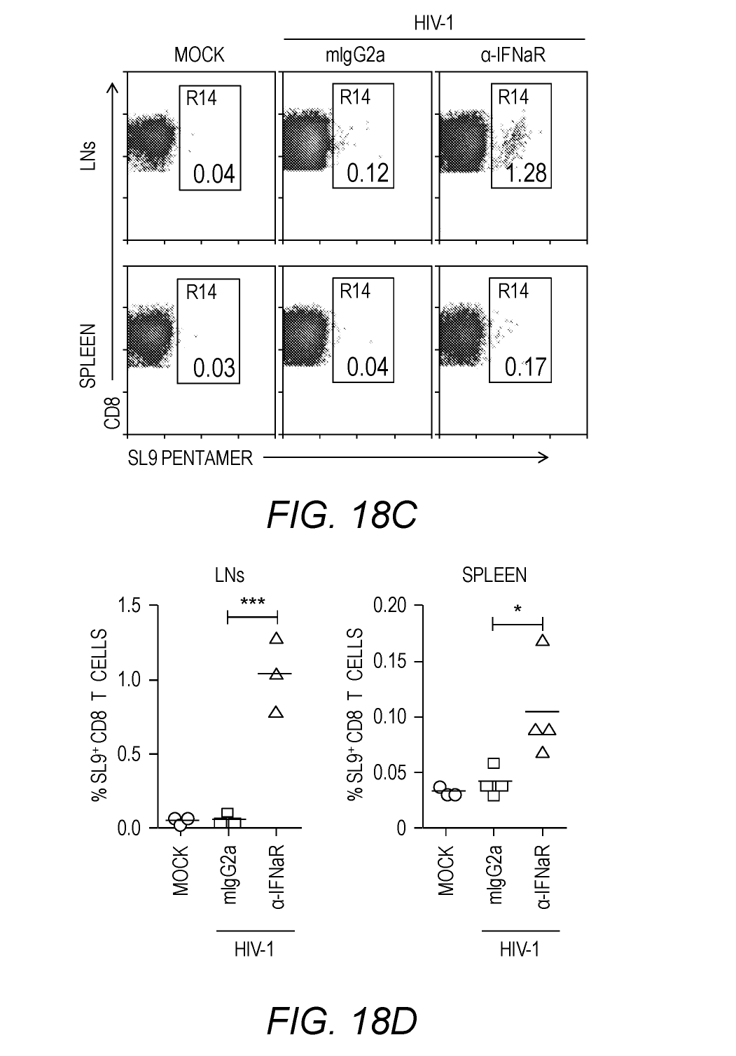

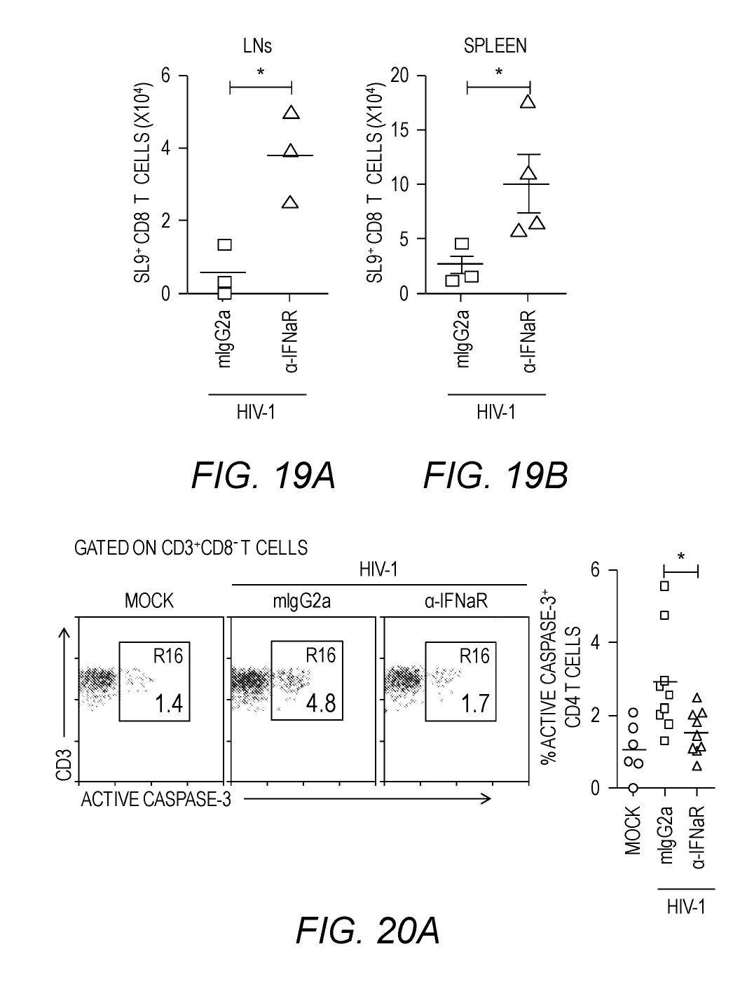

Systemic chronic immune activation is associated with CD4 T-cell depletion (Paiardini et al., Immunol. Rev. 254:78 (2013); Moir et al., Annu. Rev. Pathol. 6:223 (2011)). However, the causal link between immune activation and CD4 T-cell loss is unclear (Moir et al., Annu. Rev. Pathol. 6:223 (2011)). The effect of IFNAR1 blockade on HIV-1 induced pathogenesis was analyzed, including CD4 T-cell depletion. Surprisingly, despite increased HIV-1 replication and T cell activation, the number of human CD4 T cells in the spleen and mesenteric lymph nodes (mLNs) was significantly increased in hu-mice with IFNAR1 blockade (FIGS. 18A-18B). IFNAR1 blockade also rescued human CD8 T cells and total human leukocytes in lymphoid organs (FIGS. 18A-18B). Furthermore, it was found that IFNAR1 blockade significantly increased the percentage and number of HLA-A2/SL-9 (an epitope consisting of amino acids 77-85 of HIV-1 p17 protein) pentamer-specific CD8 T cells in lymphoid organs (FIGS. 18C-18D and FIGS. 19A-19B). These results suggest that, in the absence of IFN-I signaling, elevated immune activation does not lead to T cell depletion. IFN-I signaling thus contributes to the depletion of T cells during persistent HIV-1 infection.

### EXAMPLE 12

### IFNAR1 Blockade Protects HIV-1 Induced Apoptosis of CD4 T Cells

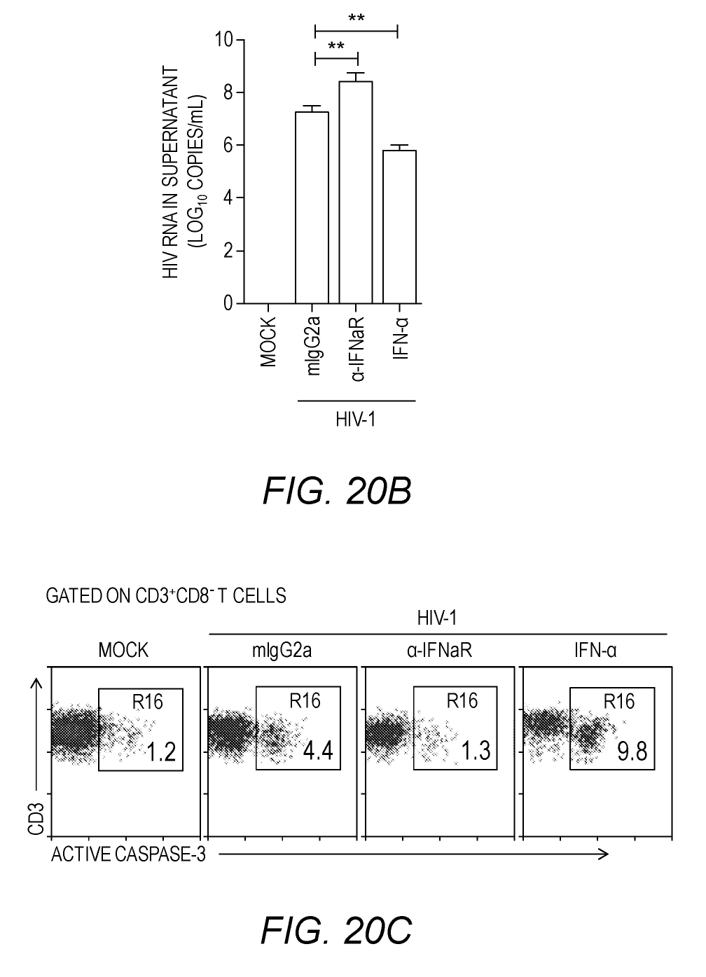

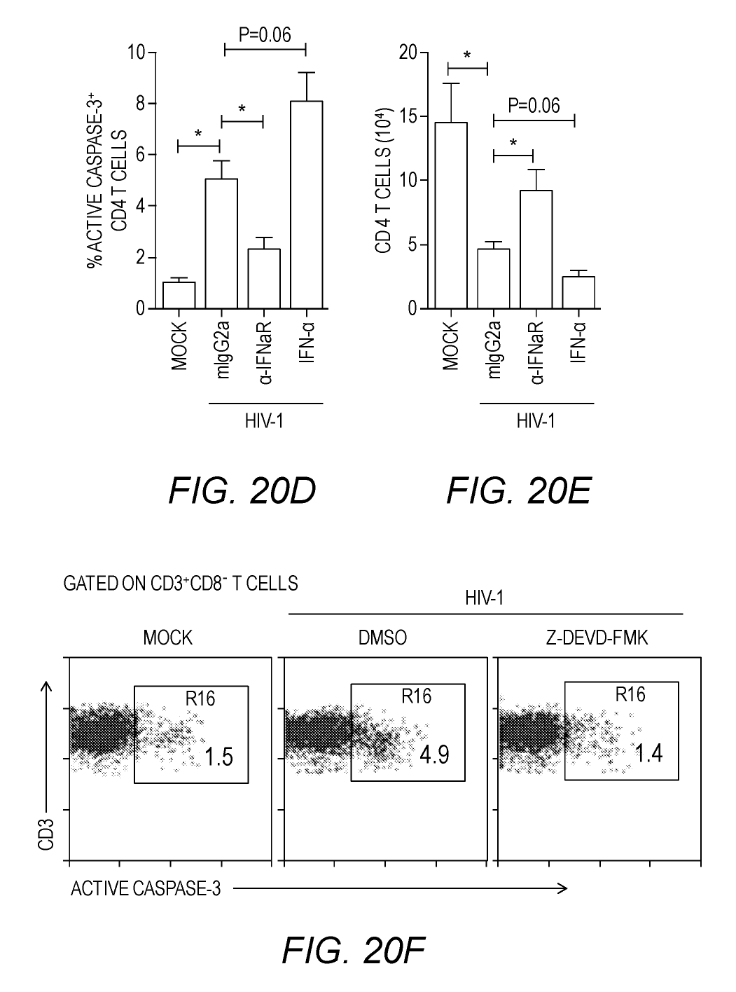

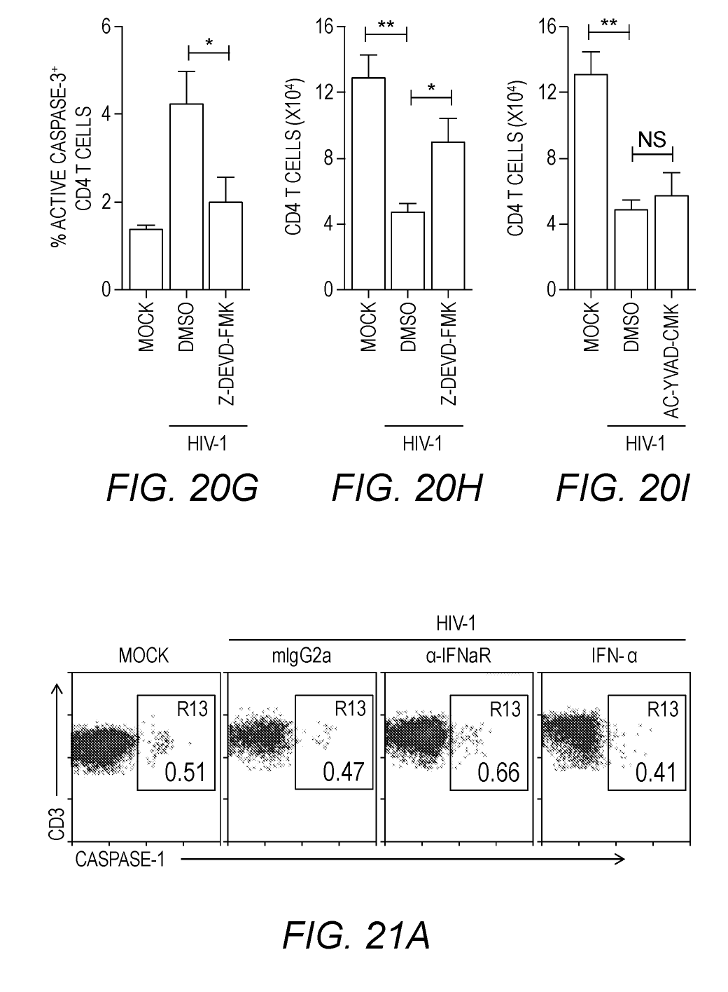

Elevated apoptosis is correlated with CD4 T cell depletion during HIV-1 infection (Finkel et al., Nature Med. 1:129 (1995); Herbeuval et al., Blood 106:3524 (2005); Fraietta et al., PLoS Pathogens 9:e1003658 (2013)). Higher levels of active caspase-3 were detected in CD4 T cells after HIV-1 persistent infection in hu-mice (FIG. 20A). IFNAR1 blockade reduced the level of active caspase-3 in CD4 T cells (FIG. 20A). The result suggests that blockade of IFN-I signaling prevents HIV-1 induced CD4 T-cell apoptosis.

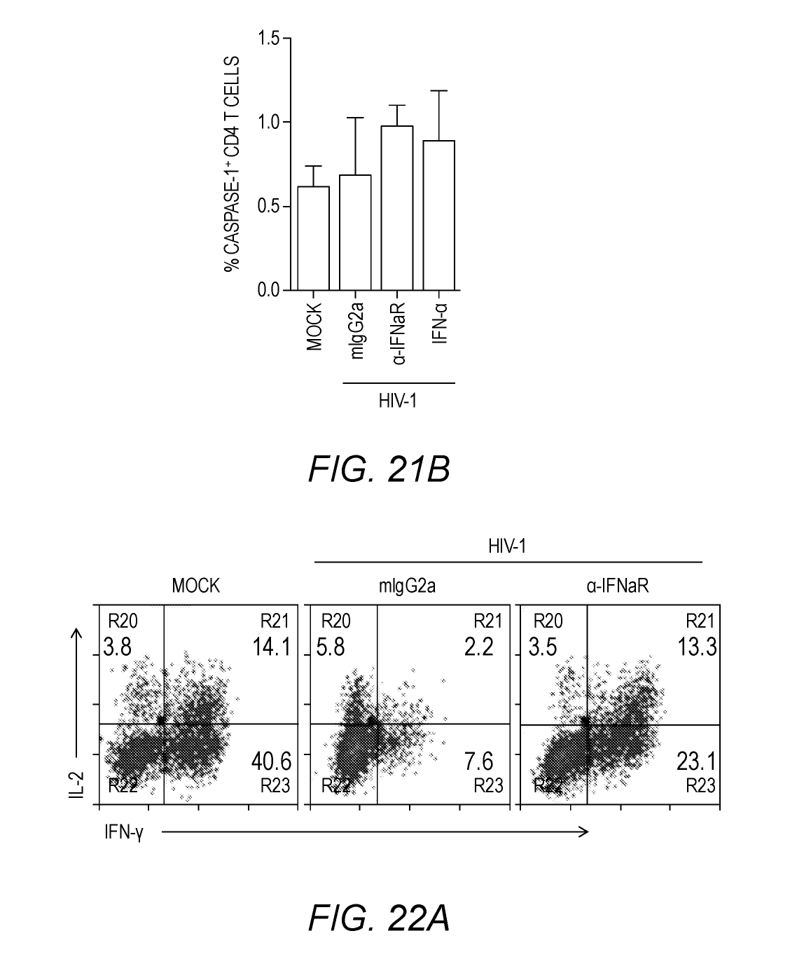

The ex vivo culture system was used to further investigate how IFN-I signaling leads to CD4 T cell depletion (Zhang et al., J Clin. Invest. 125:3692 (2015)). Splenocytes were isolated from hu-mice persistently infected with HIV-1 and cultured ex vivo in the presence of isotype control Ab, α-IFNAR1 mAb or IFN-α. IFNAR1 blockade increased HIV-1 replication and exogenous IFN-α inhibited HIV-1 replication (FIG. 20B). It was found that, as in lymphoid organs in vivo, CD4 T cells from HIV-infected mice showed higher levels of active caspase-3 (FIGS. 20C-20D). IFNAR1 blockade reduced the level of active caspase-3 in CD4 T cells (FIGS. 20C-20D). Accordingly, exogenous IFN-α added to the culture system further increased the level of active caspase-3 (FIGS. 20C-20D). Thus, IFNAR1 blockade rescued HIV-1 induced CD4 T cell depletion despite elevated HIV-1 replication, and exogenous IFN-α accelerated CD4 T cell depletion (FIG. 20E) although it inhibit HIV-1 replication. In addition, inhibition of the activity of active caspase-3 by a specific inhibitor Z-DEVD-FMK also reduced HIV-1 induced CD4 T cell apoptosis and rescued CD4 T cell number (FIGS. 20E-20H). In contrast, caspase 1 expression in CD4 T cells did not increase in HIV-1 infected sample (FIGS. 21A-21B) and inhibition of caspase-1 activity did not prevent CD4 T cell depletion (FIG. 20I). Together, these results indicate that sustained IFN-I signaling contributes to apoptosis of CD4 T cells during persistent HIV-1 infection.

### EXAMPLE 13

### IFNAR1 Blockade Rescues Function of Human T Cells During Persistent HIV-1 Infection

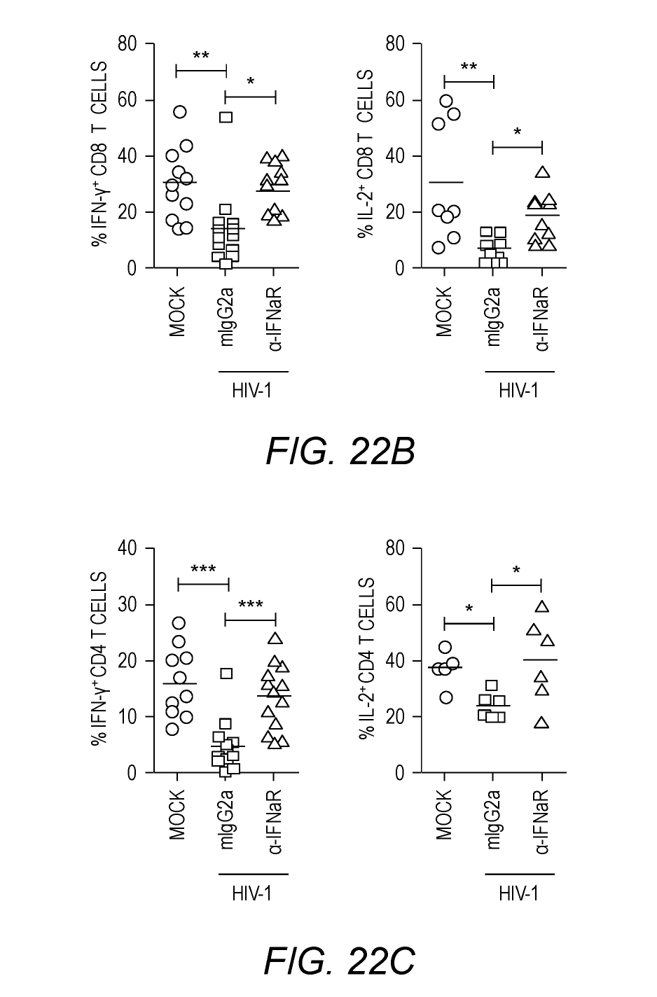

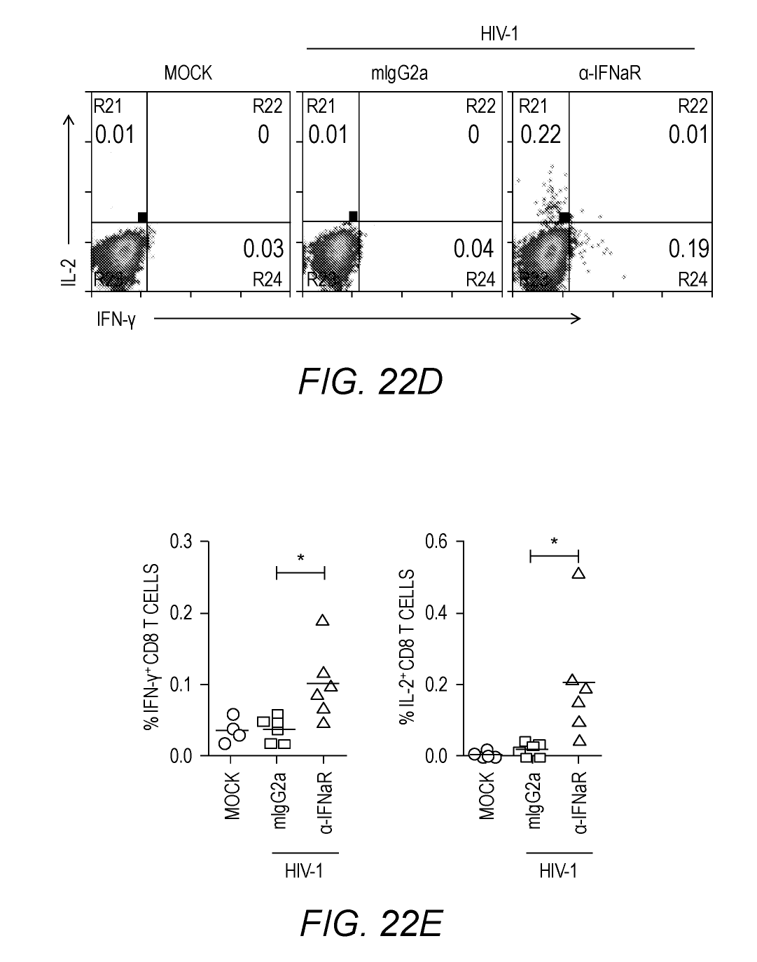

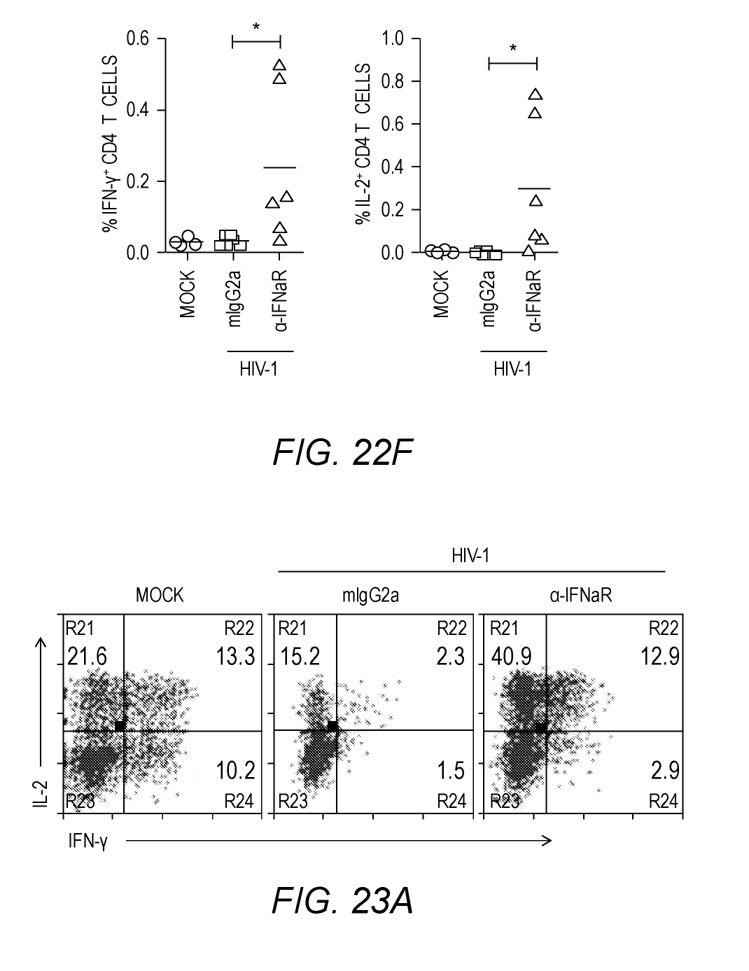

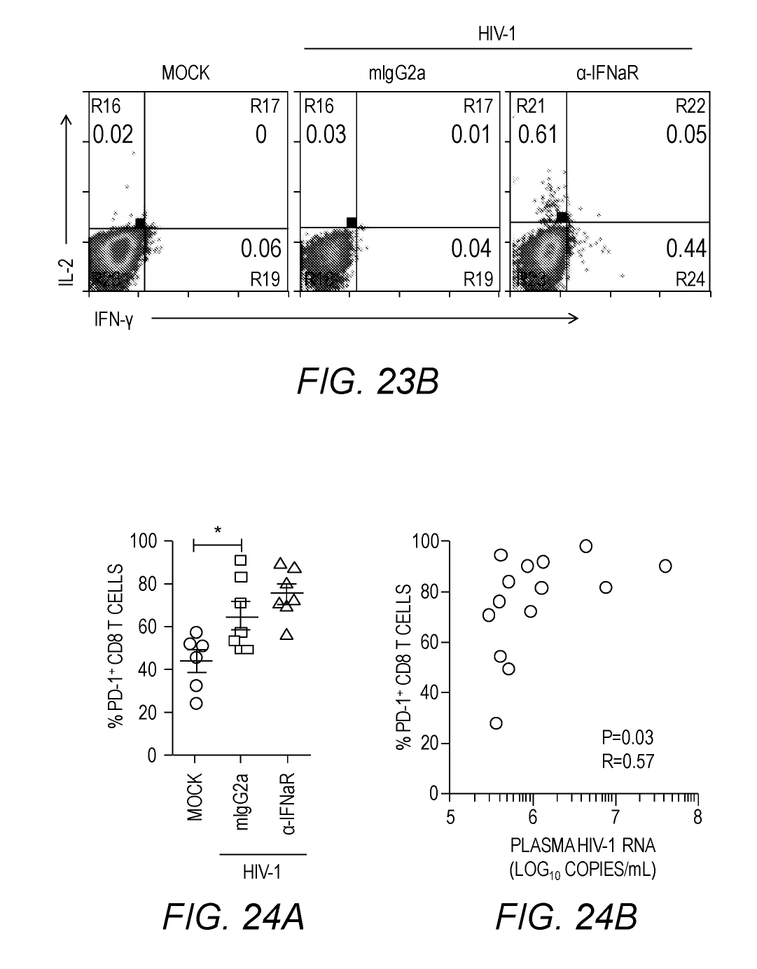

Next, the contribution of IFN-I signaling to HIV-1 induced functional impairment of T cells was analyzed. It was found that IFNAR1 blockade restored the ability of both CD8 and CD4 T cells to produce IFN-α and IL-2 upon PMA/Ionomycin stimulation (FIGS. 22A-22C and FIG. 23A). IFNAR1 blockade did not reduce the expression level of PD-1 on T cells (FIG. 24A), which was correlated with more viral replication (FIG. 24B). The function of HIV-1-specific T cells after IFNAR1 blockade was further analyzed. When stimulated with HIV-1 Gag peptide pool ex vivo, both CD8 and CD4 T cells from hu-mice with IFNAR1 blockade produced significantly higher levels of IFN-α and IL-2 (FIG. 22D-22F and FIG. 23B), indicating that IFNAR1 blockade also rescued functional responses of HIV-1 specific T cells in persistently infected hosts. Together, IFNAR1 blockade during persistent HIV-1 infection rescues both number and function of human T cells, including HIV-1 specific T cells.

The contribution of IFN-I signaling to HIV-1 replication and immunopathogenesis during chronic infection is not clearly defined (Doyle et al., Nat. Rev. Microbiol. 13:403 (2015); Bosinger et al., Curr. HIV/AIDS Rep. 12:41 (2015)), although persistent activation of pDC has been reported to contribute to HIV-induced immunopathogenesis (Zhang et al., J. Clin. Invest. 125:3692 (2015); Li et al., PLoS Pathogens 10:e1004291 (2014)). Using an α-IFNAR1 mAb to block IFN-I signaling in vivo, it was shown here that IFNAR1 blockade during persistent HIV-1 infection increased HIV-1 replication in hu-mice. It was discovered that IFNAR1 blockade rescued human T-cell numerically and functionally despite elevated HIV-1 replication and T cell activation. These results agree with recent findings showing that persistent IFN-I signaling plays a detrimental role during chronic LCMV infection and blocking IFN-I signaling by IFNAR antibody could reverse T cell exhaustion and enhance antiviral immune response (Wilson et al., Science 340:202 (2013); Teijaro et al., Science 340:207 (2013)).

During the chronic phase of HIV-1 infection, a positive correlation between the expression of ISGs and HIV-1 viremia has been reported (Hardy et al., PloS One 8:e56527 (2013); Rotger et al., PLoS Pathogens 6:e1000781 (2010)). Persistent ISG expression is also associated with pathogenic SIV infection (Bosinger et al., Invest. 119:3556 (2009); Jacquelin et al., J. Clin. Invest. 119:3544 (2009); Favre et al., PLoS Pathogens 5:e1000295 (2009); Lederer et al., PLoS Pathogens 5:e1000296 (2009)). However, the role of IFN-I signaling in HIV-1 infection and disease progression is not clearly defined. It was found here that blocking endogenous IFN-I signaling during persistent HIV-1 infection by α-IFNAR1 increased viral replication. The result is consistent with the previous finding that depletion of pDCs, the major IFN-I producing cells during HIV-1 infection, increased HIV-1 replication (Li et al., PLoS Pathogens 10:e1004291 (2014)). Thus, it was demonstrated that HIV-1 is still sensitive to IFN-I during persistent HIV-1 infection.

HIV-1 disease progression is associated with depletion of human leukocytes including human CD4 T cells. Although several reports show that persistent ISG induction is correlated with disease progression in HIV-1 infected patients (Rotger et al., PLoS Pathogens 6:e1000781 (2010); Hyrcza et al., J. Virol. 81:3477 (2007); Sedaghat et al., J. Virol. 82:1870 (2008)), the direct causal link between IFN-I signaling and CD4 T cell depletion is not clearly established. In the present study, it was found that IFNAR1 blockade rescued both number and function of human T cells, and prevented HIV-1 induced CD4 T cell apoptosis, in spite of higher levels of virus replication. Thus, sustained IFN-I signaling plays a major role in CD4 T cell depletion during persistent HIV-1 infection. Interestingly, it was found that IFNAR1 blockade increased the expression of activation marker CD38/HLA-DR and Ki67 on both CD4 and CD8 T cells which is positively correlated with HIV-1 viremia. These data also indicate that IFN-I is not essential for HIV-induced aberrant immune activation. In the absence of IFN-I signaling, higher levels of HIV-1 viremia or aberrant immune activation failed to lead to CD4 T cell depletion. Thus, it is concluded that IFN-I is critical to the death of human T cells during chronic HIV-1 infection.

IFN-I also plays an important role in modulating T cell function (Crouse et al., Nature Rev. Immunol. 15:231 (2015)). During chronic lymphocytic choriomeningitis virus (LCMV) infection in mice, blocking IFN-I signaling by IFNAR1 antibody can enhance antiviral immune response and prevent or accelerate clearance of persistent LCMV infection in mice (Wilson et al., Science 340:202 (2013); Teijaro et al., Science 340:207 (2013)). It was demonstrated here that during chronic HIV-1 infection in hu-mice, blocking IFN-I signaling rescued the HIV-1 specific T cell function. IFNAR blockade may lead to the improvement in anti-HIV T cell responses by several mechanisms. First, it has been reported that sustained IFN-I signaling leads to the expression of the negative immune regulators IL-10 and PD-L1 on antigen presenting cells during LCMV infection in mice. Blockade of IFN-I signaling improves DCs functions may contribute to the restoration of anti-LCMV T cell response (Wilson et al., Science 340:202 (2013); Teijaro et al., Science 340:207 (2013); Crouse et al, Nature Rev. Immunol. 15:231 (2015)). Second, IFNAR signaling in T cells may have direct anti-proliferative and pro-apoptotic effects (Fraietta et al., PLoS Pathogens 9:e1003658 (2013); Crouse et al., Nature Rev. Immunol. 15:231 (2015); Dondi et al., J. Immunol. 170:749 (2003); Bromberg et al., Proc. Natl. Acad. Sci. USA 93:7673 (1996)). In addition, the preservation of helper CD4 T cell by IFNAR blocking may also help to enhance anti-HIV CD8 T cell activity. However, blockade of IFNAR in humanized mice also led to elevated HIV-1 replication. This may be due to the loss of innate anti-HIV-1 effect of IFN-I after IFNAR blocking that overcome the benefit of reversed T cell response in viral inhibition. It is also important to point out that the human immunity developed in humanized mice is not fully functional as found in immunocompetent hosts (Shultz et al., Nature Rev. Immunol. 12:786 (2012); Zhang et al., Cell. Mol. Immunol. 9:237 (2012)). The restored anti-HIV-1 T cell immune response by IFNAR blockade in humanized mice may not be robust enough to control the HIV-1 replication.

A recent report showed that blockade of IFNAR2 during chronic phase of HIV-1 infection in humanized mice led to decreased viral replication, diminished HIV-driven immune activation and restored HIV-specific CD8 T cell function (Zhen et al., J. Clin. Invest. 127:260 (2017)). The difference between the two studies is that Zhen et al. used antibody to block IFNAR2 while antibody to block IFNAR1 was used here. As reported previously, IFN-β can specifically and uniquely bind to IFNAR1 to initiate downstream signaling in an IFNAR2-independent manner (de Weerd et al., Nature Immunol. 14:901 (2013)). It is thus likely that blocking IFNAR1, but not IFNAR2, can completely block all types of IFN-I signaling. The remaining IFN-I signaling which cannot be blocked by IFNAR2 antibody in the report by Zhen et al. may still contribute to the suppression of HIV-1 replication. Interestingly, HIV-specific CD8 T cell function was restored either by IFNAR1 blockade or by IFNAR2 blockade. The reversed anti-HIV T cell function may synergize with the remaining IFN-I signaling to further control HIV-1 replication (Zhen et al., J. Clin. Invest. 127:260 (2017)). Thus, it will be important to dissect the specific IFN-I subtypes, their signaling pathways and ISGs that contribute to viral inhibition and host immunity impairment during persistent HIV-1 infection.

During acute phase (0-4 weeks post infection) of SIV infection, blocking IFN-I signaling with an antagonistic mutant recombinant IFNα2/IFN-ant, with increased IFNAR2 binding but diminished IFNAR1 binding activity (Levin et al., Science Signaling 7:ra50 (2014)), leads to elevated SIV replication and accelerated disease progression in rhesus monkeys (Sandler et al., Nature 511:601 (2014)). In the same study, they report that, while administration of PEG-IFN-α2a during pre-infection and acute infection results in decreased SIV transmission, continued IFN-α2a treatment appears to induce IFN-I desensitization and to decrease antiviral gene expression, also resulting in increased SIV replication and accelerated CD4 T-cell loss (Sandler et al., Nature 511:601 (2014)). This study has major differences from the present study in that IFN-I signaling is blocked only during acute SIV infection. In the absence of IFN-I blocking during late stage of infection, the higher levels of SIV infection (probably also with higher IFN-I activity) may lead to the accelerated disease progression. It is generally believed that chronic IFN-signaling during persistent infection can lead to general immune suppression (Crouse et al., Nature Rev. Immunol. 15:231 (2015)). Thus, IFN-I signaling is beneficial during acute phase to inhibit HIV-1 infection and to prime host immune responses, but becomes harmful during chronic phase of infection. Blocking IFN-I signaling with either the INFAR1 or IFNAR2 blocking mAb in rhesus monkeys during chronic SIV infection will be of great interest to further clarify these findings.

Interestingly, a TLR7 and TLR9 antagonist in SIV-infected monkeys inhibits IFN-I production by pDCs but does not affect viral load, immune activation and CD4 T cell loss (Kader et al., PLoS Pathogens 9:e1003530 (2013)). It concludes that cells other than pDCs, including myeloid dendritic cells (mDCs), may also produce IFN-I during persistent SIV infection. The TLR7 and TLR9 antagonist fails to block IFN-I production by mDCs (Kader et al., PLoS Pathogens 9:e1003530 (2013)). Thus, IFN-I induced by SIV infection in pDC and other cell types may contribute to the pathogenesis of SIV infection.

In summary, it is reported here that IFN-I functions as a double-edged sword during persistent HIV-1 infection. It is beneficial by inhibiting HIV-1 replication but detrimental by inducing T-cell depletion and dysfunction. In HIV-1 infected patients, despite efficient suppression of HIV-1 replication with combined antiretroviral therapy, low elevated levels of IFN-I and ISGs still persist in some individuals (Fernandez et al., J. Infect. Dis. 204:1927 (2011); Dunham et al., J. Acquir. Immune Defic. Syndr. 65:133 (2014)), which may impede immune recovery and foster viral persistence (Bosinger et al., Cur. HIV/AIDS Rep. 12:41 (2015); Deeks, Annu. Rev. Med. 62:141 (2011)). Arecent study indicates that blocking type I interferon signaling during antiretroviral therapy enhances T cell recovery and reduces HIV-1 reservoirs (Cheng et al., J. Clin. Invest. 127:269 (2017)). It is conceivable that blocking IFNAR may provide a novel approach to facilitate recovery of functional anti-HIV-1 immune responses, thereby enabling control of HIV-1 reservoirs to achieve HIV-1 cure, as well as to treat those immune non-responder patients with elevated IFN-I signaling despite effective anti-retrovirus therapy.

### EXAMPLE 14

### Blockade of IFNAR Rescues Anti-HIV-1 CD8 T Cell Functions to Reduce HIV-1 Reservoir

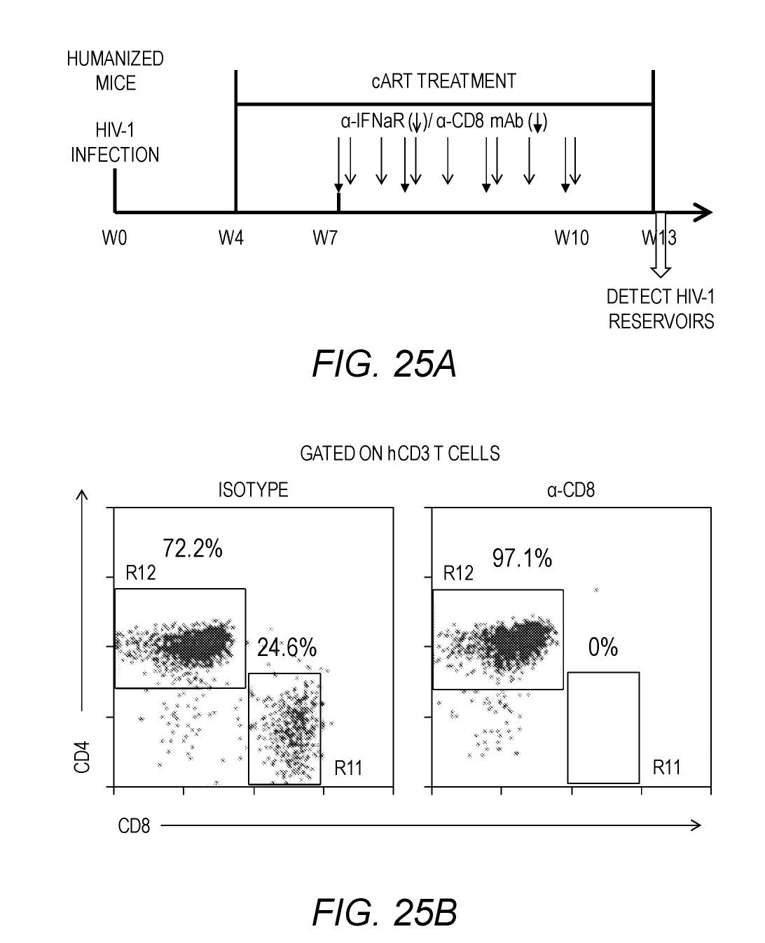

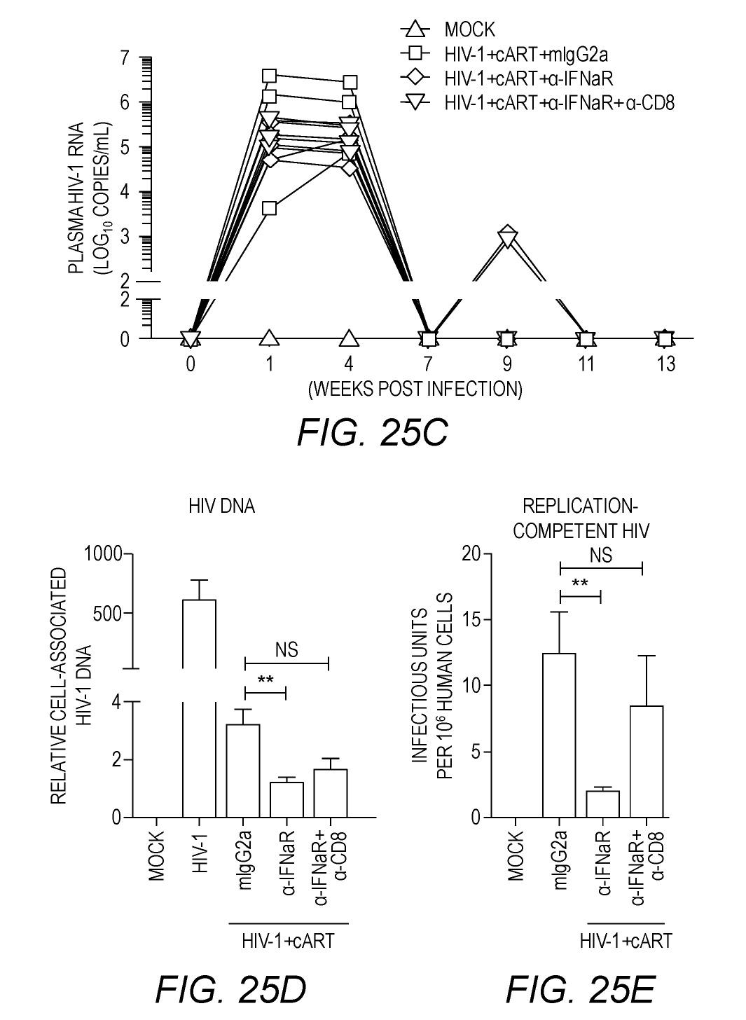

The importance of CD8 T cells in IFNAR bAb-mediated HIV reservoir reduction is established here. When CD8 T cells were depleted, the effect of IFNAR bAb in reducing HIV+cells was diminished (FIGS. 25A-25E).

Blocking IFNAR restores and enhances anti-HIV immunity. The HIV-1 reservoir cells was also measured in spleen. Intriguingly, both cell-associated HIV DNA and cells with infectious HIV were reduced in the IFNAR blocking group two weeks after mAb administration. More importantly, when cART was stopped, a significant delay of HIV-1 rebound was observed. Therefore, IFNAR blockade provides a new HIV cure strategy to eliminate HIV-1+reservoir cells.

The foregoing is illustrative of the present invention, and is not to be construed as limiting thereof. The invention is defined by the following claims, with equivalents of the claims to be included therein.

TABLE 1
IFNaR blockade in the presence of cART delays HIV-1 rebound post-cART cessation.

| | Mouse | % of cell (pre-infection) | | | | HIV-1 RNA in plasma (log10)^{ø} | | | | | | |
|---|---|---|---|---|---|---|---|---|---|---|---|---|
| Donor | number | CD45^{#} | CD3^{$} | CD4* | Treatment | W0 | W4 | W6 | W9 | W10 | W11 | W12 |
| 1 | 1962 | 81.3 | 59.0 | 79.7 | mIgG2a | 5.0 | N.D. | N.D. | N.D. | N.D. | N.D. | 6.2 |
| 1 | 1964 | 74.7 | 68.9 | 82.1 | mIgG2a | 5.1 | N.D. | N.D. | N.D. | N.D. | 5.4 | 6.6 |
| 1 | 1972 | 93.9 | 59.2 | 72.9 | mIgG2a | 4.7 | N.D. | N.D. | N.D. | 5.1 | 6.5 | 6.3 |
| 1 | 1973 | 95.5 | 54.0 | 73.5 | mIgG2a | 5.1 | N.D. | N.D. | N.D. | 3.6 | 5.8 | 5.6 |
| 1 | 1974 | 90.8 | 48.0 | 79.7 | mIgG2a | 4.6 | N.D. | N.D. | N.D. | 4.5 | 6.2 | 6.2 |
| 1 | 1975 | 74.9 | 54.2 | 72.2 | mIgG2a | 4.9 | N.D. | N.D. | N.D. | 4.9 | 5.4 | 6.7 |
| 1 | 1965 | 80.6 | 64.6 | 86.3 | α-IFNaR1 | 5.2 | N.D. | 3.1 | N.D. | N.D. | N.D. | 5.9 |
| 1 | 1966 | 96.8 | 66.4 | 80.5 | α-IFNaR1 | 5.0 | N.D. | 3.4 | N.D. | N.D. | 7.1 | 7.2 |
| 1 | 1967 | 62.8 | 47.3 | 70.9 | α-IFNaR1 | 5.1 | N.D. | 0.0 | N.D. | N.D. | N.D. | 6.1 |
| 2 | 1976 | 83.2 | 38.4 | 51.6 | mIgG2a | 5.5 | N.D. | N.D. | N.D. | N.D. | 5.1 | 5.1 |
| 2 | 1978 | 88.1 | 39.6 | 55.2 | mIgG2a | 5.0 | N.D. | N.D. | N.D. | 3.2 | 5.5 | 5.7 |
| 2 | 1979 | 85.7 | 33.4 | 58.3 | mIgG2a | 5.0 | N.D. | N.D. | N.D. | 3.7 | 5.5 | 5.3 |
| 2 | 1980 | 86.4 | 33.6 | 58.3 | mIgG2a | 5.6 | N.D. | N.D. | N.D. | 2.9 | 5.7 | 5.6 |
| 2 | 1981 | 80.3 | 35.0 | 32.2 | α-IFNaR1 | 5.4 | N.D. | 3.4 | N.D. | N.D. | N.D. | 5.1 |
| 2 | 1982 | 83.4 | 43.5 | 41.3 | α-IFNaR1 | 5.5 | N.D. | N.D. | N.D. | N.D. | N.D. | N.D. |
| 2 | 1984 | 69.4 | 32.2 | 39.4 | α-IFNaR1 | 5.4 | N.D. | 3.0 | N.D. | N.D. | N.D. | N.D. |
| 2 | 1985 | 77.1 | 29.8 | 57 | α-IFNaR1 | 6.0 | N.D. | N.D. | N.D. | N.D. | N.D. | 5.3 |
| 3 | 2112 | 15.4 | 54.4 | 74.0 | mIgG2a | 4.8 | N.D. | N.D. | N.D. | N.D. | N.D. | 5.2 |
| 3 | 2113 | 20.9 | 45.6 | 75.6 | mIgG2a | 4.6 | N.D. | 2.7 | N.D. | 3.6 | 5.3 | 6.1 |
| 3 | 2114 | 21.6 | 43.8 | 80.0 | mIgG2a | 5.9 | N.D. | N.D. | N.D. | N.D. | 4.1 | 7.0 |
| 3 | 2115 | 10.0 | 59.6 | 63.6 | mIgG2a | 5.5 | N.D. | N.D. | N.D. | N.D. | 5.6 | 5.8 |
| 3 | 2176 | 38.1 | 20.6 | 56.9 | mIgG2a | 4.4 | N.D. | N.D. | N.D. | 4.0 | 4.3 | 4.9 |
| 3 | 2177 | 46.3 | 24.0 | 64.6 | mIgG2a | 4.4 | N.D. | N.D. | N.D. | 4.3 | 4.4 | 5.2 |
| 3 | 2178 | 44.5 | 26.1 | 60.5 | mIgG2a | 4.7 | N.D. | N.D. | N.D. | N.D. | 4.3 | 5.6 |
| 3 | 2179 | 37.5 | 25.9 | 62.4 | mIgG2a | 4.5 | N.D. | N.D. | N.D. | N.D. | 2.8 | 5.6 |
| 3 | 2124 | 22.5 | 27.5 | 70.1 | α-IFNaR1 | 6.3 | N.D. | N.D. | N.D. | N.D. | N.D. | 4.7 |
| 3 | 2125 | 4.4 | 45.9 | 62.8 | α-IFNaR1 | 4.8 | N.D. | N.D. | N.D. | N.D. | N.D. | N.D. |
| 3 | 2128 | 15.1 | 37.9 | 68.9 | α-IFNaR1 | 5.8 | N.D. | N.D. | N.D. | N.D. | 5.5 | 4.7 |
| 3 | 2174 | 60.9 | 27.9 | 73.7 | α-IFNaR1 | 5.9 | N.D. | N.D. | N.D. | N.D. | 4.5 | 5.9 |

Humanized mice were treated as in FIG. 6A.
^{#}Percentage of human CD45⁺ of total cells in PBMCs.
^{$}Percentage of CD3⁺ from human CD45^{+ cells.}
*Percentage of CD4 from CD3⁺ cells.
^{ø}weeks post cART treatment.
N.D., No Detectable

1. A method of reactivating latent HIV-1 or reducing HIV-1 reservoirs in a subject in need thereof, comprising inhibiting type I interferon signaling in the subject, thereby reactivating latent HIV-1 in the subject.

2. (canceled)

3. A method of treating HIV-1 infection or increasing the effectiveness of combination antiretroviral therapy (cART) for HIV-1 infection in a subject in need thereof, comprising inhibiting type I interferon signaling in the subject, thereby treating HIV-1 infection in the subject.

4. (canceled)

5. (canceled)

6. The method of claim 1, wherein the subject is a non-responder to cART.

7. The method of claim 1, wherein the subject previously underwent cART.

8. The method of claim 1, wherein inhibiting interferon-I signaling comprises delivering to the subject an effective amount of an antibody or a fragment thereof that specifically binds to the interferon-α/β receptor.

9. The method of claim 1, wherein type I interferon signaling is inhibited by at least 50%.

10. The method of claim 1, wherein type I interferon signaling is inhibited by at least 90%.

11. The method of claim 1, further comprising delivering to the subject a HIV-1 therapeutic agent.

12. The method of claim 11, wherein the HIV-1 therapeutic agent is an antiretroviral agent.

13. The method of claim 12, wherein the antiretroviral agent is selected from the group consisting of reverse transcriptase inhibitors, protease inhibitors, viral integration inbibitors, viral entry inhibitors, viral maturation inhibitors, iRNA agents, antisense RNA, vectors expressing iRNA agents or antisense RNA, PNA, antiviral antibodies and any combination thereof.

14. The method of claim 12, wherein the antiretroviral agent is selected from the group consisting of AZT, 3TC, ddI, ddC, 3TC, saquinavir, indinavir, ritonavir, nelfinavir, nevirapine, efavirenz, and combinations thereof.

15. The method of claim 11, wherein the HIV-1 therapeutic agent is an antibody or a fragment thereof that specifically binds to BDCA2 and depletes plasmacytoid dendritic cells.

16. The method of claim 1, wherein the subject is a human.

17. The method of claim 1, wherein the subject is an animal model of HIV-1 infection.

18. A method for identifying a compound suitable for treatment of HIV-1 infection, the method comprising providing an animal model of HIV-1 infection in which type 1 interferon signaling has been inhibited, delivering a candidate compound to the animal, and measuring HIV-1 levels in the animal, wherein a decrease in HIV-1 levels compared to an animal that has not received the candidate compound identifies the candidate compound as a compound suitable for treatment of HIV-1 infection.

19. The method of claim 18, wherein inhibiting type I interferon signaling comprises delivering to the subject an effective amount of an antibody or a fragment thereof that specifically binds to the interferon-α/β receptor.

20. The method of claim 18, wherein type I interferon signaling is inhibited by at least 50%.

21. The method of claim 18, type 1 interferon signaling is inhibited by at least 90%.

22. The method of claim 18, wherein the animal is a mouse.

23. The method claim 18, wherein the animal model is a humanized mouse model.
